(19) United States
(12) Patent Application Publication
(10) Pub. No.: US 2022/0265337 A1
(43) Pub. Date: Aug. 25, 2022

(54) SYSTEMS, DEVICES, AND METHODS FOR TREATING METABOLIC MEDICAL CONDITIONS

(71) Applicant: Fractyl Health, Inc., Lexington, MA (US)

(72) Inventors: HARITH RAJAGOPALAN, Wellesley Hills, MA (US); DAVID MAGGS, Boston, MA (US); J. CHRISTOPHER FLAHERTY, Auburndale, FL (US)

(73) Assignee: Fractyl Health, Inc., Lexington, MA (US)

(21) Appl. No.: 17/490,947

(22) Filed: Sep. 30, 2021

Related U.S. Application Data
(63) Continuation of application No. PCT/US20/25925, filed on Mar. 31, 2020.
(60) Provisional application No. 62/827,355, filed on Apr. 1, 2019.
(60) Provisional application No. 62/848,793, filed on May 16, 2019.

(51) Int. Cl. A61B 18/06; A61B 18/20

(52) U.S. Cl. CPC A61B 18/06; A61B 18/20; A61B 2018/00494

(57) ABSTRACT

Provided herein are systems for treating a patient, the systems comprising: a treatment device for treating a minimum amount of mucosal tissue of the patient's small intestine. The system is configured to treat a medical condition of the patient. Methods of treating a patient are also provided.

### RELATED APPLICATIONS

This application is a continuation of PCT Application No. PCT/US20/25925 (Attorney Docket No. 41714-719601), filed Mar. 31, 2020, which claims the benefit of: U.S. Provisional No. 62/827,355 (Attorney Docket No. 41714-719.101; Client Docket No. MCT-040-PR1), filed Apr. 1, 2019; U.S. Provisional No. 62/848,793 (Attorney Docket No. 41714-719.102; Client Docket No. MCT-040-PR2), filed May 16, 2019; the entire content of which are each incorporated herein by reference in their entirety.

This application is related to: U.S. patent application Ser. No. 13/945,138 (Attorney Docket No. 41714-703.301; Client Docket No. MCT-001-US), entitled “Devices and Methods for the Treatment of Tissue”, filed Jul. 18, 2013; U.S. patent application Ser. No. 14/470,503 (Attorney Docket No. 41714-704.301; Client Docket No. MCT-002-US), entitled “Heat Ablation Systems, Devices and Methods for the Treatment of Tissue”, filed Aug. 27, 2014; U.S. patent application Ser. No. 14/515,324 (Attorney Docket No. 41714-705.301; Client Docket No. MCT-003-US), entitled “Tissue Expansion Devices, Systems and Methods”, filed Oct. 15, 2014; U.S. patent application Ser. No. 14/609,334 (Attorney Docket No. 41714-707.301; Client Docket No. MCT-005-US), entitled “Ablation Systems, Devices and Methods for the Treatment of Tissue”, filed Jan. 29, 2015; U.S. patent application Ser. No. 14/673,565 (Attorney Docket No. 41714-708.301; Client Docket No. MCT-009-US), entitled “Methods, Systems and Devices for Performing Multiple Treatments on a Patient”, filed Mar. 30, 2015; U.S. patent application Ser. No. 14/956,710 (Attorney Docket No. 41714-709.301; Client Docket No. MCT-013-US), entitled “Methods, Systems and Devices for Reducing the Luminal Surface Area of the Gastrointestinal Tract”, filed Dec. 2, 2015; U.S. patent application Ser. No. 14/917,243 (Attorney Docket No. 41714-710.301; Client Docket No. MCT-023-US), entitled “Systems, Methods and Devices for Treatment of Target Tissue”, filed Mar. 7, 2016; U.S. patent application Ser. No. 15/156,585 (Attorney Docket No. 41714-711.301; Client Docket No. MCT-024-US), entitled “Systems, Devices and Methods for the Creation of a Therapeutic Restriction in the Gastrointestinal Tract”, filed May 17, 2016; U.S. patent application Ser. No. 15/274,948 (Attorney Docket No. 41714-712.301; Client Docket No. MCT-027-US), entitled “Injectate Delivery Devices, Systems and Methods”, filed Sep. 23, 2016; U.S. patent application Ser. No. 15/274,764 (Attorney Docket No. 41714-714.501; Client Docket No. MCT-028-US-CIP1), entitled “Systems, Devices and Methods for Performing Medical Procedures in the Intestine”, filed Sep. 23, 2016; U.S. patent application Ser. No. 15/274,809 (Attorney Docket No. 41714-714.502; Client Docket No. MCT-028-US-CIP2), entitled “Systems, Devices and Methods for Performing Medical Procedures in the Intestine”, filed Sep. 23, 2016; U.S. patent application Ser. No. 15/406,572 (Attorney Docket No. 41714-713.301; Client Docket No. MCT-029-US), entitled “Methods and Systems for Treating Diabetes and Related Diseases and Disorders”, filed Jan. 13, 2017; U.S. patent application Ser. No. 15/683,713 (Attorney Docket No. 41714-714.301; Client Docket No. MCT-028-US-CIP1-CON1), entitled “Systems, Devices, and Methods for Performing Medical Procedures in the Intestine”, filed Aug. 22, 2017; U.S. patent application Ser. No. 15/812,969 (Attorney Docket No. 41714-714.302; Client Docket No. MCT-028-US-CIP2-CON1), entitled “Systems, Devices, and Methods for Performing Medical Procedures in the Intestine”, filed Nov. 14, 2017; U.S. patent application Ser. No. 15/917,480 (Attorney Docket No. 41714-703.302; Client Docket No. MCT-001-US-CON1), entitled “Devices and Methods for the Treatment of Tissue”, filed Mar. 9, 2018; International PCT Patent Application Serial Number PCT/US2019/012338 (Attorney Docket No. 41714-717.601; Client Docket No. MCT-036-PCT), entitled “Material Depositing System for Treating a Patient”, filed Jan. 4, 2019; U.S. patent application Ser. No. 16/267,771 (Attorney Docket No. 41714-711.302; Client Docket No. MCT-024-US-CON1) entitled “Systems, Devices and Methods for the Creation of a Therapeutic Restriction in the Gastrointestinal Tract”, filed Feb. 5, 2019; U.S. patent application Ser. No. 14/956,710 (Attorney Docket No. 41714-709.302; Client Docket No. MCT-013-US-CON1), entitled “Methods, Systems and Devices for Reducing the Luminal Surface Area of the Gastrointestinal Tract”, filed Apr. 9, 2019; U.S. patent application Ser. No. 16/400,491 (Attorney Docket No. 41714-716.301; Client Docket No. MCT-035-US), entitled “Systems, Device, and Methods for Performing Medical Procedures in the Intestine”, filed May 1, 2019; U.S. patent application Ser. No. 16/438,362 (Attorney Docket No. 41714-704.302; Client Docket No. MCT-002-US-CON1), entitled “Heat Ablation Systems, Devices and Methods for the Treatment of Tissue”, filed Jun. 11, 2019; International PCT Patent Application Serial Number PCT/US2019/54088 (Attorney Docket No. 41714-718.601; Client Docket No. MCT-037-PCT), entitled “System and Methods for Depositing Material in a Patient”, filed Oct. 1, 2019; U.S. Provisional Patent Application Ser. No. 62/923,710 (Attorney Docket No. 41714-720.101; Client Docket No. MCT-050-PR1), entitled “Systems, Devices, and Methods for Performing Medical Procedures in the Intestine”, filed Oct. 21, 2019; U.S. patent application Ser. No. 16/711,236 (Attorney Docket No. 41714-706.302; Client Docket No. MCT-004-US-CON1), entitled “Electrical Energy Ablation Systems, Devices and Methods for the Treatment of Tissue”, filed Dec. 11, 2019; U.S. Provisional Patent Application Ser. No. 62/960,340 (Attorney Docket No. 41714-721.101; Client Docket No. MCT-039-PR1), entitled “Tissue Treatment Devices, Systems, and Methods”, filed Jan. 13, 2020; U.S. patent application Ser. No. 16/742,645 (Attorney Docket No. 41714-715.201; Client Docket No. MCT-025-US), entitled “Intestinal Catheter Device and System”, filed Jan. 14, 2020; U.S. Provisional Patent Application Ser. No. 62/961,340 (Attorney Docket No. 41714-722.101; Client Docket No. MCT-051-PR1), entitled “Automated Tissue Treatment Devices, Systems, and Methods”, filed Jan. 15, 2020; U.S. patent application Ser. No. 16/798,117 (Attorney Docket No. 41714-714.303; Client Docket No. MCT-028-US-CIP1-CON2), entitled “Systems, Devices, and Methods for Performing Medical Procedures in the Intestine”, filed Feb. 21, 2020; and U.S. Provisional Patent Application Ser. No. 62/991,219 (Attorney Docket No. 41714-723.101; Client Docket No. MCT-041-PR1), entitled “Systems, Devices and Methods for Treating Diabetes”, filed Mar. 18, 2020; the contents of each of which is incorporated herein by reference in its entirety for all purposes.

### TECHNICAL FIELD

The embodiments disclosed herein relate generally to systems, devices and methods for treating metabolic conditions of a patient.

### BACKGROUND

Numerous diagnostic and therapeutic procedures are performed in the small and large intestine, as well as other locations of the gastrointestinal tract. Devices used in these procedures can be difficult to maneuver and otherwise operate, and they often have limited functionality. There is a need for improved systems and devices for treating and diagnosing tissue of the intestine, as well as a need for methods of treating intestinal tissue to provide an improved therapy for various diseases and disorders.

### BRIEF SUMMARY

According to an aspect of the present inventive concepts, a system for treating a patient is provided. The system comprises a treatment device for treating a minimum amount of mucosal tissue of the patient's small intestine. The system is configured to treat a medical condition of the patient.

In some embodiments, the mucosal tissue of the small intestine treated comprises duodenal mucosal tissue.

In some embodiments, the medical condition treated comprises NAFLD/NASH and/or Type 2 diabetes. The medical condition treated can comprise NAFLD/NASH and Type 2 diabetes. The system can be configured to lower hepatic fat of the patient. The system can be configured to lower hepatic fat of the patient by at least 10%. The system can be configured to lower hepatic fat of the patient by at least 20%. The system can be configured to lower hepatic fat of the patient by at least 30%. The system can be configured to lower hepatic fat of the patient by approximately 36%. The system can be configured to lower HbA1c of the patient. The system can be configured to lower HbA1c of the patient by at least 0.5%. The system can be configured to lower HbA1c of the patient by at least 0.7%. The system can be configured to lower HbA1c of the patient by approximately 1%. The system can be configured to lower hepatic fat of the patient and lower HbA1c of the patient. The system can be configured to lower the triglyceride/HDL ratio of the patient. The system can be configured to lower the triglyceride/HDL ratio of the patient by at least 10%. The system can be configured to lower the triglyceride/HDL ratio of the patient by at least 20%. The system can be configured to lower the triglyceride/HDL ratio of the patient by approximately 28%. The system can be configured to cause weight loss for the patient. The system can be configured to cause a weight loss for the patient of at least 2%, 3%, and/or 5% of the patient's pre-procedure body weight. The system can be configured to cause weight loss for the patient of approximately 3.1 kg. The system can be configured to cause weight loss for the patient without a lifestyle intervention. The system can be configured to provide a therapeutic benefit for the patient that can be present at a time within three months of treating the mucosal tissue of the patient's small intestine. The system can be configured to provide a therapeutic benefit for the patient that can be present at a time at least six months from treating the mucosal tissue of the patient's small intestine. The system can be configured to lower HbA1c of the patient by at least 0.5% and to reduce liver fat of the patient by at least 20%. The system can be configured to lower HbA1c by at least 0.7% and to reduce liver fat by at least 30%.

In some embodiments, the system is configured to treat at least 30% of the post-papillary duodenum.

In some embodiments, the system is configured to treat a cumulative axial length of duodenal mucosal tissue of at least 3 cm, at least 6 cm, at least 8 cm, and/or at least 9 cm.

In some embodiments, the treatment device includes at least one treatment assembly. The treatment assembly can comprise a balloon. The treatment assembly can comprise an array of energy delivery elements. The treatment assembly can comprise at least one energy delivery element configured to deliver an energy form selected from the group consisting of: electromagnetic energy; RF energy; light energy; laser light energy; sound energy; ultrasound energy; chemical energy; and combinations thereof. The treatment assembly can comprise a treatment length of at least 10 mm. The treatment assembly can comprise a treatment length of no more than 100 mm. The treatment assembly can comprise an expanded diameter of at least 20 mm. The treatment assembly can comprise an expanded diameter of no more than 40 mm, or no more than 30 mm.

In some embodiments, the system is configured to perform multiple tissue treatments.

In some embodiments, the treatment device is configured to perform a thermal ablation of the mucosal tissue.

In some embodiments, the treatment device is configured to perform an electromagnetic energy ablation of the mucosal tissue.

In some embodiments, the treatment device is configured to perform a chemical ablation of the mucosal tissue.

In some embodiments, the system is configured to cause a physiologic response in the patient selected from the group consisting of: a reduction in the surface area of the mucosal tissue proximate treated locations; an altering of hormonal signaling of the intestine proximate treated locations; replacement of existing mucosal tissue with new mucosal tissue proximate treated locations; a reduction in iron absorption; a reduction in bile acid signaling; an increase in bile acid signaling; an altering of microbiome composition of the intestine; a reduction in glucose, fat, and/or amino acid signaling; a reduction in glucose, fat, and/or amino acid absorption; a reduction in GIP levels in the fasting state, such as by at least 5%, 10%, and/or 20%; a reduction in GIP levels in the post-prandial state, such as by at least 5%, 10%, and/or 20%; an increase in GLP-1 levels in the post-prandial state, such as by at least 5%, 10%, and/or 15%; an increase in GLP-1 levels in the post-prandial state without significantly altering GLP-1 levels in the fasting state; and combinations thereof.

In some embodiments, the system further comprises a tissue expansion assembly. The treatment device can comprise the tissue expansion assembly.

According to another aspect of the present inventive concepts, a method of treating a patient with Type 2 diabetes is provided. The method comprises selecting a patient afflicted with Type 2 diabetes and currently taking insulin, treating duodenal mucosa of the patient, delivering an agent to the patient, and stopping the taking of insulin. The method is configured to provide a therapeutic benefit to the patient.

In some embodiments, the patient has an HbA1c level of no more than 64 mmol/mol prior to the treating of the duodenal mucosa.

In some embodiments, the patient has a C-peptide level of at least 0.5 nmol/l prior to the treating of the duodenal mucosa.

In some embodiments, the insulin taken by the patient prior to the treating of the duodenal mucosa is taken at a level of no more than 1 U/kg/day.

In some embodiments, the agent comprises a GLP-1 receptor agonist. The GLP-1 receptor agonist can comprise liraglutide. The liraglutide can be delivered to the patient at a rate of at least 0.6 mg/day. The liraglutide can be delivered to the patient at a rate of no more than 3.0 mg/day. The liraglutide can be delivered to the patient at a rate of approximately 1.8 mg/day.

In some embodiments, the treating duodenal mucosa comprises a heat ablation of the duodenal mucosa.

In some embodiments, the method further comprises performing a tissue expansion of submucosal tissue proximate the duodenal mucosa to be treated, and subsequently treating the duodenal mucosa. The treating the duodenal mucosa can comprise ablating the duodenal mucosa.

In some embodiments, the treating duodenal mucosa comprises treating one or more axial segments of the duodenum. The treating of one or more axial segments can comprise delivering an ablation energy to a full circumferential portion of at least one of the one or more axial segments. The delivery of ablation energy to a full circumferential portion can result in less than 100% ablation efficiency. The treating of one or more axial segments can comprise delivering an ablation energy to a partial circumferential portion of at least one of the one or more axial segments. The one or more axial segments of the duodenum treated can comprise at least two, three, four, and/or five axial segments of the duodenum. The one or more axial segments of the duodenum treated can comprise a cumulative axial length of at least 2 cm. The one or more axial segments of the duodenum treated can comprise a cumulative axial length of at least 3 cm. The one or more axial segments of the duodenum treated can comprise a cumulative axial length of at least 6 cm. The one or more axial segments of the duodenum treated can comprise a cumulative axial length of at least 9 cm.

In some embodiments, the treating duodenal mucosa comprises treating at least 6.28 cm² of surface area of duodenal mucosa. The treating duodenal mucosa can comprise treating at least 9.42 cm² of surface area of duodenal mucosa. The treating duodenal mucosa can comprise treating at least 18.84 cm² of surface area of duodenal mucosa. The treating duodenal mucosa can comprise treating at least 28.27 cm² of surface area of duodenal mucosa.

In some embodiments, the treating duodenal mucosa comprises treating at least 25% of the post-papillary duodenal mucosa. The treating duodenal mucosa can comprise treating at least 50% of the post-papillary duodenal mucosa. None of the pre-papillary duodenum can be treated.

In some embodiments, the therapeutic benefit comprises maintaining and/or improving one or more metabolic conditions of the patient after the taking of insulin is stopped. The therapeutic benefit can comprise maintaining one or more metabolic conditions of the patient. The one or more metabolic conditions maintained can comprise one or more metabolic conditions selected from the group consisting of: HbA1c level; BMI; FPG level; insulin level; HOMA-IR level; ALT level; and combinations thereof. The therapeutic benefit can comprise maintaining two more metabolic conditions of the patient. The two or more metabolic conditions maintained can comprise two or more conditions selected from the group consisting of: HbA1c level; BMI; FPG level; insulin level; HOMA-IR level; ALT level; and combinations thereof. The therapeutic benefit can comprise improving one or more metabolic conditions of the patient. The one or more metabolic conditions improved can comprise one or more metabolic conditions selected from the group consisting of: HbA1c level; BMI; FPG level; insulin level; HOMA-IR level; ALT level; and combinations thereof. The therapeutic benefit can comprise improving two more metabolic conditions of the patient. The two or more metabolic conditions improved can comprise two or more conditions selected from the group consisting of: HbA1c level; BMI; FPG level; insulin level; HOMA-IR level; ALT level; and combinations thereof. The metabolic condition maintained and/or improved can comprise a BMI of the patient. The metabolic condition maintained and/or improved can comprise an HbA1c level of the patient. The therapeutic benefit can comprise a reduction in the HbA1c level of the patient. The metabolic condition maintained and/or improved can comprise an FPG level of the patient. The metabolic condition maintained and/or improved can comprise an insulin level of the patient. The metabolic condition maintained and/or improved can comprise a HOMA-IR level of the patient. The metabolic condition maintained and/or improved can comprise an ALT level of the patient.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a schematic view of a system for performing a medical procedure in the intestine of a patient, consistent with the present inventive concepts.

FIG. 2 illustrates a schematic view of a system and device for performing a medical procedure on the small intestine of a patient, consistent with the present inventive concepts.

FIGS. 3 and 3A-3B illustrate an anatomic view of a system for performing a medical procedure comprising a catheter and a sheath for inserting the catheter into the intestine of the patient, consistent with the present inventive concepts.

FIG. 4 illustrates a sectional view of the distal portion of a system including an endoscope and a treatment device inserted into a duodenum of a patient, consistent with the present inventive concepts.

FIGS. 5A-5B illustrate end and side views of the distal portion of a catheter including recessed ports and shaft-located vacuum port, consistent with the present inventive concepts.

FIG. 6 illustrates a flow chart of a method of treating a patient, consistent with the present inventive concepts.

FIG. 7 illustrates a flow chart of a method of preparing a treatment device, consistent with the present inventive concepts.

FIG. 8 illustrates a flow chart of a method of expanding tissue with a treatment device, consistent with the present inventive concepts.

FIG. 9 illustrates a flow chart of a method of ablating or otherwise treating tissue with a treatment device, consistent with the present inventive concepts.

FIG. 10 illustrates a graph showing a reduction of AST and ALT levels in patients receiving a duodenal mucosal treatment, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of two or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “transducer” is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: heat energy to tissue; cryogenic energy to tissue; electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of two or more of these. A transducer can include a component configured to neutralize an ablative process, such as a transducer configured to cool tissue prior to and/or after a heat ablation of tissue, and/or a transducer configured to warm tissue prior to and/or after a cryogenic ablation of tissue. Alternatively or additionally, a transducer can comprise a mechanism, such as: a valve; a grasping element; an anchoring mechanism; an electrically-activated mechanism; a mechanically-activated mechanism; and/or a thermally activated mechanism.

As used herein, the term “functional element” is to be taken to include one or more elements constructed and arranged to perform a function. A functional element can comprise one or more sensors and/or one or more transducers. In some embodiments, a functional element is configured to deliver energy and/or otherwise treat tissue (e.g. a functional element configured as a treatment element). Alternatively or additionally, a functional element (e.g. comprising one or more sensors) can be configured to record one or more parameters, such as a patient physiologic parameter; a patient anatomical parameter (e.g. a tissue parameter); a patient environment parameter; and/or a system parameter (e.g. temperature and/or pressure within the system). In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a patient anatomical parameter; and combinations of two or more of these. A functional element can comprise a fluid, such as an ablative fluid (as described herein) comprising a liquid, gel, and/or gas configured to ablate or otherwise treat tissue. A functional element can comprise a reservoir, such as an expandable balloon configured to receive an ablative fluid. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly. A functional assembly can comprise one or more functional elements.

As used herein, the term “ablative temperature” refers to a temperature at which tissue necrosis or other desired tissue treatment occurs (e.g. a temperature sufficiently hot or sufficiently cold to cause tissue necrosis). As used herein, the term “ablative fluid” refers to one or more liquids, gases, gels or other fluids whose thermal properties cause tissue necrosis and/or another desired tissue treatment (e.g. one or more fluids at an ablative temperature). Alternatively or additionally, “ablative fluid” refers to one or more fluids whose chemical properties (at room temperature, body temperature or otherwise) cause tissue necrosis or another desired tissue treatment. A tissue treatment element (e.g. a functional element) of the present inventive concepts can comprise one or more ablative fluids.

As used herein, the term “tissue contacting surface” refers to a surface of a system or device component that makes physical contact with tissue, such as a portion of an external surface of an expandable component (e.g. a portion of a balloon's surface) which contacts tissue once expanded. In some embodiments, tissue contacting a tissue contacting surface directly receives energy from the tissue contacting surface of the expandable components, however tissue in proximity (e.g. below or alongside) also receives energy (e.g. via conduction of the delivered energy and/or a resultant heat energy).

It is an object of the present inventive concepts to provide systems, methods and devices for safely and effectively treating and/or diagnosing a volume of tissue (the “target tissue”), such as to treat and/or diagnose a patient disease or disorder. Target tissue can comprise one or more target tissue segments or other target tissue portions, such as target tissue located in the intestine of a patient. Clinical procedures in the duodenum and other locations of the small intestine are challenging for a number of reasons, such as those caused by the long distance between the mouth and the intestine and the complexities of the gastrointestinal passageway encountered (including passage through the stomach) during device (e.g. catheter) insertion and operation. Intestinal diameter varies along its length, and effective devices must accommodate this variation. The intestine is quite distensible in the longitudinal and radial directions, further complicating device (e.g. catheter) manipulation and operation (e.g. delivery of energy to tissue). Mobility of intestinal mucosa relative to muscularis is present, as well as mobility of the full wall, but can result in undesired stretching, compression and intussusception. The duodenum is normally closed, and it can require insufflation to open (e.g. for visualization). The insufflation medium (e.g. gas) moves through the intestine, so more must be delivered, while excess gas causes discomfort or other adverse effect for the patient. Duodenal and other intestinal tissue tends to stretch or compress as a device is advanced or retracted, respectively, such as to cause retrograde expulsion of devices if a stabilization force is not maintained. It is difficult to manipulate and control devices that include treatment and other elements positioned in the small intestine. The small intestine wraps around the pancreas, and the curvature is quite variable from patient to patient. The length of the intestine along an outer curve is longer than that along an inner curve. In many procedures, there is a desire to avoid damage to the ampulla of Vater (e.g. to avoid restricting bile and/or pancreatic fluid), tissue which can be difficult to visualize or otherwise identify. There are relatively few endoscopically visualizable landmarks in the intestine, making it difficult to know where in the intestine a portion (e.g. a distal portion) of a device is positioned. Access to the intestine through the stomach via an over-the wire catheter loses one-to-one motion between a proximal handle and a distal portion of the device, as slack can accumulate in the stomach during advancement and slack can be relieved from the stomach during withdrawal. Accessing the intestine can include entering the intestine through the pylorus, a small sphincter, from the stomach, and in obese patients, large stretchable stomachs make it difficult to direct a device to the pylorus. The intestinal mucosa has a very irregular surface due to plicae circulares and mucosal villi, and performing a treatment (e.g. an ablation treatment) of the intestinal mucosa is quite different from a treatment procedure performed in the stomach or esophagus, because of this irregularity. Peristalsis present in the small intestine is dynamic and unpredictable and can alter functional element, functional assembly and/or other device component position and/or contact level with tissue. The intestine is not only thin-walled, but the thickness of the wall is highly variable, even within small axial segments of the small intestine, thus complicating preferential ablation of inner layers versus outer layers of the small intestine. The muscularis is innervated and scars and/or stenoses easily, and as such, even minimal trauma to the muscularis should be avoided.

Target tissue can comprise one or more layers of a portion of tubular or non-tubular tissue, such as tissue of an organ or tissue of the gastrointestinal (GI) tract of a patient, such as tissue of the small intestine or large intestine. The systems and devices of the present inventive concepts can include one or more functional assemblies and/or functional elements configured to treat target tissue, such as a treatment element comprising fluid at an ablative temperature delivered to a balloon (ablative temperature fluid and/or balloon filled with ablative fluid each referred to singly or collectively as a “functional element” or a “treatment element” of the present inventive concepts). One or more functional elements can be provided in, on and/or within an expandable functional assembly or other radially deployable mechanism. Functional assemblies and/or functional elements can be configured to treat target tissue (e.g. deliver energy to target tissue), such as to modify target tissue (e.g. to modify neuronal signaling, to modify neurohormonal signaling, to modify the secretions from the target tissue and/or absorption of the target tissue), ablate target tissue (e.g. to cause the replacement of the target tissue with “new tissue”) and/or to cause a reduction in the surface area of target tissue (e.g. the luminal surface area of an inner wall of tubular tissue) at and/or proximate to one or more locations where the treatment was performed (e.g. at and/or proximate the location where energy was delivered). The luminal or other tissue treatment can occur acutely and/or it can take place over time, such as days, weeks or months. A tissue surface area reduction can correspond to a reduction in mucosal surface area available to function in an absorptive, neuronal signaling, and/or a hormonal secretory capacity. A target tissue treatment can result in the replacement of target tissue with new tissue with different absorptive and/or secretory capacity and/or other desirable effect related to replacement and/or modification of target tissue. The treatment of target tissue with the systems, devices and methods of the present inventive concepts can provide a therapeutic benefit to the patient, such as to treat one or more diseases or disorders of the patient, as described in detail herebelow.

Each functional assembly (e.g. treatment assembly) can comprise at least one functional element (e.g. tissue treatment element) such as a tissue treatment element selected from the group consisting of: ablative fluid delivered to a balloon or other expandable fluid reservoir; energy delivery element mounted to an expandable functional assembly such as an electrode or other energy delivery element configured to deliver radiofrequency (RF) energy and/or microwave energy; light delivery element configured to deliver laser or other light energy; fluid delivery element (e.g. needle or nozzle) configured to deliver ablative fluid directly onto and/or into tissue; sound delivery element such as an ultrasonic and/or subsonic sound delivery element; and combinations of two or more of these. Numerous forms of functional assemblies and/or functional elements can be included. In some embodiments, the functional assemblies and/or the one or more functional elements contained therein are configured as described in: applicant's co-pending U.S. patent application Ser. No. 15/917,480 (Attorney Docket No. 41714-703.302; Client Docket No. MCT-001-US-CON1), entitled “Devices and Methods for the Treatment of Tissue”, filed Mar. 9, 2018; applicant's co-pending U.S. patent application Ser. No. 16/438,362 (Attorney Docket No. 41714-704.302; Client Docket No. MCT-002-US-CON1), entitled “Heat Ablation Systems, Devices and Methods for the Treatment of Tissue”, filed Jun. 11, 2019; applicant's co-pending U.S. patent application Ser. No. 16/711,236 (Attorney Docket No. 41714-706.302; Client Docket No. MCT-004-US-CON1), entitled “Electrical Energy Ablation Systems, Devices and Methods for the Treatment of Tissue”, filed Dec. 11, 2019; and/or applicant's co-pending U.S. patent application Ser. No. 14/609,334 (Attorney Docket No. 41714-707.301; Client Docket No. MCT-005-US), entitled “Ablation Systems, Devices and Methods for the Treatment of Tissue”, filed Jan. 29, 2015; the content of each of which is incorporated herein by reference in its entirety for all purposes.

The treatment assemblies and/or treatment elements of the present inventive concepts can be constructed and arranged to deliver one or more treatments (e.g. deliver energy, deliver a chemically ablative fluid, mechanically abrade and/or otherwise treat tissue) directly to a particular area of tissue, the “delivery zone”. The area of tissue treated can comprise a segment of the small intestine, or other body lumen, where the delivery zone comprises a length representing the axial length of the segment treated, and a width such as a width representing a full or partial circumferential portion of the segment. A treatment element can be configured to ablate or otherwise treat an energy delivery zone with a “treatment length” and a “treatment width”. During a single delivery of treatment, a treatment element can be constructed and arranged to deliver treatment to a relatively continuous surface of tissue (e.g. a continuous surface of tissue in contact with a balloon filled with ablative fluid or a surface of tissue onto which a chemically ablative fluid is sprayed, coated or otherwise delivered). In these continuous-surface treatment delivery embodiments, the delivery zone comprises the continuous surface of tissue receiving the treatment directly. Alternatively, a treatment element can be constructed and arranged to deliver treatment to multiple discrete portions of a tissue surface, with one or more tissue surface portions in-between other surface portions that do not directly receive energy or other treatment from the treatment element. In these segmented-surface treatment delivery embodiments, the delivery zone is defined by a periphery of the multiple tissue surface area portions receiving treatment, similar to a “convex hull” or “convex envelope” used in mathematics to define an area including a number of discrete locations that define a periphery. A delivery zone can comprise two or more contiguous or non-contiguous delivery zones, and multiple delivery zones can be treated sequentially and/or simultaneously.

For example, in embodiments where the treatment element is hot fluid (e.g. ablative fluid at a sufficiently high temperature to cause tissue necrosis) positioned within a balloon, the delivery zone comprises all tissue surfaces contacted by the balloon that directly receive ablative thermal energy from the ablative fluid through the balloon. In embodiments where the treatment element is a balloon filled with cold fluid (e.g. ablative fluid at a sufficiently low temperature to cause tissue necrosis), the delivery zone can comprise all tissue surfaces contacted by the balloon that have heat directly extracted from them by the cold fluid (e.g. at a sufficient cold temperature to treat the tissue). In embodiments where the treatment element is an array of electrodes configured to deliver electrical energy (e.g. RF energy) to tissue, the delivery zone can comprise an area defined by the electrodes on the periphery of the array (e.g. a convex hull as described above), such as when the electrodes are positioned and energy is delivered to treat relatively the entire surface of tissue within the periphery. In embodiments where the treatment element comprises one or more fluid delivery elements delivering ablative fluid directly onto tissue (e.g. an ablative fluid whose chemical nature modifies tissue, at body temperature or otherwise), the delivery zone can comprise a surface defined by the periphery of tissue locations receiving the ablative fluid, such as when the ablative fluid is delivered (e.g. sprayed or otherwise applied, such as via a sponge) to relatively the entire surface within the periphery. In embodiments where the treatment element comprises one or more light delivery elements such as those that deliver laser energy to tissue, the delivery zone can comprise a surface area defined by the periphery of tissue locations receiving the light energy, such as when light is delivered at a set of locations and with a magnitude of energy configured to treat relatively the entire surface of tissue within the periphery. In these embodiments, light can be delivered to relatively the entire energy delivery zone, or to a large number (e.g. greater than 100) of tissue locations within the periphery of the delivery zone (e.g. making up less than 50%, less than 20% or less than 10% of the total surface area of the delivery zone). In embodiments where the treatment element comprises one or more sound delivery elements such as those that deliver sub-sonic and/or ultrasonic sound energy to tissue, the delivery zone can comprise a surface area defined by the periphery of tissue locations receiving the sound energy, such as when ablative sound energy is delivered at a set of locations and with a magnitude of energy configured to treat relatively the entire surface of tissue within the periphery. In embodiments in which the treatment element comprises a mechanical cutter or other abrasion element, the delivery zone can comprise a surface defined by all tissue dissected, cut, mechanically disrupted and/or otherwise modified during a single abrading step of the mechanical abrader.

A delivery zone can comprise a cumulative set of delivery zones that receive treatment simultaneously and/or sequentially, by one or more tissue treatment elements, such as those described herein. A delivery zone can comprise a first delivery zone defined when a treatment element treats target tissue in a first treatment delivery, plus a second delivery zone defined when the treatment element treats target tissue in a second treatment delivery, and so on. In these embodiments, the treatment element can be translated, rotated and/or otherwise repositioned between treatments (e.g. energy delivery), where each delivery zone is associated with the position of the treatment element during each treatment. Multiple delivery zones can receive treatment in a single procedure, such as within a period of less than twenty-four hours. A delivery zone can comprise a set of multiple delivery zones treated by two or more treatment elements.

Target tissue treated by each energy delivery and/or other treatment delivery comprises the tissue directly receiving treatment (i.e. the tissue defined by the delivery zone) plus “neighboring tissue” which is also modified by the associated treatment delivery. The neighboring tissue can comprise tissue alongside, below (e.g. in a deeper tissue layer) and/or otherwise proximate the delivery zone tissue. The neighboring tissue treatment can be due to one or more of: conduction and/or convection of heat or cold from the delivery zone; flow of ablative fluid from the delivery zone; flow of toxins or other agents that occur during cell degradation and/or cell death; radiation; luminescence, light dissipation; and other energy and/or chemical propagation mechanisms. In some embodiments, an area (i.e. the delivery zone) comprising an inner surface of mucosal tissue directly receives treatment from one or more treatment elements (e.g. an ablative fluid contained within a balloon), and the total volume of target tissue treated by that single treatment delivery includes: the delivery zone tissue (i.e. surface mucosal tissue directly receiving energy and/or other treatment from the treatment element); surface mucosal tissue in close proximity (e.g. adjacent) to the delivery zone tissue; and mucosal and potentially submucosal tissue layers beneath (deeper than) the delivery zone tissue and the treated adjacent surface mucosal tissue.

In some embodiments, a “treatment neutralizing” procedure is performed after one or more treatments (e.g. energy deliveries), such as a treatment neutralizing cooling procedure performed after one or more treatment elements deliver heat to treat target tissue, or a treatment neutralizing warming procedure performed after one or more treatment elements deliver cryogenic energy to treat target tissue. In these embodiments, the treatment neutralizing cooling or warming fluid can be delivered to the same functional assembly (e.g. an expandable functional assembly comprising a balloon) delivering the heat or cryogenic treatment, respectively, and/or the neutralizing fluid can be delivered directly to tissue by the same or different functional assembly or functional element. In some embodiments, a functional element delivers an ablating agent to target tissue (e.g. a chemical or other agent configured to cause target tissue necrosis or otherwise treat target tissue), and a treatment neutralizing procedure comprises delivery of a neutralizing agent (by the same or different functional element) to target and/or non-target tissue to reduce continued ablation due to the delivered caustic ablative fluid (e.g. a base to neutralize a delivered acid or an acid to neutralize a delivered base).

Each functional assembly and/or functional element of the present inventive concepts can be configured to be positioned in one or more intestinal and/or other locations of the patient, such as to perform a function (e.g. perform a treatment, deliver fluid and/or record data) at one or more contiguous or discontiguous tissue locations. Target tissue to be treated (e.g. ablated) comprises a three dimensional volume of tissue, and can include a first portion, a treatment portion, whose treatment has a therapeutic benefit to a patient; as well as a second portion, a “safety-margin” portion, whose treatment has minimal or no adverse effects to the patient. “Non-target tissue” can be identified (e.g. prior to and/or during the medical procedure), wherein the non-target tissue comprises tissue whose treatment by the treatment assembly and/or treatment element should be reduced or avoided such as to reduce or prevent an undesired effect to the patient.

The target tissue treatment can cause one or more modifications of the target tissue such as a modification selected from the group consisting of modification of cellular function; cell death; apoptosis; instant cell death; cell necrosis; denaturing of cells; removal of cells; and combinations of two or more of these. In some embodiments, the target tissue treatment is configured to create scar tissue. Target tissue can be selected such that after treatment the treated target tissue and/or the tissue that replaces the target tissue functions differently than the pre-treated target tissue, such as to have a therapeutic benefit for the patient. The modified and/or replacement tissue (singly or collectively “treated tissue”) can exhibit different properties than the pre-treated target tissue, such as different properties that are used to treat a patient disease or disorder. The treated tissue can have different secretions and/or quantities of secretions than the pre-treated target tissue, such as to treat diabetes, hypercholesterolemia and/or another patient disease or disorder. The treated tissue can have different absorptive properties than the target tissue, such as to treat diabetes, hypercholesterolemia and/or another patient disease or disorder. The treated tissue can have a different surface topography than the target tissue, such as a modification of the topography of the inner wall of the GI tract that includes a smoothing or flattening of its inner surface, such as a modification in which the luminal surface area of one or more segments of the GI tract is reduced after treatment. The effect of the treatment (e.g. the effect on the target tissue) can occur acutely, such as within twenty-four hours, or after longer periods of time, such as greater than twenty-four hours or greater than one week.

Target tissue to be treated can comprise two or more discrete tissue segments, such as two or more axial segments of the GI tract. Each tissue segment can comprise a full (e.g. approximately 360°) or partial circumferential segment of the tissue segment. Multiple tissue segments can be treated with the same or different functional elements (e.g. treatment elements), and they can be treated simultaneously or in sequential steps (e.g. sequential energy delivery steps that deliver energy to multiple delivery zones). Multiple tissue segments can be treated in the same or different clinical procedures (e.g. procedures performed on different days). In some embodiments, a series of tissue segments comprising a series of axial segments of the GI tract are treated in a single clinical procedure. The first and second tissue segments can be directly adjacent, they can contain overlapping portions of tissue, and/or there can be gaps between the segments. Dissimilarities in treatment elements can include type and/or amount of energy to be delivered by an energy delivery-based treatment element. Dissimilarities in target tissue treatments can include: target tissue area treated; target tissue volume treated; target tissue length treated; target tissue depth treated; target tissue circumferential portion treated; ablative fluid type, volume and/or temperature delivered to a reservoir such as a balloon; ablative fluid type, volume and/or temperature delivered directly to tissue; energy delivery type; energy delivery rate and/or amount; peak energy delivered; average temperature of target tissue achieved during target tissue treatment; maximum temperature achieved during target tissue treatment; temperature profile of target tissue treatment; duration of target tissue treatment; surface area reduction achieved by target tissue treatment; and combinations of two or more of these.

Target tissue can include tissue of the duodenum, such as tissue including substantially all or a portion of the mucosal layer of one or more axial segments of the duodenum (e.g. including all or a portion of the plicae circulares), such as to treat diabetes, hypercholesterolemia and/or another patient disease or disorder, such as while leaving the duodenum anatomically connected after treatment. Target tissue can include one or more portions of a tissue layer selected from the group consisting of: mucosa (e.g. including the stem cells at the base of the crypts); mucosa through superficial submucosa; mucosa through mid-submucosa; mucosa through deep-submucosa; and combinations of two or more of these. Replacement tissue can comprise cells that have migrated from one or more of: gastric mucosa; jejunal mucosa; an untreated portion of the duodenum whose mucosal tissue functions differently than the treated mucosal tissue functions prior to treatment; and combinations of two or more of these. Replacement tissue can include one or more tissue types selected from the group consisting of: scar tissue; normal intestinal mucosa; gastric mucosa; and combinations of two or more of these. In some embodiments, replacement tissue comprises tissue that has been delivered onto and/or into tissue by a catheter of the present inventive concepts. In some embodiments, target tissue includes a treatment portion comprising the mucosal layer of the duodenum, and a safety-margin portion comprising a near-full or partial layer of the submucosal layer of the duodenum. In some embodiments, the target tissue comprises nearly the entire mucosal layer of the duodenum, and this tissue can include a portion of the pylorus contiguous with the duodenal mucosa and/or a portion of the jejunum contiguous with the duodenal mucosa. In some embodiments, the target tissue comprises all or a portion of the duodenal mucosa distal to the ampulla of Vater (e.g. avoiding tissue within at least 0.5 cm, 1.0 cm or 1.5 cm from the ampulla of Vater while including tissue within 5 cm, 10 cm or 15 cm distal to the ampulla of Vater). In these embodiments, the target tissue can comprise at least 10%, at least 15%, at least 25%, at least 30% or at least 50% of the duodenal mucosa distal to the ampulla of Vater. Alternatively or additionally, the target tissue can comprise no more than 70% or no more than 90% of the duodenal mucosa distal to the ampulla of Vater. In these embodiments, tissue proximal to and/or proximate the ampulla of Vater can comprise non-target tissue (i.e. tissue whose treatment is avoided or at least reduced).

In some embodiments, the target tissue comprises at least a portion of duodenal mucosal tissue, and the systems, methods and devices of the present inventive concepts are configured to counteract duodenal mucosal changes that cause an intestinal hormonal impairment leading to insulin resistance in patients. In these embodiments, the therapy provided can improve the body's ability to process sugar and dramatically improve glycemic control for patients with insulin resistance and/or Type 2 diabetes. In some embodiments, target tissue is treated to prevent and/or reduce cognitive decline (e.g. Alzheimer's Disease), such as by improving sugar metabolism in the brain, overcoming insulin resistance in the brain, reducing toxicity of beta amyloid, reducing oxidative stress, and/or reducing inflammation in the brain associated with neuronal death. In some embodiments, target tissue is treated to: prevent liver fibrosis and/or cirrhosis (e.g. non-alcoholic fatty liver disease NAFLD or non-alcoholic steatohepatitis NASH); reduce liver fat; reduce oxidative stress; and/or reduce inflammation in the liver associated with liver fibrosis and toxicity.

Hormones released from the intestinal mucosa play an important role in modulating glucose homeostasis, and different axial segments of the intestinal mucosa release different hormones in the fasting and post-prandial state, in order to modulate blood glucose in the fasting and post-prandial states, respectively. After a meal, the proximal intestinal mucosa senses the intestine for ingested glucose and releases (e.g. increases the secretion of) a collection of hormones in response to this signal. These hormones initiate (e.g. stimulate) the process of insulin release into the bloodstream after a meal, but they also induce some insulin resistance to prevent the released insulin from causing hypoglycemia before the body has a chance to absorb the ingested glucose. One such hormone that plays a role in this is GIP. Distal gut hormones (produced in the jejunum or a more distal location), on the contrary, allow the release of more insulin but also play a role in helping the body now become sensitive to its circulating insulin. Teleologically, the explanation for this difference in the type of gut hormones produced by different segments of the intestine is that enough glucose will have been absorbed by the time nutrients reach the distal intestine to allow the insulin to begin to function to reduce blood glucose levels. Releasing different hormones at different times (e.g. from different segments of the intestine) enables the body to absorb and process glucose in such a way as to avoid hypoglycemia (blood sugars that are too low) and hyperglycemia (blood sugars that are too high). In this way, intestinal hormonal signaling is important for whole body glucose homeostasis in the fasting and post-prandial states. The treatment can also lead to weight loss through decreased absorption of nutrients, increased sensation of satiety, altered food preferences, increased energy expenditure, and combinations of two or more of these.

In patients with Type 2 diabetes, a lifetime of exposure to fat and sugar can lead to intestinal changes that occur in regions with the highest exposure to these nutrients, predominantly in the proximal intestine. These changes are characterized by an excess proximal intestinal mucosa's hormonal contribution to the fasting and post-prandial glucose homeostasis. The net result of these intestinal changes is to create a condition of insulin resistance and impaired glucose tolerance. Treatment of duodenal mucosal tissue with the systems, devices and methods of the present inventive concepts can be performed to alter the intestinal mucosal hormone production from the region of treated tissue. The treated tissue can then have an altered hormonal secretion pattern that affects blood glucose levels in the fasting and post-prandial states. The tissue treatment of the present inventive concepts can be performed to effect duodenal mucosal tissue secretion of GIP and/or GLP-1. The tissue treatment can lead to changes in the blood levels of GIP and/or GLP-1 (and other gut hormones) that can lead to changes in glucose homeostasis in the fasting and/or post-prandial states. The treatment can lead to changes in insulin and/or glucagon secretion from the pancreas and/or insulin and/or glucagon levels in the bloodstream. The treatment can lead to changes in pancreatic beta cell function and/or health through direct hormonal consequences of the treated duodenal tissue and/or indirectly through improved blood glucose levels. In some embodiments, the treatment of the present inventive concepts is configured to at least one of reduce a blood glucose level and/or reduce a lipoprotein level.

Treatment of intestinal tissue (e.g. duodenal mucosal tissue) using the systems, devices, and methods of the present inventive concepts can be performed to treat a medical condition (e.g. a disease and/or disorder) selected from the group consisting of: diabetes; pre-diabetes; impaired glucose tolerance; insulin resistance; a condition caused by or otherwise related to insulin resistance; obesity or otherwise being overweight; a metabolic disorder and/or disease; a condition caused by or otherwise related to a metabolic disorder and/or disease; and combinations of two or more of these. In some embodiments, treatment of intestinal tissue (e.g. at least duodenal mucosal tissue) using the systems, devices and/or methods of the present inventive concepts can be performed to treat one or more medical conditions selected from the group consisting of: Type 2 diabetes; Type 1 diabetes; “Double diabetes”; gestational diabetes; hyperglycemia; pre-diabetes; impaired glucose tolerance; insulin resistance; non-alcoholic fatty liver disease (NAFLD); non-alcoholic steatohepatitis (NASH); obesity; obesity-related disorder; polycystic ovarian syndrome (PCOS); hypertriglyceridemia; hypercholesterolemia; psoriasis; GERD; coronary artery disease (e.g. as a secondary prevention); stroke; TIA; cognitive decline; dementia; Alzheimer's disease; neuropathy; diabetic nephropathy; retinopathy; heart disease; diabetic heart disease; heart failure; diabetic heart failure; hirsutism; hyperandrogenism; fertility issues; menstrual dysfunction; cancer such as liver cancer, ovarian cancer, breast cancer, endometrial cancer, cholangiocarcinoma, adenocarcinoma, glandular tissue tumor(s), stomach cancer, large bowel cancer, and/or prostate cancer; diastolic dysfunction; hypertension; myocardial infarction; microvascular disease related to diabetes; sleep apnea; arthritis; rheumatoid arthritis; hypogonadism; insufficient total testosterone levels; insufficient free testosterone levels; and combinations of two or more of these. In some embodiments, two, three, or more of the above medical conditions listed immediately hereabove are treated using the systems, devices, and methods of the present inventive concepts. A near full circumferential portion (e.g. approximately 360°) of the mucosal layer of one or more axial segments of GI tissue can be treated. In some embodiments, less than 360° of one or more axial segments of tubular tissue is treated, such as one or more circumferential portions less than 350°, or between 300° and 350°, such as to prevent a full circumferential scar from being created at the one or more axial segment locations. In order to achieve a desired therapeutic benefit, a minimum amount of mucosal tissue can be treated, such as is described herein.

In some embodiments, the systems, devices, and methods of the present inventive concepts are used to treat arthritis, such as rheumatoid arthritis. In these embodiments, arthritis and another disease or disorder of the patient can be treated, such as when one, two, or more of the following are treated in addition to arthritis: insulin resistance, diabetes, non-alcoholic fatty liver disease (NAFLD); non-alcoholic steatohepatitis (NASH); polycystic ovarian syndrome (PCOS); and combinations of these. For example, patients with arthritis may exhibit abnormal and/or dysfunctional glucose metabolism. In some embodiments, a patient exhibiting insulin resistance as well as arthritis (e.g. rheumatoid arthritis) has their small intestinal mucosa (e.g. their duodenal mucosa) treated with the systems of the present inventive concepts.

Target tissue can be selected to treat two or more patient diseases or disorders, such as two or more patient diseases or disorders as described herein.

Target tissue can comprise tissue of the terminal ileum, such as to treat hypercholesterolemia and/or diabetes. In these embodiments, the target tissue can extend into the proximal ileum and/or the colon.

Target tissue can comprise gastric mucosal tissue, such as tissue regions that produce ghrelin and/or other appetite regulating hormones, such as to treat obesity and/or an appetite disorder.

Target tissue can comprise tissue selected from the group consisting of: large and/or flat colonic polyps; margin tissue remaining after a polypectomy; and combinations of two or more of these. These tissue locations can be treated to treat residual cancer cells.

Target tissue can comprise at least a portion of the intestinal tract afflicted with inflammatory bowel disease, such that Crohn's disease and/or ulcerative colitis can be treated.

Target tissue can comprise GI tissue selected to treat Celiac disease and/or to improve intestinal barrier function.

The functional assemblies, functional elements, systems, devices and methods of the present inventive concepts can be configured to avoid ablating or otherwise adversely affecting certain tissue, termed “non-target tissue” herein. Depending on the location of tissue intended for treatment (i.e. target tissue), different non-target tissue can be applicable. In certain embodiments, non-target tissue can comprise tissue selected from the group consisting of: gastrointestinal adventitia; duodenal adventitia; the tunica serosa; the tunica muscularis; the outermost partial layer of the submucosa; ampulla of Vater; the papilla; the pre-papillary duodenum; pancreas; bile duct; pylorus; and combinations of two or more of these.

In some embodiments, two or more clinical procedures are performed in which one or more volumes of target tissue are treated in each clinical procedure, such as is described in applicant's co-pending U.S. patent application Ser. No. 14/673,565 (Attorney Docket No. 41714-708.301; Client Docket No. MCT-009-US), entitled “Methods, Systems and Devices for Performing Multiple Treatments on a Patient”, filed Mar. 30, 2015. For example, a second clinical procedure can be performed at least twenty-four hours after the first clinical procedure, such as a second clinical procedure performed within six months of a first clinical procedure or a clinical procedure performed after at least six months after the first clinical procedure. The first and second clinical procedures can be performed using similar or dissimilar methods, and they can be performed using similar or dissimilar systems and/or devices (e.g. performed with similar or dissimilar treatment and/or other functional elements). The first and second clinical procedures can treat similar or dissimilar volumes of target tissue (e.g. similar or dissimilar amounts of tissue treated and/or locations of tissue treated), and they can deliver energy to similar or dissimilar sets of multiple delivery zones. In some embodiments, the first and second clinical procedures can include treating and/or delivering energy to contiguous and/or overlapping regions of the GI tract either in the circumferential and/or axial dimensions. In other embodiments, the first and second clinical procedures can include the treatment of disparate regions of the GI tract (such as disparate regions of the duodenum, ileum, and/or stomach). The first and second clinical procedures can be performed using similar or dissimilar devices (e.g. catheters). The first and second clinical procedures can comprise similar or dissimilar deliveries of energy to treat the target tissue. The first and second clinical procedures can be performed at similar or dissimilar temperatures. The second clinical procedure can be performed based on diagnostic results collected after the first clinical procedure has been performed, such as when the diagnostic results are based on a biopsy of mucosal tissue.

The functional assemblies, treatment assemblies, treatment elements and other functional elements of the present inventive concepts can comprise an expandable element or otherwise be configured to automatically and/or manually expand or traverse in at least one radial direction. Typical expandable elements include but are not limited to: an inflatable balloon; a radially expandable cage or stent; one or more radially deployable arms; an expandable helix; an unfurlable compacted coiled structure; an unfurlable sheet; an unfoldable compacted structure; and combinations of two or more of these. In some embodiments, an expandable element can comprise a radially expandable tube, such as a sheet of material resiliently biased in a radially expanded condition that can be compacted through a furling operation, or a sheet of material resiliently biased in a radially compact condition that can be expanded through an unfurling operation. An expandable element can comprise a foldable sheet, such as a sheet configured to be folded to be radially compacted and/or to be unfolded to radially expand. In some embodiments, an expandable element expands to contact tissue, such as to expand to a diameter similar to the diameter of the luminal wall tissue into which the expandable element has been placed. In some embodiments, an expandable element expands to be closer to wall tissue, but the element remains at a distance (e.g. a fixed or pre-determined distance) from the tissue surface, such as when the tissue is subsequently brought into contact with all or a portion of an expanded functional assembly or functional element (e.g. using insufflation fluid withdrawal techniques). In some embodiments, an expandable element expands to be larger than the diameter of the luminal wall tissue into which the expandable element has been placed, such as to improve the quality of the apposition of the expandable element against the uneven surface of the tissue. In these embodiments, the fully expanded diameter of an expandable element would be configured to avoid a diameter large enough to cause lasting mechanical damage to the apposed tissue and/or to tissue proximate the apposed tissue. In some embodiments, the expansion of an expandable element (e.g. the expansion of an expandable functional assembly) is monitored and/or varied (e.g. decreased and/or increased), such as to accommodate or otherwise compensate for peristalsis or other muscle contractions that occur in the GI tract (e.g. contractions that occur when a foreign body is present in the GI tract) and/or varied to accommodate changes in GI lumen diameter imposed by aspects of the procedure itself.

Any device (e.g. catheter) of the present inventive concepts can include one or more functional elements comprising one or more treatment elements configured to deliver energy to one or more delivery zones, to treat at least a portion of target tissue. Any device can include one or more functional elements comprising one or more fluid delivery elements, such as one or more nozzles or needles configured to deliver fluid toward and/or into tissue. The fluid delivery elements can be constructed and arranged to deliver fluid to perform a function selected from the group consisting of: expanding one or more tissue layers; warming or cooling tissue; removing debris or other substance from a tissue surface; delivering energy to a delivery zone comprising a continuous or segmented surface; treating target tissue; and combinations of two or more of these. Any of the expandable functional assemblies of the present inventive concepts can include one or more other functional elements, such as are described herein. The treatment elements and/or other functional elements (e.g. fluid delivery elements) can be mounted on, within (e.g. within the wall) and/or inside of an expandable element such as a balloon or expandable cage. In some embodiments, one or more functional elements is not mounted to an expandable element, such as those attached to a shaft or other non-expandable catheter component.

In some embodiments, a catheter comprises at least one functional element configured to deliver energy to a delivery zone such as to ablate target tissue. Examples of ablation-based functional elements include but are not limited to: ablative fluids, such as hot or cold ablative fluids delivered to a balloon and/or directly to target tissue; one or more fluid delivery elements configured to deliver ablative fluid directly to target tissue; an RF and/or microwave energy delivery element such as one or more electrodes; an ultrasonic and/or subsonic transducer such as one or more piezo crystals configured to ablate tissue with ultrasonic or subsonic energy, respectively, sound waves; a laser energy delivery element such as one or more optical fibers, laser diodes, prisms and/or lenses; a rotating ablation element; a circumferential array of ablation elements; and combinations of two or more of these.

The expandable elements comprising balloons of the present inventive concepts can be divided into two general categories: those that are composed of a substantially elastic material, such as silicone, latex, low-durometer polyurethane, and the like; and those that are composed of a substantially inelastic material, such as polyethylene terephthalate (PET), nylon, high-durometer polyurethane and the like. A third category includes balloons which include both elastic and inelastic portions. Within the category of elastic balloons, two subcategories exist: a first sub-category wherein a combination of material properties and/or wall thickness can be combined to produce a balloon that exhibits a measurable pressure-threshold for inflation (i.e. the balloon becomes inflated only after a minimum fluidic pressure is applied to the interior of the balloon); and a second sub-category, wherein the balloon expands elastically until an elastic limit is reached which effectively restricts the balloon diameter to a maximum value. The individual properties of the balloons in each of these categories can be applied to one or more advantages in the specific embodiments disclosed herein, these properties integrated singly or in combination. By way of example only, one or more of the following configurations can be employed: a highly elastic balloon can be used to achieve a wide range of operating diameters during treatment (e.g. during operation a desired balloon diameter can be achieved by adjustment of a combination of fluid temperature and pressure); a substantially inelastic balloon or a balloon that reaches its elastic limit within a diameter approximating a target tissue diameter (e.g. a duodenal mucosal diameter) can be used to achieve a relatively constant operating diameter that will be substantially independent of operating pressure and temperature; a balloon with a pressure-threshold for inflation can be used to maintain an uninflated diameter during relatively low pressure conditions of fluid flow and then achieve a larger operating diameter at higher pressure conditions of flow. Pressure-thresholded balloons can be configured in numerous ways. In one embodiment, a balloon is configured to have a relatively thick wall in its uninflated state, such as to maximize an electrically and/or thermally insulating effect while the balloon is maintained in this uninflated state. The balloon can be further configured such that its wall thickness decreases during radial expansion (e.g. to decrease an electrically and/or thermally insulating effect). In another embodiment, a balloon is configured to have a relatively small diameter in its uninflated state (e.g. a diameter that is small relative to the inner diameter of tubular target tissue such as the diameter of the mucosal layer of duodenal wall tissue), such as to minimize or completely eliminate apposition between the balloon and the surrounding tissue to minimize heat, RF and/or other energy transfer into the surrounding tissue until the balloon is fully inflated. In another embodiment, a balloon and an ablation system or catheter are configured to circulate a flow of fluid through the balloon (e.g. an elastic balloon or an inelastic balloon) at a sufficiently low enough pressure to prevent apposition of the balloon or other catheter component with target tissue, such as to pre-heat one or more surfaces of the ablation system or ablation device that are in fluid communication with the balloon. In this configuration, when the balloon or other ablation element is positioned to deliver energy to target tissue, the temperature of the balloon or other ablation element will be at a desired level or it will rapidly and efficiently reach the desired level for treatment (i.e. minimal heat loss to the fluid path components due to the pre-heating or pre-cooling). These configurations provide a method of delivering energy to tissue with an ablative fluid filled balloon. A “thermal priming” procedure can be performed prior to one or more target tissue treatments, such as to improve thermal response time of one or more portions of the catheter. Ablative fluid filled balloon catheters as well as thermal priming devices and methods can be configured as is described in applicant's co-pending U.S. patent application Ser. No. 16/438,362 (Attorney Docket No. 41714-704.302; Client Docket No. MCT-002-US-CON1), entitled “Heat Ablation Systems, Devices and Methods for the Treatment of Tissue”, filed Jun. 11, 2019, the content of which is incorporated herein by reference in its entirety for all purposes.

A fluid evacuation procedure can be performed on one or more internal locations of the catheters, functional assemblies and/or functional elements of the present inventive concepts, such as when a negative pressure is applied to purge or otherwise evacuate fluid from one or more locations. A fluid evacuation procedure can be performed prior to a thermal priming procedure and/or prior to delivering ablative fluid to a treatment element.

At times during target tissue treatment when it is desirable to initiate, increase and/or otherwise modify the treatment of tissue by one or more treatment elements (e.g. a fluid delivery element delivering ablative fluid, a mechanically abrasive element, a hot or cold fluid balloon delivering a thermal energy to tissue and/or an electrode delivering RF energy), the diameter of the treatment assembly and/or treatment element (e.g. the diameter of a balloon, deployable cage, expandable tube or other expandable assembly) can be increased in situ to move a treatment element closer to target tissue and/or to change the contact force between the treatment element and the target tissue. At times during treatment when it is desirable to stop or otherwise decrease the amount of tissue treatment, the diameter of the treatment assembly and/or treatment element can be reduced in situ, such as to prevent or otherwise reduce delivery of energy or other treatment to the target tissue by eliminating or reducing tissue contact of one or more treatment elements (e.g. electrodes, abrasive surfaces or ablative fluid-filled balloons). For those cases where the native diameter of the target tissue varies substantially within a delivery zone, then a highly elastic or compliant balloon or other expandable element can be employed, such as a balloon or deployable cage which can be adjusted to achieve a wide range of operating diameters.

Alternatively or additionally, to initiate, increase and/or otherwise modify the treatment of tissue by one or more functional elements (e.g. a fluid delivery element delivering ablative fluid, a mechanically abrasive element, a hot or cold fluid balloon delivering thermal energy to or from tissue and/or an electrode delivering RF energy), the diameter of the target tissue can be decreased in situ to move target tissue closer to a treatment element and/or to change the contact force between the target tissue and the treatment element. To stop or otherwise decrease ablation of tissue, the diameter of tissue neighboring a treatment element can be increased in situ, such as to prevent or otherwise reduce delivery of energy or other treatment to the target tissue by eliminating or reducing tissue contact of one or more treatment elements (e.g. electrodes, abrasive surfaces or ablative fluid filled balloons). The diameter of the tissue proximate a functional assembly can be increased or decreased, independent of the functional assembly diameter, by means of delivering and/or withdrawing a fluid, to and/or from a body lumen (e.g. a lumen of a segment of the intestine) surrounded by target tissue, such as by using standard GI insufflation techniques. Typical insufflation fluids include but are not limited to: gases such as carbon dioxide or air; liquids such as water or saline solution; and combinations of two or more of these. The insufflation fluids can be introduced through a catheter, through an endoscope such as an endoscope through which the catheter is inserted, and/or via another device placed proximate the target tissue. Delivery of insufflation fluids can be performed to move target tissue away from one or more functional elements, such as to stop transfer of energy to target tissue at the end of a treatment of target tissue as described hereabove. Alternatively or additionally, delivery of insufflation fluids can be performed to manipulate tissue, such as to distend and/or elongate tissue. Extraction of these insufflation fluids and/or the application of a vacuum or other negative pressure can be used to decrease the diameter of the target tissue, such as to bring the target tissue in closer proximity to one or more functional elements and/or to increase the contact force between target tissue and one or more functional elements, also as described hereabove. In this tissue diameter-controlled approach, a functional assembly including a balloon that can be maintained at a substantially constant diameter can be desirable, such as a substantially inelastic balloon such as a balloon with an elastic-limit.

The systems of the present inventive concepts can include one or more tissue expansion catheters that comprise one or more functional elements configured as fluid delivery elements. In these embodiments, the one or more functional elements can comprise one or more needles, nozzles and/or fluid jets configured to deliver one or more fluids or other injectates to tissue, such as to expand target tissue and/or tissue proximate the target tissue (e.g. safety margin tissue) prior to treatment of target tissue by a tissue treatment element. The expanded tissue layer acts as a safety volume of tissue, reducing the specificity of the treatment (e.g. ablation) required and/or the need to protect the underlying non-target tissue from damage. In some embodiments, a vacuum pressure can be used to manipulate tissue and/or to maintain proximity between a portion of a tissue expansion device and tissue. The vacuum can be provided by one or more vacuum sources, such as via one or more operator adjustable vacuum sources.

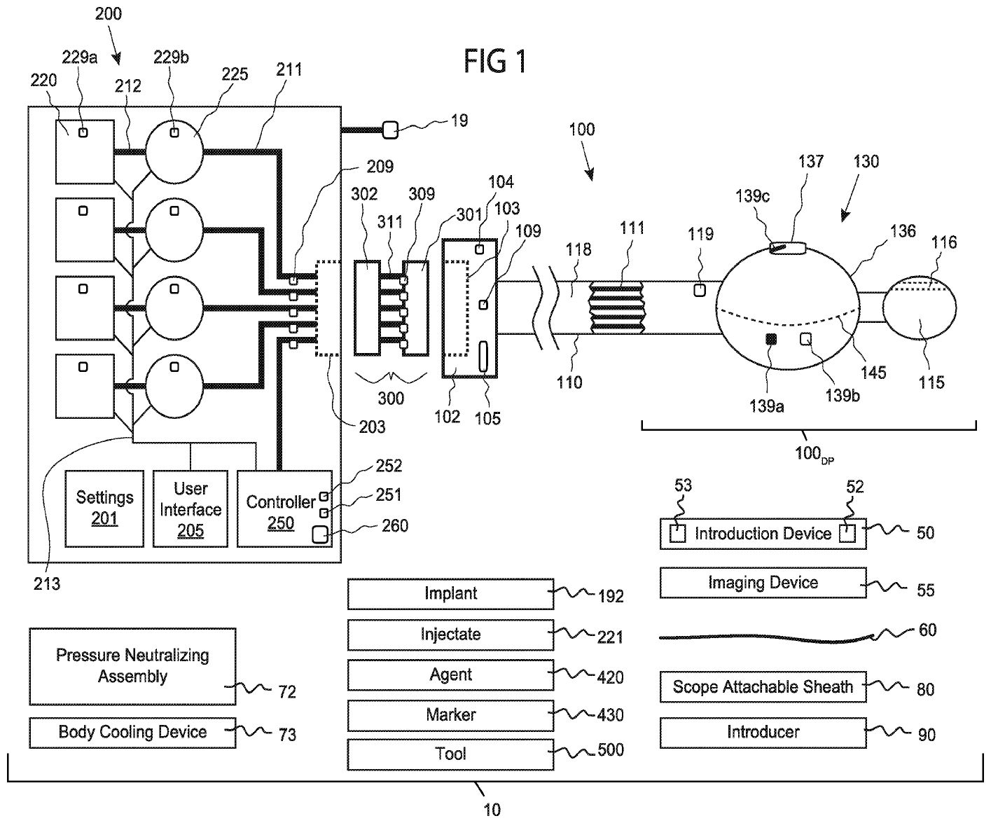

Referring now to FIG. 1, a schematic view of a system for performing a medical procedure on a patient is illustrated, consistent with the present inventive concepts. The medical procedure can comprise a diagnostic procedure (e.g. a diagnostic and/or prognostic procedure), a therapeutic procedure, or a combined diagnostic and therapeutic procedure. System 10 comprises one or more treatment devices, catheter 100, (e.g. a catheter, flexible probe, and/or other elongate treatment device for insertion into a patient), and a console, console 200, which operably attaches to the one or more catheters 100 (e.g. attaches to two, three or more catheters 100). Catheter 100 comprises an elongate shaft, shaft 110, comprising one or more shafts (e.g. shafts with flexible and/or rigid segments). In some embodiments, shaft 110 comprises multiple shafts in a spiraled configuration (e.g. helical configuration).

Catheter 100 comprises one or more functional assemblies, assembly 130 shown, which can be configured to radially expand and/or contract. Functional assembly 130 can be positioned on a distal portion of catheter 100 (e.g. on the distal end or a distal portion of shaft 110), distal portion 100_{DP}. In some embodiments, functional assembly 130 comprises a non-circular cross section (e.g. to “hug” a second device such as an endoscope simultaneously inserted into the patient). Functional assembly 130 can comprise one or more tissue-contacting portions, as described hereabove (e.g. side walls of functional assembly 130 that contact inner wall tissue of the intestine or other GI lumen). Functional assembly 130 can comprise a tissue-contacting surface area (e.g. when expanded) of between 500 mm² to 3500 mm², such as a tissue contacting surface area of approximately between 1000 mm² and 2000 mm², or approximately between 1250 mm² and 1750 mm², or approximately 1500 mm². In some embodiments, functional assembly 130 comprises an expanded diameter of at least 18 mm, and/or no more than 30 mm, such as an expanded diameter of approximately 19 mm, 22 mm, 25 mm or 28 mm. In some embodiments, functional assembly 130 comprises a tissue-contacting length (e.g. when expanded) of between 10 mm and 40 mm, such as a length of approximately 15 mm, 20 mm, 25 mm or 30 mm. This tissue-contacting length represents the “treatment length” of the functional assembly 130. In some embodiments, system 10 includes a first catheter 100 comprising a functional assembly 130a, and a second catheter 100 comprising a functional assembly 130b. Functional assemblies 130a and 130b can be similar or different, such as when a functional assembly 130a and 130b have different geometries (e.g. different lengths, expanded diameters; and/or tissue contacting surface areas), deliver different treatments (e.g. deliver different forms of ablative energies and/or deliver different ablative fluids), and/or perform different neutralizing procedures.

In some embodiments, shaft 110 passes through all or a portion of functional assembly 130. In other embodiments, functional assembly 130 is positioned on a distal end of shaft 110. In some embodiments, shaft 110 comprises a non-circular cross section (e.g. to “hug” a second device such as an endoscope simultaneously inserted into the patient). In some embodiments, shaft 110 comprises one or more of: a braided portion; a tapered portion; an insertable stiffening mandrel; a variable stiffness portion; and combinations of two or more of these.

In some embodiments, functional assembly 130 comprises one or more biasing members, such as biasing member 145 shown. Biasing member 145 is constructed and arranged to apply a force to functional assembly 130, such as to place functional assembly 130 in tension along the axis of shaft 110 proximate functional assembly 130, such as when functional assembly 130 is in an unexpanded state. Biasing member 145 can be constructed and arranged to bend as functional assembly 130 expands. Biasing member 145 can comprise an element selected from the group consisting of: spring; coil spring; leaf spring; flexible filament; flexible sheet; nickel titanium alloy component; and combinations of two or more of these. In some embodiments, functional assembly 130 comprises balloon 136, and biasing member 145 is configured to avoid contacting balloon 136 when functional assembly is in its unexpanded state.

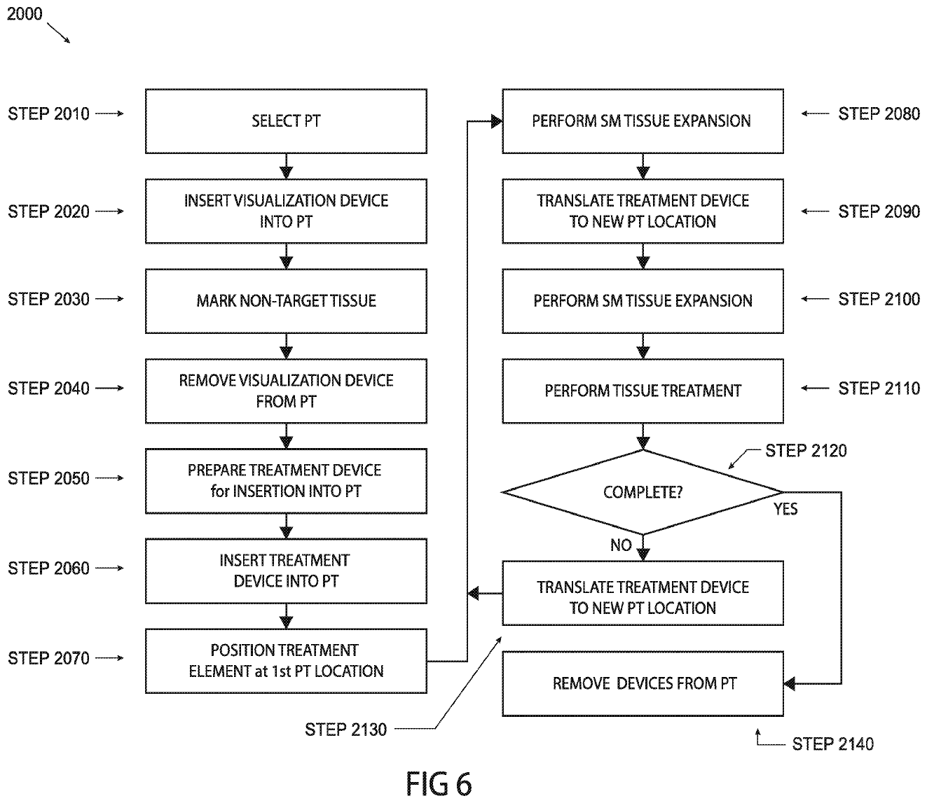

In some embodiments, functional assembly 130 comprises a shape constructed and arranged to prevent or otherwise reduce migration of functional assembly 130. In some embodiments, functional assembly 130 is constructed and arranged to perform a first procedure (e.g. a tissue expansion procedure), anchor in tissue (e.g. anchoring performed prior to the first procedure, during the first procedure and/or after the first procedure) and subsequently perform a second procedure (e.g. a tissue ablation procedure), such as is described herebelow in reference to FIG. 6.

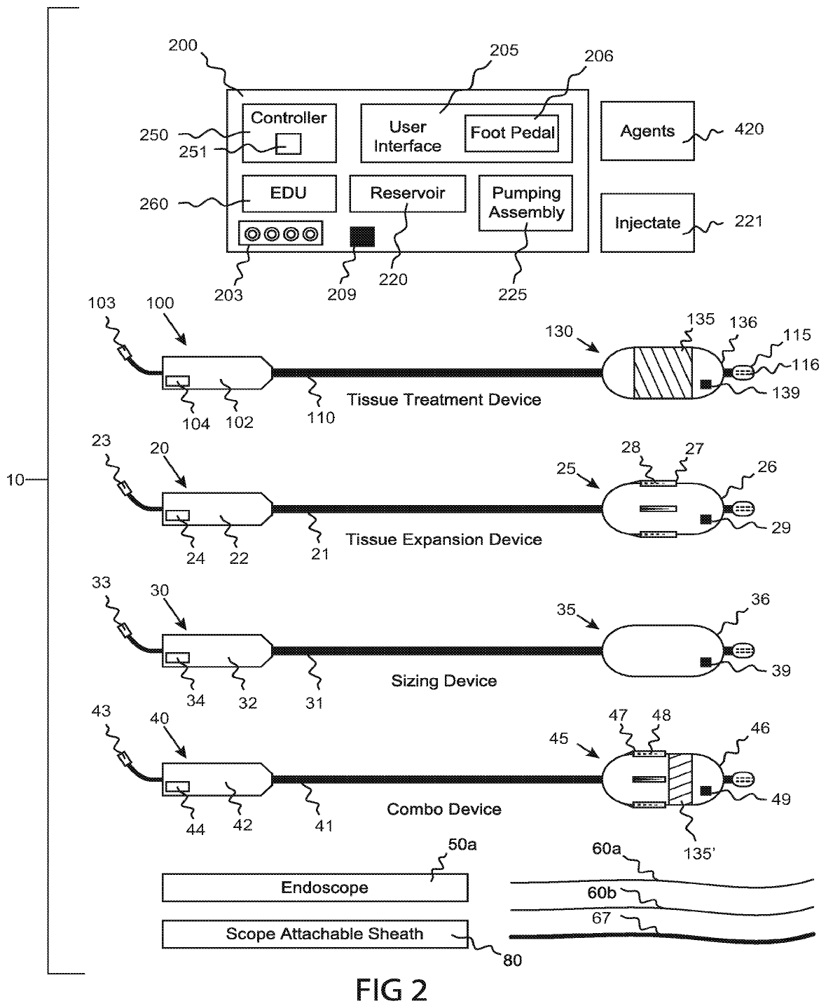

Catheter 100 can comprise one or more catheters of similar construction and arrangement (e.g. and include similar components) as one or more of catheters 100, 20, 30 and/or 40 of FIG. 2, each described in detail herebelow. Catheter 100 can be constructed and arranged to perform a medical procedure in an intestine of the patient, such as a procedure in the small intestine (e.g. in the duodenum) and/or in the large intestine. In some embodiments, system 10 further comprises a connecting assembly, assembly 300, which can be constructed and arranged to operably attach (e.g. fluidly, mechanically, electrically and/or optically connect) catheter 100 to console 200. In alternate embodiments, catheter 100 can operably attach directly to console 200, without connecting assembly 300. Console 200 can be of similar construction and arrangement as console 200 of FIG. 2, also described in detail herebelow.

System 10 can further comprise body introduction device 50, guidewire 60, sheath 80 (e.g. an endoscope-attachable sheath), introducer 90 (e.g. an introducer sheath), injectate 221, and/or agent 420, each of which can be of similar construction and arrangement to the similar components described in detail herebelow in reference to FIG. 2. In some embodiments, guidewire 60 comprises two or more guidewires. Body introduction device 50 can comprise one or more: endoscopes; laparoscopic ports; and/or vascular introducers. Body introduction device 50 can comprise a camera, such as camera 52 shown, and a display, not shown but such as a display of console 200 and/or another display used to display an image (e.g. a camera view) provided by camera 52. In some embodiments, device 50 comprises an endoscope and includes a cap, scope cap 53 shown, which is attached (or attachable) to a distal end of the endoscope, such as to limit tissue collapse that would limit visualization provided by camera 52. Scope cap 53 can extend between 2-6 mm in front of camera 52. In some embodiments, scope cap 53 is of similar construction and arrangement to the Reveal® distal attachment cap manufactured by US Endoscopy.

In some embodiments, system 10 further comprises imaging device 55, which can comprise an imaging device constructed and arranged to provide an image of the patient's anatomy (e.g. inner wall or any part of the intestine of the patient) and/or an image of all or part of catheter 100 or other portion of system 10, as described in detail herein. Imaging device 55 can comprise an imaging device selected from the group consisting of: endoscope camera; visible light camera; infrared camera; X-ray imager; fluoroscope; Ct Scanner; MRI; PET Scanner; ultrasound imaging device; molecular imaging device; and combinations of two or more of these. In some embodiments, a patient image is used to set, confirm and/or adjust one or more system 10 parameters, such as when imaging device 55 comprises a sensor-based functional element configured to produce a signal.

In some embodiments, system 10 further comprises functional element 19 comprising a sensor, transducer, and/or other functional element. Functional element 19 can be operably attached to console 200 or another component of system 10. Functional element 19 can comprise a sensor configured to produce a signal, which can be used to modify a parameter of system 10, as described in detail herein. In some embodiments, functional element 19 comprises a sensor configured to measure a patient parameter, such as a patient parameter selected from the group consisting of: a patient physiologic parameter; blood pressure; heart rate; pulse distention; glucose level; blood glucose level; blood C-peptide level; blood glucagon level; blood insulin level; blood gas level; hormone level; GLP-1 level; GIP level; EEG; LFP; respiration rate; breath distention; perspiration rate; temperature; gastric emptying rate; peristaltic frequency; peristaltic amplitude; a patient anatomical parameter such as tissue geometry information; a patient environment parameter such as room pressure or room temperature; and combinations of two or more of these.

In some embodiments, system 10 further comprises one or more tools, such as a tool 500 described herebelow.

In some embodiments, system 10 comprises one or more sensors, such as when one or more functional elements of system 10 are configured as a sensor, such as functional elements 109, 119, 139, 229 and/or 309 described in detail herebelow. Each of the system 10 sensors can be configured to produce a signal related to a patient parameter and/or a system 10 parameter. For purposes herein, a signal “related” to a parameter shall include signals that directly represent the parameter, as well as signals that provide information that can be correlated to or in any way relate to the parameter. For example, a sensor (e.g. a temperature or pressure sensor) placed proximate tissue or a component of system 10 can directly represent a parameter (e.g. the temperature or pressure, respectively) of locations proximate that tissue or component, respectively. Alternatively, a sensor placed at one location (e.g. one location within system 10), can provide a signal that can be analyzed to produce information representing a parameter at a different location (e.g. a different location within system 10 or a location within the patient). For example, a temperature or pressure measured at one location (e.g. within console 200, connecting assembly 300 and/or a proximal portion of catheter 100) can correlate to a temperature or pressure, respectively, at a different location (e.g. proximate and/or within functional assembly 130). Correlation of signals provided by a sensor of system 10 to a parameter at a location distant from the sensor can be accomplished by one or more algorithms of system 10, such as algorithm 251 described herebelow.

In some embodiments, a system 10 sensor is configured to produce a signal related to an anatomic and/or physiologic parameter of the patient, such as a parameter selected from the group consisting of: a parameter of the intestine; a parameter related to the anatomical geometry of a portion of the intestine; a parameter related to force and/or pressure applied to tissue (e.g. tissue of the intestine); a parameter related to a pressure within tissue (e.g. tissue within the luminal surface of the intestine); a parameter related to temperature of tissue (e.g. tissue of the intestine); and combinations of two or more of these. In some embodiments, one or more sensors of system 10 comprise a camera configured to provide an image, and the signal provided by the sensor comprises the image and/or an analysis of the image. The signal provided by the sensor can relate to a patient parameter (e.g. a patient physiologic or anatomical parameter) or a system 10 parameter (e.g. a functional assembly 130 parameter).

In some embodiments, a system 10 sensor is configured to produce a signal related to a parameter of one or more components of system 10, such as a component of console 200, connecting assembly 300 and/or catheter 100. For example, the signal produced by one or more sensors of system 10 can be related to a functional assembly 130 parameter, such as a parameter selected from the group consisting of: pressure within functional assembly 130; force applied to and/or by a portion of functional assembly 130; temperature of at least a portion of functional assembly 130; temperature of fluid within functional assembly 130; state of expansion of functional assembly 130; position of functional assembly 130 (e.g. position of functional assembly 130 relative to the patient's anatomy): and combinations of two or more of these.

In some embodiments, system 10 is configured to perform a therapeutic procedure selected from the group consisting of: a tissue removal procedure such as a tissue removal procedure in which mucosal intestinal tissue is removed; a tissue ablation procedure such as a tissue ablation procedure in which at least intestinal mucosal tissue is removed; a tissue expansion procedure such as a tissue expansion procedure configured to create a safety margin of tissue (e.g. a safety margin comprising expanded submucosal tissue), and/or a tissue expansion procedure configured to create a therapeutic restriction; and combinations of two or more of these. In some embodiments, system 10 is configured to treat one or more medical conditions (e.g. diseases and/or disorders), such as are described hereabove. For example, system 10 can be configured to treat diabetes, such as Type 2 diabetes, Type 1 diabetes, “Double diabetes” and/or gestational diabetes. In some embodiments, system 10 is configured to treat hypercholesterolemia, such as when target tissue treated by functional assembly 130 includes tissue of the terminal ileum. In some embodiments, system 10 is configured to treat both diabetes and hypercholesterolemia. In some embodiments, system 10 is configured such that functional assembly 130 treats a part of the intestine exhibiting inflammatory bowel disease, ulcerative colitis and/or chronic ulcers. System 10 can be constructed and arranged to cause functional assembly 130 to expand one or more layers of tissue (e.g. submucosal tissue), and/or to treat target tissue (e.g. target tissue comprising mucosal tissue of the duodenum or other intestinal mucosa). System 10 can be further constructed and arranged to avoid adversely affecting non-target tissue, as described in detail herein and in applicant's co-pending U.S. patent application Ser. No. 15/917,480 (Attorney Docket No. 41714-703.302; Client Docket No. MCT-001-US-CON1), entitled “Devices and Methods for the Treatment of Tissue”, filed Mar. 9, 2018, the content of which is incorporated herein by reference in its entirety for all purposes.

In some embodiments, system 10 is constructed and arranged to alter intestinal microbiota, such as to perform a treatment that affects a patient's gut flora in a way that leads to an improvement in weight and/or metabolic status (e.g. to treat Type 2 diabetes). Catheter 100 and functional assembly 130 can be configured to treat target tissue including intestinal mucosa such as to destroy local bacteria and/or modify the microbiome in the treated tissue area. Target tissue can include tissue regions where the microbiota contributes to the incidence or maintenance of metabolic disease.

In some embodiments, system 10 is constructed and arranged to reduce or otherwise alter the surface area of intestinal mucosa, such as is described in applicant's co-pending U.S. patent application Ser. No. 14/956,710 (Attorney Docket No. 41714-709.302; Client Docket No. MCT-013-US-CON1), entitled “Methods, Systems and Devices for Reducing the Luminal Surface Area of the Gastrointestinal Tract”, filed Apr. 9, 2019, the content of which is incorporated herein by reference in its entirety for all purposes. In some embodiments, system 10 is configured to reduce or otherwise alter the surface area of intestinal mucosa as a treatment for diabetes, a metabolic disease, obesity and/or hypercholesterolemia. In these embodiments, treatment of target tissue comprising mucosal folds and/or other mucosal tissue results in intestinal mucosa with reduced plicae circulares and delayed recovery or regrowth of intestinal villi. The treatment provided by system 10 can comprise a durable treatment effect that reduces the total absorptive surface area of the treated region. Alternatively or additionally, the treatment provided by system 10 can reduce enteroendocrine cell and/or absorptive cell quantities in the intestine by reducing the geometric complexity of the intestinal surface, such as by a target tissue treatment comprising ablation of intestinal tissue to a certain depth (mucosa alone; mucosa and superficial submucosa; mucosa through mid-submucosa; or mucosa through deep submucosa) that induces the healing response that leads to elimination of plicae circulares and blunting of villi for a prolonged period of time (at least two weeks, at least six weeks, at least six months or at least one year).

In some embodiments, system 10 is configured to treat sufficient duodenal mucosa to provide an improvement in a patient's diabetes, such as is described in applicant's co-pending U.S. patent application Ser. No. 15/406,572 (Attorney Docket No. 41714-713.301; Client Docket No. MCT-029-US), entitled “Methods and Systems for Treating Diabetes and Related Diseases and Disorders”, filed Jan. 13, 2017, the content of which is incorporated herein by reference in its entirety for all purposes.

In some embodiments, system 10 is configured to treat NAFLD and/or NASH (“NAFLD/NASH” herein), such as is described in applicant's issued U.S. Pat. No. 9,757,535, entitled “Systems, Devices and Methods for Performing Medical Procedures in the Intestine”, filed Sep. 23, 2016, the content of which is incorporated herein by reference in its entirety for all purposes. In the embodiments, system 10 can be configured to treat patients inflicted with NAFLD/NASH, in addition to diabetes (e.g. Type 2 diabetes).

In some embodiments, system 10 is configured to create a therapeutic restriction in a patient, such as is described in applicant's co-pending U.S. patent application Ser. No. 16/267,771 (Attorney Docket No. 41714-711.302; Client Docket No. MCT-024-US-CON1) entitled “Systems, Devices and Methods for the Creation of a Therapeutic Restriction in the Gastrointestinal Tract”, filed Feb. 5, 2019, the content of which is incorporated herein by reference in its entirety for all purposes. In some embodiments, the therapeutic restriction is created at a location selected from the group consisting of: within mucosal tissue; within submucosal tissue; between mucosal and submucosal tissue; and combinations thereof. In some embodiments, the therapeutic restriction is created at a location selected from the group consisting of: lower stomach; pylorus; proximal small intestine; duodenum; proximal jejunum; distal small intestine; distal jejunum; ileum; and combinations thereof. In some embodiments, the therapeutic restriction is created in a location selected from the group consisting of: colon; rectum; anal sphincter and combinations thereof. In some embodiments, the therapeutic restriction is created by injecting (e.g. via one or more fluid delivery elements 139c) a volume of injectate, injectate 221, of at least 1.0 mL. The therapeutic restriction can be created by injecting a volume of injectate 221 of at least 3.0 mL, or at least 4.0 mL. In some embodiments, the therapeutic restriction is created by injecting a volume of injectate 221 of no more than 20.0 mL. The therapeutic restriction can be created by injecting a volume of injectate 221 of no more than 10.0 mL, or no more than 8.0 mL. In some embodiments, the therapeutic restriction comprises an axial length of at least 1 mm, or at least 5 mm, or at least 10 mm. In some embodiments, the therapeutic restriction can comprise an axial length of no more than 100 mm, no more than 50 mm, or no more than 20 mm. In some embodiments, the therapeutic restriction comprises an inner diameter (e.g. diameter of its open portion) that is less than or equal to 10 mm. The therapeutic restriction can comprise an inner diameter less than or equal to 5 mm, 4 mm, 3 mm, 2 mm or 1 mm. In some embodiments, the therapeutic restriction comprises an inner diameter that is between 1% and 50% (e.g. 99% to 50% narrowing, respectively) of the inner diameter of the luminal segment prior to creation of the therapeutic restriction. The therapeutic restriction can comprise an inner diameter that is between 1% and 20% of the inner diameter of the luminal segment prior to creation of the therapeutic restriction. The inner diameter of the therapeutic restriction can increase over time, such as via the therapeutic restriction volume decreasing over time such as via absorption, migration or other reduction of the delivered injectate 221. The inner diameter of the therapeutic restriction can increase to an inner diameter that is between 11% and 20% of the inner diameter of the luminal segment prior to creation of the therapeutic restriction. The therapeutic restriction can comprise an inner diameter that is between 1% and 10% of the inner diameter of the luminal segment prior to creation of the therapeutic restriction. The therapeutic restriction can comprise an inner diameter that is between 1% and 5% of the inner diameter of the luminal segment prior to creation of the therapeutic restriction.

System 10 can be constructed and arranged to perform one or more diagnostic procedures (e.g. one or more diagnostic and/or prognostic procedures). In some embodiments, system 10 is constructed and arranged to perform a lumen sizing procedure, such as a procedure in which one or more diameters of one or more lumen locations in the intestine are determined (e.g. estimated). In these embodiments, the relative location at which the diameter is determined can be maintained at a pressure at or near room pressure (e.g. via one or more lumens of catheter 100 and/or body introduction device 50). System 10 can be constructed and arranged to perform a patient imaging procedure, such as a procedure in which a patient image is collected, such as a patient image that includes functional assembly 130 positioned in a segment of the intestine. System 10 can be constructed and arranged to perform a tissue sampling procedure, such as in a biopsy procedure. In some embodiments, system 10 is constructed and arranged to perform a diagnostic and/or other procedure selected from the group consisting of: assessment of mucosal thickness and/or hypertrophy, such as while using OCT or similar imaging technologies; assessment of wall thickness, such as via endoscopic ultrasound or similar imaging technologies; visualization of enteroendocrine cell populations, such as via molecular imaging techniques or antibody labeling; assessment of the location of the ampulla of Vater, such as via bile acid labeling; and combinations of two or more of these. In some embodiments, system 10 is constructed and arranged to perform a therapeutic and/or other procedure selected from the group consisting of: an obesity treatment procedure, such as an endoluminal implant of a balloon or other volume reducing and/or restricting device in the stomach or small intestine, a suturing or anastomosing procedure to reduce and/or restrict gastrointestinal volume, and/or an intestinal bypass; a procedure including the injection of sclerosing material configured to induce scar formation; a procedure including the injection of material to create a therapeutic restriction; a procedure including the injection of drugs or other agents into the submucosal space; a microbial transplantation procedure, such as to alter gut microbial populations; and combinations of two or more of these.

In some embodiments, system 10 is constructed and arranged to perform a patient assessment, such as a patient screening to determine if an intestinal tissue ablation (e.g. a duodenal mucosa ablation) would benefit the patient. In these embodiments, system 10 and/or the methods of the present inventive concepts can be configured to compare glucagon administered orally (PO) versus glucagon administered intravenously (IV). Data gathered can include the difference in the patient's ability to suppress glucagon after a meal. Patient's whose ability to suppress glucagon falls below a threshold can be selected to receive a treatment of the present inventive concepts (e.g. an ablation or other treatment to at least the duodenal mucosa). Alternatively or additionally, analysis of fasting and/or postprandial glucagon can be compared to a threshold, and patients whose level is above the threshold can be selected to receive a treatment of the present inventive concepts (e.g. a treatment to at least the duodenal mucosa).

Catheter 100 of system 10 includes shaft 110, typically a flexible shaft comprising one or more lumens. In some embodiments, shaft 110 comprises varied flexibility along its length. In some embodiments, a bulbous tip is positioned on the distal end, tip 115, of catheter 100 as shown. Tip 115 can comprise a bulbous element with a diameter of at least 4 mm and/or a diameter less than or equal to 15 mm. In some embodiments, tip 115 comprises an inflatable bulbous tip. An operator graspable handle, handle 102 shown, is positioned on the proximal end of shaft 110. Handle 102 can comprise a user interface, such as user interface 105 shown. User interface 105 can comprise one or more user input components and/or user output components. User interface 105 can comprise one or more user input components configured to allow an operator to modify one or more operating parameters of console 200, settings 201, such as an operator-based modification based on information provided via a signal produced by a sensor of system 10. User interface 105 can comprise a control (e.g. control 104 described herebelow in reference to FIG. 2) or other user input component selected from the group consisting of: switch; keyboard; membrane keypad; knob; lever; touchscreen; and combinations of two or more of these. User interface 105 can comprise a user output component selected from the group consisting of: light such as an LED; display; touchscreen; audio transducer such as a buzzer or speaker; tactile transducer such as an eccentric rotational element; and combinations of two or more of these.

As described hereabove, functional assembly 130 can be constructed and arranged to perform a patient diagnosis and/or perform a patient treatment, such as a diagnosis and/or treatment performed on tissue of the intestine (e.g. mucosal and/or submucosal tissue of the intestine). In some embodiments, functional assembly 130 comprises an expandable assembly constructed and arranged to radially expand as determined by an operator of system 10. Functional assembly 130 can comprise an expandable element selected from the group consisting of: an inflatable balloon (e.g. balloon 136 as shown); a radially expandable cage or stent; one or more radially deployable arms; an expandable helix; an unfurlable compacted coiled structure; an unfurlable sheet; an unfoldable compacted structure; and combinations of two or more of these. Functional assembly 130 is shown in a radially expanded state in FIG. 1. Balloon 136 can comprise a compliant balloon, a non-compliant balloon and/or a balloon with compliant and non-compliant sections, as described hereabove. Balloon 136 can comprise a pressure-thresholded balloon, also as described hereabove. Balloon 136 can comprise a multi-layer construction, such as a construction with different materials positioned in different layers of balloon 136. In some embodiments, at least the distal portion of catheter 100, distal portion 100_{DP}, is constructed and arranged to be: inserted through an endoscope such as body introduction device 50; inserted alongside an endoscope; inserted over a guidewire such as guidewire 60; inserted through a sheath such as scope attachable sheath 80; and/or inserted through an introducer such as introducer 90 (e.g. an introducer sheath).

Positioned within shaft 110 are one or more conduits or lumens, conduits 111. Conduits 111 can comprise a conduit selected from the group consisting of: a fluid transport conduit (e.g. a tube or lumen configured to deliver fluids to functional assembly 130 and/or extract fluids from functional assembly 130); a tube comprising a lumen; a tube comprising a translatable rod; a hydraulic tube; a pneumatic tube; a tube configured to provide a vacuum (e.g. provide a vacuum to port 137 described herebelow); a lumen of shaft 110; an inflation lumen; a lumen configured to provide a vacuum (e.g. provide a vacuum to port 137); a fluid delivery lumen; a wire such as an electrically conductive wire; a linkage; a rod; a flexible filament; an optical fiber; and combinations of two or more of these. One or more conduits 111 can be configured to: transport fluid (e.g. deliver fluid and/or extract fluid); extract fluid; provide a positive pressure; provide a vacuum; and combinations of two or more of these. One or more conduits 111 can comprise a hollow tube, such as a tube comprising polyimide and/or a tube comprising a braid, such as a braided polyimide tube. One or more conduits 111 can be configured to allow the transport of: power, signals and/or materials such as fluids. A conduit 111 can be configured to slidingly receive a guidewire (e.g. guidewire 60), such as for over-the-wire delivery of catheter 100, such as when a conduit 111 is operably connected to a guidewire lumen, such as lumen 116 of tip 115. Alternatively, guidewire lumen 116 can both enter and exit distal portion 100_{DP} of catheter 100 (e.g. enter and exit tip 115 as shown in FIG. 1), such as for rapid-exchange manipulation of catheter 100 over a guidewire. In some embodiments, one or more conduits 111 can be translated within shaft 110 (e.g. advanced and/or retracted), such as to change the position of a distal end of a conduit 111 (e.g. to change the position of an outflow tube or inflow tube within functional assembly 130).

Shaft 110 can comprise one or more functional elements, such as functional element 119 shown. Functional element 119 can be positioned on (e.g. on the outer surface of), in (e.g. within the wall of) and/or within (e.g. within a lumen of) shaft 110. Functional element 119 can be positioned proximate (e.g. nearby, on, in and/or within) one or more conduits 111, such as when functional element 119 comprises a valve, heating element, and/or cooling element configured to exert a force and/or alter the temperature of one or more fluids passing within a conduit 111.

Functional assembly 130 can comprise one or more functional elements, element 139, such as treatment element 139a, sensor 139b and/or fluid delivery element 139c, all shown in FIG. 1. Each functional element 139 can comprise a sensor, a transducer and/or other functional element, as described in detail herein.

In some embodiments, one or more functional elements 139 are constructed and arranged as a tissue treatment element of the present inventive concepts, as described herein, such as when treatment element 139a comprises an energy delivery element configured to treat target tissue of the intestine. Treatment element 139a can be of similar construction and arrangement as treatment element 135 described herebelow in reference to FIG. 2. Treatment element 139a can comprise a treatment element selected from the group consisting of: an ablative fluid (e.g. an ablative fluid to be maintained within balloon 136 and/or an ablative fluid to be delivered onto tissue such as via a fluid delivery element 139c); an electrode configured to deliver radiofrequency (RF) or other electrical energy to tissue; an optical element (e.g. a lens or a prism) configured to deliver laser or other light energy to tissue; a sound energy delivery element such as a piezo crystal configured to deliver ultrasound or subsonic sound energy to tissue; an agent delivery element such as a needle, nozzle or other fluid delivery element configured to deliver an ablative or other agent onto and/or into tissue; and combinations of two or more of these. In some embodiments, treatment element 139a comprises fluid at an ablative temperature. In these embodiments, treatment element 139a can comprise fluid whose temperature changes, such as when system 10 is configured to introduce a fluid both at an ablative temperature (e.g. sufficiently hot or cold to ablate) and fluid at a neutralizing temperature (e.g. a cooling fluid or a warming fluid, respectively), such as when fluid at a neutralizing temperature is delivered within functional assembly 130 before and/or after fluid at an ablative temperature is delivered within functional assembly 130, as described in detail herein.

In some embodiments, treatment element 139a comprises an energy delivery element including multiple layers of electrical conductors (e.g. conductors and/or semiconductors) configured to generate heat when electricity passes through one or more of the conductors. In these embodiments, functional element 139 can be electrically connected to one or more conduits 111 comprising one or more electrical wires. Functional assembly 130 can comprise a compliant or non-compliant balloon onto which functional element 139 is positioned. Treatment element 139a can comprise electrical conductors created by depositing one or more coatings on one or more substrates. When electricity is passed through the coating, heat is generated. The heat can be effectively transferred across the whole surface of functional element 139 mainly through conduction, but also via radiation and convection and into target tissue.

In some embodiments, one or more functional elements 139 are constructed and arranged to perform a diagnosis and/or prognosis (“diagnosis” herein), such as when sensor 139b comprises a sensor configured to sense a physiologic parameter of intestinal tissue. Sensor 139b can comprise one or more sensors, such as are described in detail herebelow.

In some embodiments, one or more functional elements 139 are constructed and arranged to expand tissue, such as when fluid delivery element 139c comprises one or more of: a needle, nozzle, fluid jet, iontophoretic fluid delivery element, an opening in functional assembly 130 (e.g. an opening in balloon 136) and/or other fluid delivery element configured to deliver fluid into and/or onto tissue (e.g. into submucosal tissue). In some embodiments, fluid delivery element 139c comprises an element (e.g. a needle or fluid jet) configured to deliver fluid into tissue, such as submucosal tissue, to expand the tissue receiving the injected fluid. Alternatively or additionally, fluid delivery element 139c can comprise an element (e.g. a nozzle) configured to deliver fluid onto tissue, such as ablative fluid delivered onto tissue to ablate and/or remove tissue or neutralizing fluid configured to reduce tissue trauma (e.g. limit the volume of tissue ablated). Fluid delivery element 139c can comprise a needle selected from the group consisting of: a straight needle; a curved needle; a single lumen needle; a multiple lumen needle; and combinations of two or more of these. Fluid delivery element 139c can be positioned proximate and/or within a port, such as port 137 shown. Port 137 can be placed on top of balloon 136 and/or recessed into balloon 136 (e.g. positioned within a recess of balloon 136 or other component of functional assembly 130). Port 137 can be engaged between layers of balloon 136, such as when balloon 136 comprises multiple layers including an outer layer (e.g. a layer of PET material) that surrounds at least a portion of port 137. In some embodiments, port 137 comprises an insulating element, such as an insulating element configured to prevent full circumferential ablation of an axial segment of intestine. Alternatively, port 137 can be thermally conductive, to enhance heat or cryogenic ablation proximate port 137. Port 137 can be positioned on a tissue-contacting portion of balloon 136 as shown. Port 137 can be attached to a source of vacuum, such as vacuum provided by a conduit 111, such that port 137 can engage with the tissue. Port 137 can be constructed and arranged such that tissue can be drawn into an opening of port 137, such as when tissue is drawn into port 137 prior to delivery of fluid by fluid delivery element 139c into tissue, as described herein. In some embodiments, catheter 100 comprises multiple ports 137 and multiple corresponding fluid delivery elements 139c, such as two, three or more pairs of ports 137 and fluid delivery elements 139c (e.g. equally spaced about a circumference of balloon 136). One or more fluid delivery elements 139c can be attached to one or more conduits 111 and can be configured to be translated (e.g. translated within a tissue-capturing opening of port 137). Translation of a fluid delivery element 139c can be limited by one or more mechanical stops constructed and arranged to limit advancement and/or retraction of the fluid delivery element 139c. One or more fluid delivery elements 139c and a fluidly attached conduit 111 can be biased by one or more springs, such as one or more springs positioned in handle 102. Fluid delivery element 139c and an associated functional assembly 130 can be of similar construction and arrangement as those described herebelow in reference to catheter 20 and/or catheter 40 of FIG. 2, or as described in applicant's co-pending U.S. patent application Ser. No. 15/274,948 (Attorney Docket No. 41714-712.301; Client Docket No. MCT-027-US), entitled “Injectate Delivery Devices, Systems and Methods”, filed Sep. 23, 2016, the content of which is incorporated herein by reference in its entirety for all purposes. One or more fluid delivery element 139c can comprise a straight or a curved needle. One or more fluid delivery elements 139c can be constructed and arranged to enter tissue at an angle between 0° and 90°, such as at an angle between 30° and 60°.

In some embodiments, port 137 can be configured to engage tissue (e.g. when a vacuum is applied to port 137 via one or more conduits 111), after which target tissue can be treated by treatment element 139a. Engagement of tissue by port 137 can be used to stretch or otherwise manipulate tissue such that a safe and effective treatment of target tissue can be performed by treatment element 139a, such as when treatment element 139a comprises fluid at an ablative temperature or an array of electrodes configured to deliver RF energy. In these embodiments, catheter 100 can be configured to treat target tissue without performing an associated tissue expansion procedure (e.g. without expanding tissue in proximity to the target tissue to be treated).

Functional assembly 130 can be configured to treat target tissue, such as when functional element 139 comprises ablative fluid introduced into balloon 136 or when functional element 139 comprises one or more energy delivery elements as described herein. Functional assembly 130 can be constructed and arranged to treat a full or partial circumferential axial segment of intestinal tissue (e.g. intestinal mucosa). System 10 can be configured to treat multiple axial segments of tissue, such as multiple relatively contiguous or discontiguous segments of mucosal tissue treated simultaneously and/or sequentially. The multiple segments can comprise overlapping and/or non-overlapping borders.

Catheter 100 is configured to operably attach to console 200. In some embodiments, catheter 100 attaches directly to console 200. In other embodiments, attachment assembly 300 is positioned and operably attached between catheter 100 and console 200, such as to transfer materials (such as injectate 221, agent 420, hydraulic and/or pneumatic fluid, ablative fluids and/or other fluids), energy (such as ablative fluids and/or energy), and/or data between catheter 100 and console 200. Attachment assembly 300 comprises end 301 which attaches to catheter 100 via connector 103 of handle 102. Attachment assembly 300 further comprises end 302 which attaches to console 200 via connector 203 of console 200. Conduits 311 of attachment assembly 300 operably attach conduits 111 of catheter 100 to associated conduits 211 of console 200. Attachment assembly 300 can comprise a cassette configuration configured to operably attach to console 200. Attachment assembly 300 can comprise one or more flexible portions (e.g. coiled tubes and/or filaments) that allow movement of catheter 100 relative to console 200, such as to extend catheter 100 away from console 200 and toward a table onto which a patient is positioned. Attachment assembly 300 can comprise one or more functional elements, functional element 309 shown, such as an array of functional elements 309, each positioned proximate a conduit 311. Each functional element 309 can comprise a sensor, transducer and/or other functional element as described in detail herein.

Console 200 is configured to operably control and/or otherwise interface with catheter 100. In some embodiments, console 200 comprises one or more pumping assemblies, assembly 225 (four shown in FIG. 1), which can each be attached to a reservoir, reservoir 220 (four shown in FIG. 1) via one or more conduits 212. Each reservoir 220 can be constructed and arranged to store and supply fluids to catheter 100 and/or to extract fluids from catheter 100, such as is described herebelow in reference to system 10 of FIG. 2. An ablative fluid, a neutralizing fluid, agent 420 and/or injectate 221 can be placed or otherwise positioned within one or more reservoirs 220, such as to be transported by one or more pumping assemblies 225 into one or more conduits 111 of catheter 100 (e.g. via conduits 211 of console 200 and optionally via conduits 311 of connecting assembly 300). In some embodiments, console 200 is constructed and arranged to deliver a neutralizing fluid (e.g. a cooling fluid or warming fluid contained within a reservoir 220), then an ablative fluid (e.g. a hot fluid and/or a cryogenic fluid, respectively, contained within one or more reservoirs 220). In these embodiments, console 200 can be further constructed and arranged to subsequently deliver (i.e. after the ablation step), the same or a different neutralizing fluid (e.g. a cooling or warming fluid contained within a reservoir 200). In some embodiments, a first reservoir 220 provides an ablative fluid comprising a hot fluid at a temperature above 44° C., such as above 65° C., above 75° C., above 85° C. or above 95° C., and a second reservoir 220 provides a neutralizing fluid comprising a cooling fluid below 37° C., such as below 20° C. or below 15° C. In some embodiments, a first reservoir 220 provides an ablative fluid comprising a cryogenic fluid, and a second reservoir 220 provides a neutralizing fluid comprising a warming fluid at or above 37° C.

Alternatively or additionally, console 200 can be configured to provide RF and/or light energy to functional assembly 130 to ablate or otherwise treat tissue, and a cooling step can be performed (e.g. via a neutralizing fluid provided by a reservoir 220 comprising fluid below 37° C.) prior to and/or after the delivery of the RF and/or light energy. In some embodiments, system 10 comprises two return paths, one for recovery of ablative fluid (e.g. hot fluid), and one for recovery of neutralizing fluid (e.g. cooling fluid), such as via separate conduits 111, 311 and/or 211. In these embodiments, two separate pumping assemblies 225 can be fluidly attached to the separate return paths.

Console 200 comprises one or more console settings 201 that can be varied, such as a change made manually (e.g. by a clinician or other operator of system 10), and/or automatically by system 10. Console 200 can comprise a central processing unit, microcontroller, and/or other controller, controller 250 shown. Controller 250 can comprise one or more signal processors, such as signal processor 252 shown. Signal processor 252 can be configured to analyze one or more sensor signals, such as to modify one or more settings 201 of console 200. Controller 250 and/or signal processor 252 can comprise one or more algorithms, algorithm 251, which can be configured to perform one or more mathematical or other functions, such as to compare one or more sensor signals (e.g. compare the signal itself or a mathematical derivation of the signal) to a threshold. Console settings 201 can comprise one or more parameters (e.g. system parameters as also referred to herein) of catheter 100, console 200 and/or any component of system 10. Console settings 201 can comprise one or more parameters selected from the group consisting of: delivery rate of fluid into functional assembly 130; withdrawal rate of fluid from functional assembly 130; delivery rate of fluid into tissue; rate of energy delivered into tissue; peak energy level delivered into tissue; average energy delivery rate delivered into tissue; amount of energy delivered into tissue during a time period; temperature of an ablative fluid (e.g. temperature of an ablative fluid in reservoir 220, console 200, functional assembly 130 and/or catheter 100); temperature of a neutralizing fluid (e.g. temperature of a neutralizing fluid in reservoir 220, console 200, functional assembly 130 and/or catheter 100); temperature of functional assembly 130; pressure of functional assembly 130; pressure of fluid delivered into functional assembly 130; pressure of fluid delivered into tissue; duration of energy delivery; time of energy delivery (e.g. time of day of or relative time compared to another step); translation rate such as translation rate of a functional assembly 130; rotation rate such as rotation rate of a functional assembly 130; a flow rate; a recirculation rate; a heating rate or temperature; a cooling rate or temperature; a sampling rate (e.g. a sampling rate of a sensor); and combinations of two or more of these. In some embodiments, one or more console settings 201 comprise a setting related to a system 10 parameter selected from the group consisting of: pressure and/or volume of a fluid delivered to shaft 110 to change the stiffness of shaft 110 (e.g. to modify pushability and/or trackability of shaft 110); pressure and/or volume of a fluid delivered to and/or extracted from functional assembly 130 for inflation and/or deflation (e.g. to obtain apposition of ports 137 and/or to anchor functional assembly 130 in the intestine); pressure and/or volume of a fluid delivered to one or more conduits 111, each configured as a fluid transport tube to provide an injectate, injectate 221, to one or more fluid delivery elements 139c, such as to advance and/or retract one or more fluid delivery elements 139c and/or to deliver injectate 221 into tissue (e.g. submucosal tissue); pressure and/or volume of a fluid within one or more conduits 111, each configured to provide a vacuum to one or more ports 137 to engage the one or more ports 137 with tissue and/or to cause a fluid delivery element 139c to engage (e.g. penetrate) tissue; a force used to advance and/or retract one or more conduits 111 and/or one or more fluid delivery elements 139c; and combinations of two or more of these. In some embodiments, one or more console settings 201 comprise a setting related to a system 10 parameter selected from the group consisting of: temperature, flow rate, pressure and/or duration of fluid delivered to catheter 100 and/or functional assembly 130; temperature, flow rate, pressure and/or duration of fluid contained within functional assembly 130 and/or circulating loops (e.g. conduits 111, 211, and/or 311) of system 10; and combinations of two or more of these. System 10 can be configured to adjust one or more console settings 201 based on one or more signals produced by one or more sensors of system 10. Based on the one or more sensor signals, system 10 can be configured to modify a console setting 201 to cause: stopping delivery of fluid and/or energy to and/or by functional assembly 130; delivering additional fluid into functional assembly 130 and/or into tissue (e.g. adjust fluid delivery rate); delivering neutralizing and/or other additional fluid into functional assembly 130 and/or into tissue; adjusting the pressure of functional assembly 130; adjusting the volume of functional assembly 130; and combinations of two or more of these. In some embodiments, algorithm 251 is configured to determine an injectate delivery parameter, such as the amount (e.g. volume and/or mass) of injectate 221 to be delivered by catheter 100.

In some embodiments, system 10 adjusts, via algorithm 251, a functional assembly 130 parameter based on a signal of a sensor of system 10. In these embodiments, a functional assembly 130 parameter can be adjusted during performance of a procedural step, such as an ablation step and/or a tissue expansion step. The functional assembly 130 parameter adjusted can comprise a parameter selected from the group consisting of: volume of functional assembly 130; diameter of functional assembly 130; pressure of functional assembly 130; force applied to tissue by functional assembly 130; and combinations of two or more of these. The functional assembly 130 parameter can be adjusted to prevent excessive force being applied to the intestinal wall or to maintain a minimum apposition level of functional assembly 130 with tissue of the intestine.

In some embodiments, algorithm 251 is configured to determine an expanded size for functional assembly 130, such as when system 10 comprises multiple catheters 100 with different expanded diameters for functional assembly 130 and/or when the expanded diameter of functional assembly 130 can be varied by system 10 (e.g. by varying pressure and/or volume of fluid within functional assembly 130). In these embodiments, algorithm 251 can comprise a bias, such as a bias which tends toward lower diameters (e.g. rounds down to the next smaller size of a functional assembly 130 available after calculating a target value). In some embodiments, algorithm 251 is configured to select one catheter 100 for use in a patient, by selecting one from a kit of multiple catheters 100 comprising one or more different parameters (e.g. one or more functional assembly 130 parameters). In these embodiments, algorithm 251 can also include a bias, such as a bias toward choosing a smaller functional assembly 130 (e.g. smaller length or smaller expanded diameter).

In some embodiments, algorithm 251 comprises an image analysis algorithm configured to analyze one or more patient and/or system 10 images. For example, a tissue location can be analyzed prior to, during and/or after a desufflation (e.g. aspiration) step, such as to confirm adequate apposition of a functional assembly 130 with tissue of an axial segment of tubular tissue (e.g. an axial segment of the intestine). Algorithm 251 can comprise one or more image analysis algorithms configured to assess various conditions including but not limited to: apposition of functional assembly 130 with tissue (e.g. intestinal wall tissue); effectiveness of a desufflation procedure; effectiveness of an insufflation procedure; sufficiency of a tissue expansion procedure; sufficiency of a tissue ablation procedure; and combinations of two or more of these.

In some embodiments, algorithm 251 can be configured to operatively adjust one or more operating parameters (generally console settings 201) of console 200 and/or catheter 100, such as an algorithm 251 that analyzes data provided by one or more sensors of system 10. Algorithm 251 can be configured to correlate a signal received by one or more sensors of system 10 positioned at a first location, to a parameter of system 10 or the patient at a second location distant from the first location (e.g. a second location proximal or distal to the first location). For example, a measured temperature or pressure within console 200 (e.g. via functional element 229a or 229b), connecting assembly 300 (e.g. via functional element 309), and/or catheter 100 (e.g. via functional element 119), can provide a signal related to a parameter at a remote location, such as a parameter of functional assembly 130 or the patient (e.g. a physiologic parameter at a location within the patient). Algorithm 251 can be configured to analyze a signal received from a first location and produce parameter information correlating to a second location.

In some embodiments, algorithm 251 comprises a pressure algorithm configured to modify a system parameter based on a measured pressure, such as a modification made based on the pressure within a luminal segment of the intestine in which functional assembly 130 is positioned or otherwise proximate (e.g. as measured or otherwise determined by analysis of a signal provided by a sensor of catheter 100, body introduction device 50 or another sensor of system 10 as described herein). In these embodiments, system 10 can be configured to modify the volume of fluid within functional assembly 130 and/or modify the pressure of functional assembly 130 based on the luminal segment pressure.

In some embodiments, system 10 is constructed and arranged to produce an image (e.g. an image produced by an imaging device and/or other sensor of the present inventive concepts). Algorithm 251 can be configured to analyze one or more images of tissue that are visualized through one or more portions of functional assembly 130, such as to determine the level of tissue expansion and/or a level of tissue ablation, such as to assess completion adequacy of one or more steps of a medical procedure.

In some embodiments, console 200 comprises a first reservoir 220 containing hot fluid for ablation, a second reservoir 220 comprising cooling fluid at a first temperature (e.g. a temperature less than 37° C. but more than 10° C.), and a third reservoir 220 comprising fluid at a second temperature cooler than the first temperature (e.g. a temperature less than 6° C., such as a temperature between 2° C. and 4° C.). Fluid from the third reservoir 220 can be delivered into the second reservoir 220 (e.g. after one or more steps including cooling and ablation of tissue have been performed).

In some embodiments, console 200 comprises a first reservoir 220 containing hot fluid for ablation at a first temperature (e.g. approximately 55° C.), and a second reservoir 220 comprising hot fluid for ablation at a second temperature (e.g. approximately 95° C.). Fluid from the first reservoir 220 and the second reservoir 220 can be delivered to functional assembly 130 for equal time periods. In these embodiments, console 200 can further comprise a third reservoir 220 comprising cooling fluid, such as when console 200 is configured to deliver hot fluid from the first reservoir 220, followed by hot fluid from the second reservoir 220, followed by cooling fluid from the third reservoir 220. Console 200 can be further configured to deliver the cooling fluid prior to the delivery of the hot fluid from the first reservoir 220. In some embodiments, fluid from a reservoir 220 is delivered for a time period determined based on the temperature of fluid in that reservoir and/or based on the temperature of fluid in a separate reservoir 220, as described herebelow. For example, the amount of ablative fluid delivered by a reservoir 220 containing hot fluid can be adjusted based on the temperature of cooling fluid in a different reservoir 220.

In some embodiments, console 200 comprises multiple functional elements 209 (four shown in FIG. 1), such as a first functional element 209 comprising a heating element and a second functional element 209 comprising a cooling element. In these embodiments, connecting assembly 300 can comprise a tubeset configured to be engaged with console 200 to allow the first functional element 209 to transfer heat into fluid within connecting assembly 300 and the second functional element 209 to extract heat from (i.e. cool) fluid within connecting assembly 300. In these embodiments, system 10 can avoid the need for heated and/or cooled reservoirs 220, such as when console 200 further comprises a disposable fluid supply fluidly attached to connecting assembly 300. Connecting assembly 300 can comprise a reusable tubing set. Connecting assembly 300 can comprise a tubing set comprising multiple lumens (e.g. multiple tubes each with one or more lumens, or a single tube with multiple lumens), such as at least a first lumen configured to deliver inflation fluid (e.g. deliver inflation fluid to functional assembly 130 to perform a tissue expansion procedure and/or a tissue sizing procedure), and at least two lumens configured to deliver a recirculating fluid (e.g. to recirculate ablative fluid and/or neutralizing fluid within functional assembly 130 during a tissue ablation procedure).

Console 200 can comprise a user interface, user interface 205 shown, which can deliver commands to controller 250 and receive information (e.g. to be displayed) from controller 250. In some embodiments, console 200 comprises an energy delivery unit, EDU 260, such as an energy delivery unit configured to provide one or more of: thermal energy such as heat energy or cryogenic energy; electromagnetic energy such as radiofrequency (RF) energy; light energy such as light energy provided by a laser; sound energy such as subsonic energy or ultrasonic energy; chemical energy (e.g. a chemically ablative substance); and combinations of two or more of these. EDU 260 can be of similar construction and arrangement as EDU 260 described herebelow in reference to FIG. 2. Console 200 can further comprise conduits 211 which can be operably connected to catheter 100 (e.g. operably connected to one or more conduits 111 or other components of catheter 100). Conduits 211 can comprise one or more fluid transport tubes fluidly attached to pumping assemblies 225 and/or any filament bundle operably attached to controller 250 and comprising one or more filaments selected from the group consisting of: a tube comprising a lumen; a tube comprising a translatable rod; a hydraulic tube; a pneumatic tube; a tube configured to provide a vacuum (e.g. provide a vacuum to port 137); a lumen of shaft 110; an inflation lumen; a fluid delivery lumen; a wire such as an electrically conductive wire; a linkage; a rod; a flexible filament; an optical fiber; and combinations of two or more of these. Controller 250 can be operably connected to one or more of reservoirs 220, pumping assemblies 225 and/or user interface 205 via a bus, bus 213 shown. Bus 213 can comprise one or more wires, optical fibers, and/or other conduits configured to provide power, transmit data and/or receive data.

In some embodiments, console 200 is configured to operably expand functional assembly 130, such as with a liquid, gas, and/or other fluid provided by a reservoir 220 and propelled by an associated pumping assembly 225. In some embodiments, console 200 is configured to deliver fluid to tissue via one or more fluid delivery elements 139c, such as with a fluid (e.g. injectate 221) provided by a reservoir 220 and propelled by an associated pumping assembly 225. In some embodiments, console 200 is configured to deliver ablative fluid to functional assembly 130, such as ablative fluid provided by a reservoir 220 and propelled by an associated pumping assembly 225. In these embodiments, ablative fluid can be recirculated to and from functional assembly 130 by console 200. In some embodiments, console 200 is configured to deliver energy, such as electromagnetic or other energy, to functional assembly 130, such as via controller 250. Each of these embodiments is described in detail herebelow in reference to system 10 of FIG. 2.

One or more reservoirs 220 can each comprise one more functional elements 229a and/or one or more pumping assemblies 225 can each comprise one or functional elements 229b. Each functional element 229a and/or 229b (singly or collectively functional element 229) can comprise a sensor, a transducer, and/or other functional element. In some embodiments, one or more functional elements 229 comprise a heating element or a chilling element configured to heat or chill fluid within a reservoir 220 and/or a pumping assembly 225. Alternatively or additionally, one or more functional elements 229 comprise a sensor, such as a temperature sensor, pressure sensor and/or a flow rate sensor configured to measure the temperature, pressure and/or flow rate, respectively, of fluid within, flowing into, and/or flowing out of a reservoir 220 and/or pumping assembly 225.

In some embodiments, console 200 operably attaches to and controls multiple catheters 100, such as two or more catheters 100 of similar construction and arrangement to catheters 100, 20, 30 and/or 40 described herebelow in reference to FIG. 2.

In some embodiments, system 10 comprises a first catheter 100 with a functional assembly with a first diameter, and a second catheter 100 with a functional assembly with a second diameter (e.g. a smaller expanded diameter than the first diameter). In these embodiments, system 10 can be constructed and arranged such that an operator (e.g. a clinician) inserts the first catheter 100 into the intestine of a patient and performs a first function, such as a function selected from the group consisting of: size (e.g. determine the diameter) of one or more axial locations of intestine; perform or at least attempt to perform a tissue expansion procedure in one or more axial segments of intestine; perform or at least attempt to perform a tissue treatment (e.g. tissue ablation) at one or more axial segments of intestine; and combinations of two or more of these. In some embodiments, during and/or after performance of the first function, a decision can be made to switch to the second catheter 100 with a different functional assembly 130, such as when it is determined the functional assembly 130 of the first catheter 100 is too large. In these embodiments, the first catheter 100 and the second catheter 100 can each be configured to perform both a tissue expansion procedure and an ablation procedure. In some embodiments, the functional assembly 130 of the first catheter 100 comprises an expanded diameter between 21 mm and 29 mm, such as a diameter between 23 mm and 27 mm, such as a diameter of approximately 25 mm. In some embodiments, algorithm 251 is configured to select the first catheter 100 and/or the second catheter 100 for use (e.g. use in the patient). Alternatively, the functional assembly 130 of the first catheter 100 can comprise an expanded diameter smaller than the expanded diameter of the functional assembly 130 of the second catheter 100, wherein the second catheter 100 is introduced into the patient if it is determined that the expanded diameter of the functional assembly 130 of the first catheter 100 is too small.

In some embodiments, system 10 comprises one or more first catheters 100a, each with a first functional assembly 130a of a particular size and configured to treat target tissue. System 10 further comprises one or more second catheters 100b, each with a functional assembly 130b and configured to perform a tissue expansion procedure. System 10 can be constructed and arranged to size a lumen of one or more axial segments of intestine (e.g. using a catheter 100 or other luminal sizing device as described herein) to determine the diameter at a relatively narrow (e.g. the smallest diameter) location within the one or more axial segments to be treated. System 10 is further constructed and arranged to select a first catheter 100a based on the luminal sizing information (e.g. using algorithm 251). System 10 can be constructed and arranged to inflate or otherwise expand the functional assembly 130a of a catheter 100a (e.g. with an ablative fluid) to a diameter related to the smallest diameter location. System 10 can be constructed and arranged to inflate or otherwise expand the functional assembly 130b of a catheter 100b (e.g. with a gas) to a diameter corresponding to the expanded diameter of the selected catheter 100a (e.g. a diameter less than the proximate axial segment lumen size and/or to a diameter related to the smallest diameter location). System 10 can be constructed and arranged to apply a vacuum to one or more ports 137 of catheter 100b to engage neighboring tissue. System 10 can be further constructed and arranged to inject fluid into tissue (e.g. submucosal tissue) until the pressure within the associated functional assembly 130b exceeds a threshold, such as a threshold of 0.3 psi, 0.5 psi or 0.7 psi (e.g. but below a second threshold of 2.0 psi or 4.0 psi). System 10 can be constructed and arranged to subsequently disengage functional assembly 130b from the tissue (e.g. by removal of the vacuum from each port 137), and radially collapse balloon 136 (e.g. via extraction of fluid from balloon 136 via one or more conduits 111 of catheter 100b). System 10 can be constructed and arranged to similarly expand tissue at one or more other axial segments of the intestine. System 10 can be constructed and arranged to treat target tissue of the one or more axial segments (with expanded tissue) using the particular first catheter 100a whose expanded diameter was chosen based on the minimum diameter of the one or more axial segments. In some embodiments, multiple tissue expansion procedures are performed by catheter 100b sequentially, after which a series of target tissue treatments (e.g. tissue ablations) are performed by catheter 100a sequentially. Alternatively, a pattern of alternating between one or more tissue expansions and one or more tissue treatments can be performed.

In some embodiments, system 10 comprises one or more first catheters 100a, each with a first functional assembly 130a of a particular size and configured to treat target tissue. System 10 can further comprise one or more second catheters 100b, each with a functional assembly 130b and configured to perform a tissue expansion procedure. System 10 can be constructed and arranged to size a lumen of one or more axial segments of intestine (e.g. using a catheter 100 or other luminal sizing device as described herein) to determine the diameter at a relatively narrow (e.g. the smallest diameter) location within the one or more axial segments. System 10 can be further constructed and arranged to select a first catheter 100a based on the luminal sizing information (e.g. using algorithm 251). System 10 is further constructed and arranged to inflate the functional assembly 130b of a catheter 100b (e.g. with a gas) to a pressure sufficient to correlate to sufficient apposition with the axial segment luminal wall. System 10 can be further constructed and arranged to apply a vacuum to one or more ports 137 of catheter 100b to engage neighboring tissue. System 10 can be further constructed and arranged to inject fluid into tissue (e.g. submucosal tissue) until the pressure within the associated functional assembly 130b exceeds a threshold, at which time fluid (e.g. air) can be extracted from functional assembly 130b and fluid delivery by fluid delivery element 139c continues until the volume of functional assembly 130b reaches a pre-determined lower limit.

As described hereabove, system 10 can include injectate 221. In some embodiments, injectate 221 comprises a material selected from the group consisting of: water; saline; a gel; a hydrogel; a protein hydrogel; a cross-linked hydrogel; a cross-linked polyalkyleneimine hydrogel; a dissolvable hydrogel (such as a supramolecular self-assembly hydrogel, a thiol-thioester exchange hydrogels and/or thiol-disulfide exchange hydrogels), autologous fat; collagen; bovine collagen; human cadaveric dermis; hyaluronic acid; calcium hydroxylapatite; polylactic acid; semi-permanent PMMA; dermal filler; gelatin; mesna (sodium 2-sulfanylethanesulfonate); and combinations of two or more of these. In some embodiments, injectate 221 comprises beads (e.g. pyrolytic carbon-coated beads) suspended in a carrier (e.g. a water-based carrier gel). In some embodiments, injectate 221 comprises a solid silicone elastomer (e.g. heat-vulcanized poly dimethylsiloxane) suspended in a carrier, such as a bio-excretable polyvinylpyrrolidone (PVP) carrier gel. In some embodiments, injectate 221 has an adjustable degradation rate, such as an injectate 221 comprising one or more cross linkers in combination with polyalkylene imines at specific concentrations that result in hydrogels with adjustable degradation properties. In some embodiments, injectate 221 and/or agent 420 comprises living cells, such as living cells injected into the mucosa or submucosa of the intestine to provide a therapeutic benefit.

In some embodiments, injectate 221 comprises a visualizable and/or otherwise detectable (e.g. magnetic) material (e.g. in addition to one or more materials of above) selected from the group consisting of: a dye; a visible dye; indigo carmine; methylene blue; India ink; SPOT™ dye; a visualizable media; radiopaque material; radiopaque powder; tantalum; tantalum powder; ultrasonically reflective material; magnetic material; ferrous material; and combinations of two or more of these.

In some embodiments, injectate 221 comprises a material selected from the group consisting of: a peptide polymer (e.g. a peptide polymer configured to stimulate fibroblasts to produce collagen); polylactic acid; polymethylmethacrylate (PMMA); a hydrogel; ethylene vinyl alcohol (EVOH); a material configured to polymerize EVOH; dimethyl sulfoxide (DMSO); saline; material harvested from a mammalian body; autologous material; fat cells; collagen; autologous collagen; bovine collagen; porcine collagen; bioengineered human collagen; dermis; a dermal filler; hyaluronic acid; conjugated hyaluronic acid; calcium hydroxylapatite; fibroblasts; a sclerosant; an adhesive; cyanoacrylate; a pharmaceutical agent; a visualizable material; a radiopaque material; a visible dye; ultrasonically reflective material; and combinations of two or more of these. As described herein, in some embodiments a volume of injectate 221 is delivered into tissue to create a therapeutic restriction (e.g. a therapeutic restriction with an axial length between 1 mm and 20 mm), as described herein, or as is described in applicant's co-pending U.S. patent application Ser. No. 16/267,771 (Attorney Docket No. 41714-711.302; Client Docket No. MCT-024-US-CON1) entitled “Systems, Devices and Methods for the Creation of a Therapeutic Restriction in the Gastrointestinal Tract”, filed Feb. 5, 2019, the content of which is incorporated herein by reference in its entirety for all purposes. In some embodiments, a volume of injectate 221 is delivered into tissue to create a safety margin of tissue that is created (e.g. an expanded tissue layer is created) prior to an ablation procedure, as is described herein.

In some embodiments, injectate 221 comprises a fluorescent-labeled material or other biomarker configured to identify the presence of a biological substance, such as to identify diseased tissue and/or other tissue for treatment by functional assembly 130 (e.g. to identify target tissue). For example, injectate 221 can comprise a material configured to be identified by imaging device 55 (e.g. identify a visualizable change to injectate 221 that occurs after contacting one or more biological substances). In these embodiments, imaging device 55 can comprise a molecular imaging device, such as when imaging device 55 comprises a molecular imaging probe and injectate 221 comprises an associated molecular imaging contrast agent. In these embodiments, injectate 221 can be configured to identify diseased tissue and/or to identify a particular level of one or more of pH, tissue oxygenation, blood flow, and the like. Injectate 221 can be configured to be delivered onto the surface of an intestinal lumen or other luminal surface tissue, and/or to be delivered into tissue (i.e. beneath the surface).

In some embodiments, injectate 221 comprises a material selected from the group consisting of: autologous fat; collagen; bovine collagen; human cadaveric dermis; hyaluronic acid; calcium hydroxylapatite; polylactic acid; semi-permanent PMMA; dermal filler; gelatin; and combinations of two or more of these. In some embodiments, injectate 221 comprises a material whose viscosity changes (e.g. increases) after delivery into tissue, such as a fluid whose viscosity increases as it is heated to body temperature.

In some embodiments, injectate 221 comprises a material including hollow materials and a carrier material, such as when system 10 is constructed and arranged to deliver injectate 221 to create a therapeutic restriction. In these embodiments, injectate 221 can comprise a material as described in US Patent Application US20080107744 or US Patent Application US20110091564, the contents of each of which is incorporated herein by reference in its entirety for all purposes. In some embodiments, injectate 221 comprises inorganic fibers and a carrier material. The inorganic fibers can be constructed and arranged to prevent or otherwise reduce their migration within tissue. The carrier material can be constructed and arranged to allow the inorganic fibers to be injectable (e.g. to pass through fluid delivery element 139c). In these embodiments, injectate 221 can comprise a material as described in US Patent Application US20140255458, the contents of which is incorporated herein by reference in its entirety for all purposes.

In some embodiments, system 10, console 200 and/or catheter 100 is constructed and arranged to reduce risk during injection of material (e.g. injectate 221) into the wall of the duodenum. In some embodiments, a pre-determined volume of polymer or other material is injected using catheter 100 or a standard endoscopic needle device. A volume of at least 0.5 ml, 1 ml, or 2.5 ml of a first material (e.g. a relatively inert material such as sterile saline), is injected into the wall first, creating a first expanded tissue volume, a “bleb” of expanded tissue and the saline. Subsequently, a second material, such as a pharmaceutical agent, a durable material (e.g. to create a therapeutic restriction as described herein), or other active material is injected into the first expanded tissue volume to further expand the tissue. In some embodiments, a visualization step is performed prior to injection of the second material, such as to confirm the location of the injected first material, and/or to confirm the location of the second material injection site (e.g. a visualization step used as a confirmation of proper location of injection).

In some embodiments, injectate 221 comprises a system 10 or operator detectable material such as a visualizable material, magnetic material or other detectable material. In some embodiments, injectate 221 comprises one or more materials (e.g. hyaluronic acid, a hydrogel, and/or a biocompatible polymer or copolymer such as ethylene vinyl alcohol), and can further include a detectable material selected from the group consisting of: a radiopaque material; barium sulfate; tantalum; ultrasonically reflecting material; magnetic material; a visible dye; and combinations of two or more of these. In these embodiments, system 10 can comprise a fluid extraction assembly comprising one or more ports 137 that are constructed and arranged to withdraw fluids from within the intestine, such as via one or more conduits 111 and one or more pumping assemblies 225. One or more functional elements 109, 119, 139, 229 and/or 309 can comprise a sensor configured to produce a signal related to the quantity of injectate 221 recovered via the one or more ports 137, such as a sensor configured to detect a volume, mass, flow rate and/or other parameter of injectate 221. Signal processor 252 can be configured to assess tissue expansion based on an analysis of the recovered injectate 221.

In some embodiments, injectate 221 comprises one or more materials such as ethylene vinyl alcohol (EVOH) which is provided in a liquid solvent such as dimethyl sulfoxide (DMSO). In these embodiments, a visualizable material such as a radiopaque material (e.g. tantalum) can be further included. In these embodiments, catheter 100 can be configured to deliver this injectate 221 into tissue (e.g. via one or more fluid delivery elements 139c), after which the one or more materials, and the visualizable material if included, precipitate from the solution to form a spongy implant, which can remain in proximity to the injection site for a prolonged period of time.

In some embodiments, functional assembly 130 is anchored to the intestine (e.g. by expanding functional assembly 130 and/or by having port 137 engage tissue). In these embodiments, a tissue expansion procedure can be performed, such as by advancing at least one fluid delivery element 139c (e.g. at least three fluid delivery elements 139c) into tissue and delivering injectate 221 (e.g. into submucosal tissue). Alternatively or additionally, a tissue ablation procedure can be performed.

In some embodiments, system 10 is configured to deliver injectate 221 into tissue, such as via one or more fluid delivery elements 139c, each of which can be positioned in a port 137. The delivery of injectate 221 into tissue can produce a therapeutic restriction, occlude one or more body conduits (e.g. blood vessels), deliver a (single) bolus of drug or other agent into blood or other tissue, create a drug or other agent “depot” in tissue, and combinations of two or more of these. Injectate 221 can be configured to expand after delivery into tissue. Injectate 221 can be configured to remain relatively “in place” within tissue proximate the injection site for at least one month, three months, six months, or one year. Injectate 221 can be delivered into tissue (e.g. via fluid delivery element 139c) in a location selected from the group consisting of: lower stomach; pylorus; proximal small intestine; distal small intestine; duodenum; jejunum; terminal ileum; bowel; and combinations of two or more of these. In some embodiments, injectate 221 comprises a hydrogel, such as to create a hydrogel prosthesis within one or more tissue layers of the intestine (e.g. one or more submucosal tissue layers).

Injectate 221 can be delivered into tissue to create a therapeutic restriction, as described herein, such as to create a space occupying obstruction as a treatment for obesity, Type 2 diabetes; hypercholesterolemia, hypertension; non-alcoholic fatty liver disease; non-alcoholic steatohepatitis; and/or other metabolic disease. Injectate 221 can be delivered to one or more tissue locations to create a sense of satiety, reduce chime throughput and/or reduce obesity. Injectate 221 can be injected into the gastric varices, such as when injectate 221 comprises an occlusive agent such as an adhesive such as cyanoacrylate. System 10 can be constructed and arranged such that delivery of injectate 221 into one or more tissue locations alters nutrient absorption and/or hormonal signaling from the mucosa. System 10 can be constructed and arranged to deliver injectate 221 into colon tissue (e.g. to expand colon submucosal tissue), such as to treat fecal incontinence.

As described above, system 10 can be constructed and arranged to deliver injectate 221 into tissue to deliver a bolus of medication and/or to create a drug or other agent depot within tissue of the patient, such as within mucosal tissue and/or submucosal tissue of the intestine. In some embodiments, an injectate 221 positioned within tissue is activated based on one or more signals produced by a sensor, such as a bioactive glucose sensor that responds to the detection of an analyte and leads to (e.g. via one or more components of system 10) release or other activation of injectate 221. For example, injectate 221 can comprise an anti-diabetic agent, such as insulin, and a sensor (e.g. implant 192 configured as a sensor) can comprise a glucose sensor that detects a glucose change, such as the higher glucose levels that occur after a meal. Injectate 221 can comprise a drug or other agent selected from the group consisting of: a steroid; an anti-inflammatory agent; a chemotherapeutic; a proton pump inhibitor; a sclerosant agent; a differentiation factor such as trans-retinoic acid; an anti-hyperglycemic agent such as GLP-1 analogue or others; an anti-obesity agent; an anti-hypertensive agent; an anti-cholesterol agent such as a statin or others; and combinations of two or more of these. In some embodiments, injectate 221 comprises a steroid or other anti-inflammatory agent delivered to a therapeutic restriction of the present inventive concepts (e.g. delivered into an existing restriction or to create a restriction). In some embodiments, injectate 221 comprises one or more steroids and/or other anti-inflammatory agents delivered to the site of chronic inflammation, such as a site of ulcerative colitis or Crohn's disease. In some embodiments, injectate 221 comprises one or more steroids or other anti-inflammatory agents delivered at the site of celiac disease (e.g. the proximal small intestine) and/or otherwise delivered to treat celiac disease. In some embodiments, injectate 221 comprises one or more chemotherapeutic agents delivered to the site of a cancerous or pre-cancerous lesion.

Injectate 221 can be injected into tissue in a single procedure or multiple procedures. System 10 can be configured to determine an injectate 221 delivery parameter (e.g. determined by algorithm 251), such as by performing an analysis based on a patient demographic parameter and/or a patient physiologic parameter, such as age, weight, HbA1c level and cholesterol level. The injectate delivery parameter can comprise a parameter selected from the group consisting of: volume of injectate 221 delivered; length and/or area of a tissue layer receiving injectate 221; type of material included in injectate 221; viscosity of injectate 221; titration result of injectate 221; and combinations of two or more of these.

In some embodiments, system 10 is constructed and arranged to both deliver a durable injectate 221 (e.g. injectate 221 remains in place for at least one month), as well as treat target tissue (e.g. a treatment comprising ablating duodenal and/or other intestinal mucosa). The two procedures can be performed on the same day or on different days.

In some embodiments, injectate 221 comprises a radiographic material, such as tantalum, such as to be used in combination with X-ray or fluoroscopy to assess tissue expansion (e.g. submucosal tissue expansion), as described herein. Alternatively or additionally, injectate 221 can comprise a material that is visualizable under other imaging modalities (e.g. an imaging modality provided by imaging device 55), such as magnetic material; ferrous material; ultrasonically reflective material; and combinations of two or more of these.

In some embodiments, sensor 139b comprises a sensor configured to provide an impedance measurement, such as an impedance measurement used by algorithm 251 to enable closed-loop or otherwise adjust delivery of RF energy from treatment element 139a. In some embodiments, injectate 221 comprises a conductive substance, such as a conductive substance configured to enhance an impedance measurement recorded by sensor 139b. In these embodiments, injectate 221 can comprise one or more substances that are both conductive and visualizable (e.g. visualizable by imaging device 55 as described hereabove), such as tantalum.

In some embodiments, injectate 221 comprises a pharmaceutical drug or other agent (e.g. injectate 221 comprises agent 420) configured to provide a therapeutic benefit when delivered by one or more fluid delivery elements 139c into intestinal or other tissue. In these embodiments, injectate 221 can be injected into mucosal tissue and/or tissue proximate mucosal tissue (e.g. submucosal tissue). A major function of the mucosa is to bind or absorb certain molecules, and the mucosa can prevent or otherwise reduce the passage of all other molecules. Thus, insertion of injectate 221 directly into the submucosa can bypass the mucosal barrier, enabling the delivery of therapeutic large molecules that otherwise would be passed through the body completely or largely unabsorbed. This procedure also provides more precise dosage control, since the amount of absorption through the mucosa can be variable. Injectate 221 (e.g. injectate 221 comprising agent 420) can comprise any therapeutic biologic or biochemical entity. The entity of injectate 221 can have therapeutic effect by itself or it can be externally triggered, such as when injectate 221 comprises trigger materials, such as magnetic nanoparticles triggered by magnetic fields, gold nanoparticles triggered by light, optical or other fields, particles activated by light such as ultraviolet light or infrared light, and/or particles activated by heating or chilling. In some embodiments, tool 500 is configured to provide the triggering event, such as by generating a magnetic field, delivering light, and/or by delivering or extracting heat.

Local administration of drugs with high systemic toxicity and/or propensity for resistance by catheter 100 is advantageous, as much higher local concentrations of the drug and/or much lower systemic bioavailability can be achieved. Avoidance of skin-penetrating injections can be beneficial (e.g. avoiding associated pain, cosmetic issues and likely trauma to injection site). Catheter 100 can be used to deliver depot formulations of drugs or other agents to intestinal tissue (e.g. the intestinal submucosa) for the treatment of various GI or systemic illnesses. Submucosal delivery via catheter 100 can avoid the limitations associated with the mucosal barrier as described hereabove, and the limited bioavailability that is created. Submucosal delivery via catheter 100 can also allow the delivered drug to avoid chemical reactions or other adverse effects that result from interaction with various microbiological and pH environments in the patient's gut. In some embodiments, injectate 221 comprises an anti-reflux medication and/or an anti-acid medication, such as when injectate 221 is delivered into the mucosa or submucosa of the esophagus, intestine and/or stomach.

Systemic pharmaceutical therapy including immunomodulators to treat inflammatory bowel disease has issues with toxicity associated with immune suppression. This systemic therapy can also have limited efficacy once an individual develops antibodies against the monoclonal antibody therapies. In both cases, high systemic concentrations of the drugs limit the ability to achieve sufficiently effective doses in the GI tract itself, where the therapy needs to be most effective. A catheter 100 comprising one or more fluid delivery elements 139c can be used as a tool to perform site-specific delivery of drugs and other agents to treat GI illnesses, such as celiac disease and inflammatory bowel disease.

In some embodiments, injectate 221 comprises an injectate configured to cause inflammation of tissue. In these embodiments, one or more fluid delivery elements 139c can be configured to deliver the injectate 221 to tissue to cause an inflammatory response in the tissue. The inflammatory response can result in a tissue layer that functions as a protective layer during a subsequent tissue treatment procedure (e.g. tissue ablation procedure) performed by a functional assembly 130 of catheter 100.

As described hereabove, system 10 can include agent 420, which can include one or more agents delivered to the patient (e.g. orally, transdermally, via injection, or otherwise). In some embodiments, agent 420 comprises a material selected from the group consisting of: anti-peristaltic agent, such as L-menthol (i.e. oil of peppermint); glucagon; buscopan; hycosine; somatostatin; a diabetic medication; an analgesic agent; an opioid agent; a chemotherapeutic agent; a hormone; and combinations of two or more of these.

In some embodiments, agent 420 comprises cells delivered into the intestine, such as living cells delivered into intestinal mucosa or submucosa, such as via a fluid delivery element 139c or otherwise.

As described hereabove, system 10 can comprise one or more sensors, transducers and/or other functional elements, such as functional element 109, functional element 119 and/or functional element 139 (e.g. 139a, 139b and/or 139c) of catheter 100 and/or functional element 209 and/or functional element 229 (e.g. 229a and/or 229b) of console 200. In some embodiments, system 10 comprises connecting assembly 300 which can include one or more functional elements 309.

In some embodiments, one or more functional elements 109, 119, 139, 209, 229 and/or 309 comprise a transducer selected from the group consisting of: an energy converting transducer; a heating element; a cooling element such as a Peltier cooling element; a drug delivery element such as an iontophoretic drug delivery element; a magnetic transducer; a magnetic field generator; a sound generator; an ultrasound wave generator such as a piezo crystal; a light producing element such as a visible and/or infrared light emitting diode; a motor; a pressure transducer; a vibrational transducer; a solenoid; a fluid agitating element; and combinations of two or more of these.

In some embodiments, one or more functional elements 109, 119, 139, 209, 229 and/or 309 comprise a visualizable element, such as an element selected from the group consisting of: a radiopaque marker; an ultrasonically visible marker; an infrared marker; a marker visualizable by a camera such as an endoscopic camera; a marker visualizable by an MRI; a chemical marker; and combinations of two or more of these.

In some embodiments, one or more of functional elements 109, 119, 139, 209, 229 and/or 309 comprise a sensor configured to produce a signal, the sensor selected from the group consisting of: physiologic sensor; blood glucose sensor; blood gas sensor; blood sensor; respiration sensor; EKG sensor; EEG sensor; neuronal activity sensor; blood pressure sensor; flow sensor such as a flow rate sensor; volume sensor (e.g. a volume sensor used to detect a volume of injectate 221 not delivered into tissue); pressure sensor; force sensor; sound sensor such as an ultrasound sensor; electromagnetic sensor such as an electromagnetic field sensor or an electrode; gas bubble detector such as an ultrasonic gas bubble detector; strain gauge; magnetic sensor; ultrasonic sensor; optical sensor such as a light sensor; chemical sensor; visual sensor such as a camera; temperature sensor such as a thermocouple, thermistor, resistance temperature detector or optical temperature sensor; impedance sensor such as a tissue impedance sensor; and combinations of two or more of these. Each sensor can be configured to produce a signal that directly correlates to or is otherwise related to a patient parameter or a system 10 parameter. One or more console settings 201 can be manually adjusted (e.g. by a clinician or other operator of system 10) and/or automatically (e.g. by algorithm 251 of system 10) based on the sensor signal.

In some embodiments, one or more of functional elements 109, 119, 139, 209, 229 and/or 309 comprise a pressure sensor that produces a signal related to one or more of: pressure within functional assembly 130; the level of apposition of functional assembly 130 with the intestine; the diameter of the intestine proximate functional assembly 130; muscular contraction of the intestine; pressure within a reservoir 220; pressure within connecting assembly 300; pressure within a lumen of shaft 110; and combinations of two or more of these. One or more console settings 201 can be adjusted (e.g. manually or automatically) based on the pressure sensor signal. In some embodiments, a pressure sensor produces a signal related to the pressure within functional assembly 130, console 200 delivers and/or extracts fluids to and/or from functional assembly 130 via one or more conduits 111, and console 200 adjusts the volume of functional assembly 130 to maintain pressure in functional assembly 130 below a threshold.

In some embodiments, one or more of functional elements 109, 119, 139, 209, 229 and/or 309 comprise a temperature sensor that produces a signal related to one or more of: temperature of fluid in console 200 (e.g. in one or more reservoirs 220); temperature of elongate shaft 110; temperature of fluid within elongate shaft 110; temperature of functional assembly 130; temperature of fluid within functional assembly 130; temperature of an ablative fluid; temperature of a neutralizing fluid; temperature of tissue proximate the functional assembly; temperature of target tissue; temperature of non-target tissue; and combinations of two or more of these. One or more console settings 201 can be adjusted (e.g. manually or automatically) based on the temperature sensor signal.

In some embodiments, system 10 comprises a sensor (e.g. a functional element 109, 119, 139, 209, 229 and/or 309 comprising a sensor) configured to detect a parameter related to a level of treatment of tissue, such as a parameter selected from the group consisting of color, density and/or saturation of tissue (e.g. a color change to tissue that occurs during ablation or to an injectate 221 present in the tissue during ablation or other treatment); temperature of local tissue and/or temperature of other body tissue; texture, length and/or diameter of villi or other mucosal feature (e.g. as detected via a camera-based sensor, such as when ablation causes a blunting and/or drooping of villi or other intestinal tissue); electrical resistance, impedance and/or capacitance of tissue (e.g. as altered by ablation of tissue); pressure and/or force of peristaltic contractions (e.g. as altered by ablation of tissue); compliance of tissue and/or the entire duodenum in radial and/or axial directions (e.g. as altered by ablation of tissue); chemical composition of film adhered to mucosal tissue (e.g. as altered by ablation); types, quantities and/or locations of bacterial colonies present (e.g. as altered by ablation); and combinations of two or more of these.

In some embodiments, system 10 comprises a sensor (e.g. a functional element 109, 119, 139, 209, 229 and/or 309 comprising a sensor) configured to detect a parameter related to a level of tissue expansion, such as a parameter selected from the group consisting of: color, density and/or saturation related to injected dye or particles which alter tissue appearance (e.g. as determined via a camera-based sensor); temperature of tissue (e.g. that can be altered briefly due to delivery of injectate 221 and/or inflammation response due to injectate 221 delivery); texture, length and/or diameter of villi or mucosal features (e.g. as determined via a camera-based sensor) such as spacing between villi or other intestinal tissue features that can change (e.g. increased spacing, disappearance or reduction of plicae, blebs of injectate 221 present) due to submucosal tissue expansion; electrical resistance, impedance and/or capacitance of tissue (e.g. as altered by delivery of injectate 221); pressure and/or force of peristaltic contractions (e.g. as altered by delivery of injectate 221); compliance of tissue and/or the entire duodenum in radial and/or axial directions (e.g. as altered by injectate 221, such as to make tissue more compliant until the muscularis layer is contacted); chemical composition of film adhered to mucosa (e.g. as altered by injectate 221, such as when injectate 221 creates a biologic response that is detectable); types, quantities and/or locations of bacterial colonies present; and combinations of two or more of these.

In some embodiments, system 10 comprises a sensor (e.g. a functional element 109, 119, 139, 209, 229 and/or 309 comprising a sensor) configured to assess engagement of port 137 with tissue (e.g. to determine if adequate engagement is present during a tissue expansion or tissue ablation step in which vacuum is applied to port 137 to engage port 137 with tissue). In some embodiments, a sensor is positioned to detect injectate in a conduit 111 of catheter 100 in which the vacuum is applied. The detector can comprise an optical sensor, and/or a window which is visualizable by an operator (e.g. to see injectate that is recovered), such as when the injectate comprises visible material.

In some embodiments, one or more functional elements 109, 119, 139, 209, 229 and/or 309 comprises one or more temperature sensors that produces a signal related to a first temperature representing the temperature of ablative fluid delivered to functional assembly 130 and a second temperature related to the temperature of fluid extracted from functional assembly 130. In these embodiments, system 10 can be configured to assess (e.g. via algorithm 251) the effect (e.g. quantity) of tissue treated (e.g. depth of tissue ablated), such as by analyzing the first temperature and the second temperature (e.g. a comparison of the two). In some embodiments, the first and/or second temperature is measured by one or more sensors of connecting assembly 300 (e.g. two or more functional elements 309 comprising thermistors or other temperature sensors) and/or one or more sensors of catheter 100 (e.g. two or more functional elements 109, 119 and/or 139 comprising thermistors or other temperature sensors).

In some embodiments, one or more of functional elements 109, 119, 139, 209, 229 and/or 309 comprise a sensor configured to provide a signal related to lumen diameter information. In these embodiments, the sensor can comprise a sensor selected from the group consisting of: pressure sensor; optical sensor; sound sensor; ultrasound sensor; strain gauge; electromagnetic sensor; an imaging device such as a camera; and combinations of two or more of these. One or more console settings 201 can be adjusted (e.g. manually or automatically) based on the lumen diameter information.

In some embodiments, one or more of functional elements 109, 119, 139, 209, 229 and/or 309 comprise a sensor including an imaging device configured to provide a signal related to image information. The imaging device can comprise a device selected from the group consisting of: visible light camera; infrared camera; endoscope camera; MRI; Ct Scanner; X-ray camera; PET Scanner; ultrasound imaging device; and combinations of two or more of these. In these embodiments, controller 250 or another assembly of system 10 can comprise signal processor 252 and/or algorithm 251, each of which can be configured to analyze the image information provided by the imaging device. One or more console settings 201 can be adjusted (e.g. manually or automatically) based on the image information. Based on the image information, system 10 can be configured to modify a console setting 201 to cause an event selected from the group consisting of: stopping delivery of fluid and/or energy to functional assembly 130; delivering additional fluid into functional assembly 130 and/or into tissue; delivering neutralizing fluid into functional assembly 130 and/or into tissue; adjusting the pressure of functional assembly 130; adjusting the volume of functional assembly 130; and combinations of two or more of these.

In some embodiments, one or more functional elements 109, 119, 139, 209, 229 and/or 309 comprise a filter (e.g. a hydrophobic filter) positioned in a fluid pathway of system 10. The filter can be positioned between a sensor and the fluid pathway. In these embodiments, the associated functional element 109, 119, 139, 209, 229 and/or 309 can further comprise a valve, such as a valve configured to vent the fluid pathway proximate the filter.

In some embodiments, one or more of functional elements 109, 119, 139, 209, 229 and/or 309 comprise a sensor configured to detect gas-bubbles, such as a gas bubble present in one or more of conduits 111, 211, 212 and/or 311 and/or a gas bubble present in functional assembly 130. In some embodiments, one or more de-gassing procedures are performed on one or more components of system 10, and the one or more gas-bubble detector based functional elements 109, 119, 139, 209, 229 and/or 309 are used to confirm that the de-gassing procedure is adequately completed and/or to indicate a de-gassing procedure should be performed.

In some embodiments, functional assembly 130 and/or other components of catheter 100, connecting assembly 300 and/or console 200 are configured to enhance mixing of one or more fluids within functional assembly 130 (e.g. one or more functional element 139 comprising a fluid mixing element). In some embodiments, one or more functional elements 139 within functional assembly 130 comprise a baffle configured to improve fluid mixing and/or occupy a volume (e.g. a baffle positioned within functional assembly 130). In some embodiments, one or more functional elements 139 comprise an expandable and/or compressible baffle. These baffles can be configured to “take up” volume within functional assembly 130, such as to decrease the amount of fluid (e.g. ablative fluid) delivered into functional assembly 130 during a tissue ablation and/or tissue expansion procedure. The baffles can be configured to reduce rise times or fall times of temperatures associated with functional assembly 130 (e.g. reduce rise times or fall times to or from ablative temperatures, respectively, during a tissue ablation procedure). The baffles can be configured to take up volume in between two or more ports 137, such as to minimize the overall diameter of a catheter 100 configured as a tissue expansion device.

In some embodiments, a first conduit 111 can comprise an inflow tube configured to at least deliver fluid to functional assembly 130. A second conduit 111 can surround the first conduit 111, and an opening on the proximal end of the second (outer) conduit 111 can be closed off (e.g. a proximal end of second conduit 111 positioned near the proximal end of functional assembly 130). The distal end of the second conduit 111 can extend past the midpoint of functional assembly 130 but terminate proximal to the distal end of functional assembly 130, forming a collar around the inner first conduit 111 that channels the flow from the first conduit 111 to the distal portion of functional assembly 130, and improving mixing within all of the internal volume of functional assembly 130.

In some embodiments, catheter 100 comprises one or more insulating elements configured to avoid transfer of energy from shaft 110 to tissue, such as an insulating element comprising a full or partial layer of shaft 110 that comprises thermally insulating material and/or an insulating element comprising one or more conduits 111 which contain circulating fluid configured to dissipate heat from shaft 110.

Shaft 110 of catheter 100 can comprise one or more coatings, coating 118, along all or a portion of its outer and/or inner surfaces. In some embodiments, coating 118 is positioned on at least a portion of the outer surface of shaft 110, and the coating 118 is configured to prevent or otherwise reduce inadvertent translation of catheter 100 through the intestine (e.g. an anti-migration coating configured to reduce undesired translation and/or rotation of catheter 100). Alternatively or additionally (e.g. on a different portion), coating 118 can comprise a lubricous coating. In some embodiments, coating 118 is positioned on one or more lumens of shaft 110, such as a lubricous coating configured to assist in the translation of one or more filaments within the lumen. In some embodiments, coating 118 comprises a coating positioned on at least a portion of shaft 110 and selected from the group consisting of: a hydrophilic coating (e.g. to improve lubricity); a coating comprising bumps (e.g. atraumatic projections configured to roughen a surface to reduce friction); a coating comprising a surface exposed to grit blasting (e.g. to roughen a surface to reduce friction); an insulative coating: parylene; PTFE; PEEK; a coating comprising a colorant (e.g. to improve or otherwise improve visibility of shaft 110 in-vivo); and combinations of two or more of these. In some embodiments, coating 118 comprises a coating positioned on at least a portion of functional assembly 130 (e.g. on at least a portion of a balloon 136) and selected from the group consisting of: a lubricous coating; a surface roughening coating; a silicone coating; an insulative coating; and combinations of two or more of these.

In some embodiments, multiple conduits 111 are in fluid communication with functional assembly 130 (e.g. to simultaneously or sequentially inflate and/or deflate functional assembly 130) and/or port 137 (e.g. to simultaneously or sequentially provide a vacuum to port 137). In these embodiments, simultaneous and/or redundant delivery or extraction of fluids (e.g. application of a vacuum) can be initiated based on the signal provided by one or more sensors of system 10. For example, if a sensor detects a first conduit 111 is fully or partially occluded, the second conduit 111 can be used to additionally or alternatively deliver and/or extract fluids.

In some embodiments, system 10 is configured to maintain the pressure of functional assembly 130 relative to a threshold (e.g. pressure is maintained below a pressure threshold, above a pressure threshold, and/or within a threshold comprising a range of pressures), such as during treatment and/or diagnosis of target tissue of the intestine (e.g. during a tissue expansion and/or tissue ablation procedure). Functional assembly 130 can comprise a balloon 136 comprising a compliant balloon; a non-compliant balloon; a pressure-thresholded balloon; and/or a balloon comprising compliant and non-compliant portions, as described herein. Pressure can be maintained at a particular pressure or within a particular range of pressures by monitoring one or more sensors of system 10, such as sensor 139b and/or a sensor-based functional element 119, 109, 209 and/or 229. A lower pressure threshold can comprise a pressure of at least 0.2 psi, and/or no more than 1.0 psi, such as a lower pressure threshold of 0.3 psi, 0.5 psi or 0.7 psi. A lower pressure threshold can be selected to ensure sufficient contact of functional assembly 130 with tissue. An upper pressure threshold can comprise a pressure of at least 0.8 psi, and/or no more than 5.0 psi, such as an upper pressure threshold of 1.0 psi, 1.2 psi, 2.5 psi or 4.0 psi. An upper pressure threshold can be selected to avoid damage to tissue, such as damage to an outer layer of intestinal tissue (e.g. a serosal layer of the intestine). Pressure can be monitored such that console 200 can modulate or otherwise control one or more inflow and/or outflow rates of fluid delivered to and/or extracted from functional assembly 130. Pressure can be monitored to maintain flow rates to or from functional assembly 130 to a minimum rate of at least 250 ml/min, 500 ml/min, 700 ml/min or 750 ml/min. In some embodiments, pressure is determined by a sensor positioned outside of balloon 136, such as when pressure is maintained in functional assembly within a narrow range of pressures, such as at a pressure of between 1.05 psi and 0.55 psi. In these embodiments, a luminal sizing step can be avoided. In some embodiments, system 10 comprises one or more catheters 100 and/or one or more functional assemblies 130, such as to provide an array of functional assemblies 130 with different lengths and/or diameters. In these embodiments, the upper and/or lower pressure thresholds can be independent of functional assembly 130 size.

In some embodiments, conduits 111 comprise an inflow tube and an outflow tube fluidly connected to functional assembly 130. Fluid can be delivered to functional assembly 130 by console 200 via one or more conduits 111 at various flow rates, such as flow rates up to 500 ml/min, 1000 ml/min, 1500 ml/min, 2000 ml/min and/or 2500 ml/min. Fluid can be extracted from functional assembly 130 by console 200 via one or more conduits 111 at various flow rates, such as flow rates up to 500 ml/min, 750 ml/min, or 1000 ml/min.

In some embodiments, treatment element 139a can comprise fluid at a sufficiently high temperature to ablate tissue (such as liquid above 60° C. or steam). Delivery of superheated fluid through a conduit 111 can be performed, such as when functional element 119 comprises an orifice configured to cause the superheated fluid to boil upon entering functional assembly 130, providing steam at 100° C. Delivery of cooled fluids through a conduit 111 can be performed. In some embodiments, a fluid (cooled or otherwise) is introduced through a conduit 111 and through a functional element 119 comprising a valve, such that expansion of the fluid into functional assembly 130 results in a cooling effect.

In some embodiments, system 10 and catheter 100 are constructed and arranged to fill functional assembly 130 with neutralizing (e.g. chilled) fluid, and then thermally prime a first conduit 111 with ablative (e.g. hot) fluid, when the first conduit is positioned in a retracted state (e.g. preventing or otherwise reducing heating of functional assembly 130). Subsequently, the first conduit 111 is advanced (i.e. first conduit 111 is constructed and arranged as a translatable conduit) and ablative fluid is introduced into functional assembly 130, allowing functional assembly 130 to be in a fully or partially expanded state prior to fluid at an ablative temperature residing in functional assembly 130 and avoiding undesired “partial ablative contact” of functional assembly 130 with tissue. Another advantage of this configuration is that functional assembly 130 can be checked for leaks with non-ablative fluid prior to one or more subsequent steps (e.g. each ablation step).

In some embodiments, functional assembly 130 is constructed and arranged to both expand tissue (e.g. expand submucosal tissue) and treat target tissue (e.g. treat duodenal mucosal tissue), such as is described herebelow in reference to multi-function catheter 40 of FIG. 2. For example, functional assembly 130 can comprise fluid delivery element 139c which can be positioned to deliver fluid into tissue that has been drawn into (e.g. upon application of a vacuum) port 137, to expand one or more layers of tissue (e.g. one or more layers of submucosal tissue). Functional assembly 130 can further comprise treatment element 139a which can comprise ablative fluid which can be introduced into functional assembly 130 and/or an energy delivery element configured to deliver energy to tissue (e.g. RF energy, light energy, sound energy, chemical energy, thermal energy and/or electromagnetic energy), each configured to perform a therapeutic treatment on target tissue.

In some embodiments, system 10 and catheter 100 are configured to both expand tissue (e.g. expand submucosal tissue of the intestine) and treat target tissue (e.g. treat mucosal tissue of the intestine proximate the expanded submucosal tissue). Catheter 100 can comprise a single catheter 100 comprising one or more functional elements 139 configured to collectively expand tissue and treat target tissue, or a first catheter 100a configured to expand tissue and a second catheter 100b configured to treat target tissue. In these embodiments, injectate 221 can comprise a material configured to enhance or otherwise modify a target treatment step. For example, injectate 221 can comprise a conductive fluid (e.g. an electrically conductive fluid), such as saline configured to modify a subsequent target tissue treatment by treatment element 139a in which RF or other electrical energy is delivered to target tissue (e.g. when treatment element 139a comprises an array of electrodes). Similarly, injectate 221 can comprise a chromophore or other light absorbing material and/or a light scattering material configured to modify a subsequent target tissue treatment by treatment element 139a in which light energy is delivered to target tissue (e.g. when treatment element 139a comprises a lens, one or more conduits 111 comprise an optical fiber, and controller 250 comprises an energy delivery unit EDU 260 comprising a laser).

In some embodiments, fluid delivery element 139c comprises a needle with two separate lumens (e.g. two lumens each fluidly connected to a different conduit 111), such that two different materials can be injected into tissue without the two fluids mixing prior to entering the tissue, or at least without the two fluids mixing more than 5 minutes prior to entering the tissue. Alternatively, fluid delivery element 139c can comprise two different needles directed toward a similar area. Injectate 221 can comprise a first material and a second material which form a hydrogel when mixed (e.g. the two materials crosslink to form an absorbable hydrogel). Alternatively or additionally, injectate 221 can comprise water soluble PEG reactive end groups and an amino acid with reactive end groups.

In some embodiments, system 10 includes one or more tools, tool 500 shown. Tool 500 can comprise a vacuum applying tool such as an endoscopic cap. Catheter 100 or a standard endoscopic needle device can inject a material into the wall of the duodenum while the endoscopic cap applies suction to the intestinal mucosa. A needle or other fluid delivery element of catheter 100 (e.g. fluid delivery element 139c) or a needle of a standard endoscopic needle device is delivered into intestinal tissue while the mucosa of the intestine is lifted by tool 500.

In some embodiments, tool 500 comprises an insufflation and/or desufflation tool, such as a catheter comprising a port (e.g. a distal opening) for delivering and/or extracting fluids from the intestine. Tool 500 can be insertable through the working channel of an introduction device 50 (e.g. through an endoscope). Delivery of insufflation fluids can be performed to move tissue away from functional assembly 130 and/or move tissue away from one or more functional elements 139 or other parts of catheter 100. In some embodiments, insufflation is performed to stop or limit a transfer of energy to tissue (e.g. in an emergency or insufflation-controlled ablation step).

In some embodiments, tool 500, catheter 100, introduction device 50 and/or another component of system 10 comprises a pressure-neutralizing assembly constructed and arranged to modify the pressure within a luminal segment of the intestine (e.g. a luminal segment proximate functional assembly 130). In these embodiments, tool 500 and/or catheter 100 can comprise one or more openings or other elements configured as vents, such as to vent the luminal segment to room pressure (e.g. clinical procedure room pressure) or otherwise maintain the pressure in a segment of the intestine below a threshold. In some embodiments, introduction device 50 comprises an endoscope comprising a biopsy port configured to vent the luminal segment to room pressure. The pressure-neutralizing assembly can be configured to extract gas from the intestinal segment, and/or to maintain the pressure within the intestinal segment below a threshold. In some embodiments, venting is activated automatically, such as when a pressure (e.g. as measured by a sensor of the present inventive concepts) reaches a threshold (e.g. as determined by algorithm 251).

In some embodiments, tool 500 comprises a diagnostic tool, such as a diagnostic tool comprising a sensor. Tool 500 can be configured to perform a diagnostic test of the patient and/or a diagnostic test of all or a portion of system 10. Tool 500 can comprise a body-insertable tool. Tool 500 can be constructed and arranged to gather data (e.g. via an included sensor) related to a patient physiologic parameter selected from the group consisting of: blood pressure; heart rate; pulse distention; glucose level; blood glucose level; blood gas level; hormone level; GLP-1 level; GIP Level; EEG; LFP; respiration rate; breath distention; perspiration rate; temperature; gastric emptying rate; peristaltic frequency; peristaltic amplitude; and combinations of two or more of these.

Alternatively or additionally, tool 500 can comprise a tissue marking tool, such as a tissue marking tool configured to be deployed through introduction device 50 (e.g. an endoscope). In some embodiments, system 10 comprises marker 430, which can comprise a dye or other visualizable media configured to mark tissue (e.g. using a needle-based tool 500), and/or a visualizable temporary implant used to mark tissue, such as a small, temporary anchor configured to be attached to tissue by tool 500 and removed at the end of the procedure (e.g. by tool 500) or otherwise passed by the natural digestive process of the patient shortly after procedure completion. Tissue marker 430 can be deposited or deployed in reference to (e.g. to allow an operator to identify) non-target tissue (e.g. a marker positioned proximate the ampulla of Vater to be visualized by an operator to avoid damage to the ampulla of Vater), and/or to identify target tissue (e.g. tissue to be ablated). In some embodiments, tissue marker 430 is deposited or deployed in reference to tissue selected from the group consisting of: gastrointestinal adventitia; duodenal adventitia; the tunica serosa; the tunica muscularis; the outermost partial layer of the submucosa; ampulla of Vater; pancreas; bile duct; pylorus; and combinations of two or more of these.

In some embodiments, system 10 includes a tool 500 comprising a mucus removal assembly constructed and arranged to remove mucus from one or more intestinal wall locations (e.g. a full or partial circumferential segment of intestine), such as to remove mucus prior to a tissue treatment performed by functional assembly 130. Alternatively or additionally, functional assembly 130, one or more functional elements 139 and/or one or more other components of catheter 100 can be constructed and arranged to similarly remove mucus. In some embodiments, mucus is removed mechanically. Alternatively or additionally, mucus is removed by delivery (e.g. via one or more fluid delivery elements 139c) of agent 420 to a tissue surface (e.g. when agent 420 comprises a mucolytic agent).

In some embodiments, system 10 includes pressure neutralizing assembly 72, which can be constructed and arranged to monitor and/or adjust (e.g. automatically or semi-automatically) the pressure within a segment of the intestine, such as to allow one or more therapeutic or diagnostic procedures to be performed by functional assembly 130 at a particular pressure or within a particular range of pressures. Pressure neutralizing assembly 72 can be configured to deliver or extract fluids from a segment of the intestine, such as to perform an insufflation procedure, a desufflation procedure, or to otherwise modify the pressure within the segment of the intestine proximate functional assembly 130.

In some embodiments, system 10 comprises an implantable device, such as implant 192 shown. Implant 192 can comprise a medical device, such as a drug delivery depot or other drug delivery device. Implant 192 can comprise a sensor or sensing device. In some embodiments, system 10 is configured to deliver implant 192 via a functional element 139, such as fluid delivery element 139c (e.g. when fluid delivery element 139c comprises a needle comprising a lumen through which a sensor-based implant 192 can be deployed into tissue such as mucosal tissue, submucosal tissue, other intestinal tissue and/or other tissue of the patient). In some embodiments, system 10 is constructed and arranged to deliver one or more implants 192 into tissue that is not proximate to a significant number of pain-sensing nerves. In some embodiments, implant 192 can comprise a sensor configured to measure a physiologic parameter selected from the group consisting of: blood pressure; heart rate; pulse distention; glucose level; blood glucose level; blood gas level; hormone level; GLP-1 level; GIP Level; EEG; LFP; respiration rate; breath distention; perspiration rate; temperature; gastric emptying rate; peristaltic frequency; peristaltic amplitude; and combinations of two or more of these.

In some embodiments, implant 192 comprises a sensor, such as a sensor configured to be implanted in the submucosal tissue of the intestine. In some embodiments, catheter 100 is configured to deploy implant 192 into tissue via a fluid delivery element 139c and/or another functional element of catheter 100. Implant 192 can comprise a sensor configured to produce a signal related to a physiologic parameter related to the concentration of a material selected from the group consisting of: fat, sugar (e.g. glucose or fructose); protein; one or more amino acids; and combinations of two or more of these. In some embodiments, implant 192 comprises a wireless communication element, such as an RF or infrared element configured to transmit information (e.g. to a receiving component of system 10). System 10 can be configured to analyze the received information, such as an analysis performed by algorithm 251 used to manage obesity, insulin resistance and/or Type 2 diabetes.

In some embodiments, system 10 includes body core cooling device 73, which can be constructed and arranged to cool the patient's body temperature (e.g. core body temperature). For example, body core cooling device 73 can be configured to cool one or more portions of the patient during a tissue ablation or other procedure performed by catheter 100 as described herein. Body cooling device 73 can be constructed and arranged to cool the patient's blood (e.g. via an external blood circulation circuit), intestine, and/or other body location, such as by extracting heat from one or more body locations. Body cooling device 73 can comprise an elongate shaft for positioning in the esophagus. In some embodiments, body cooling device 73 is used to reduce the patient's core body temperature prior to performance of one or more ablation procedures performed by functional assembly 130 of catheter 100.

In some embodiments, one or more reservoirs 220 and/or one or more pumping assemblies 225 are constructed and arranged to provide a cryogenic gas or other cryogenic fluid to functional assembly 130, such as to perform a cryogenic ablation of target tissue and/or to cool target tissue that has been heated above body temperature. Cryogenic gas can be delivered through smaller diameter conduits 111 than would be required to sufficiently accommodate a liquid ablative or neutralizing fluid, which correlates to a reduced diameter of shaft 110. Balloon 136 can comprise a compliant balloon (e.g. a highly compliant balloon). Balloon 136 can be fluidly connected to multiple fluid transport conduits 111, singly or collectively providing inflow (i.e. delivery) and/or outflow (i.e. extraction) of the cryogenic gas. System 10 can be configured to control the pressure within balloon 136, such as at a pressure sufficient, but not much greater than that which would be required to simply inflate balloon 136. A highly compliant balloon 136 can be configured to reduce or avoid the need for a luminal sizing step to be performed. Temperature seen by the target tissue is driven by the temperature of the fluid in balloon 136. During treatment (i.e. cryogenic ablation) the pressure in balloon 136 can be maintained at a pressure at or below 20 inHg, such as below 18 inHg, 15 inHg or 10 inHg.

In some embodiments, pumping assembly 225 comprises at least two pumping assemblies 225 configured to propel fluid out of (i.e. extract fluid from) functional assembly 130 and/or another component of catheter 100, such as two pumping assemblies 225 which operate simultaneously during the performance of a functional assembly 130 drawdown procedure (e.g. an emergency radial contraction of functional assembly 130 that is initiated during an undesired situation, such as an emergency drawdown procedure initiated when a leak is detected). In some embodiments, two pumping assemblies 225 are configured to deliver fluid to functional assembly 130 (e.g. to balloon 136 and/or one or more fluid delivery elements 139c) or other component of catheter 100. In these embodiments, simultaneous fluid delivery can also be performed when a leak is detected, such as to simultaneously deliver a neutralizing fluid to tissue being undesirably exposed to ablative fluid. Alternatively or additionally, a second pumping assembly 225 can be configured to begin fluid delivery and/or fluid extraction when the failure of a first pumping assembly 225 is detected. Two or more pumping assemblies 225 can be fluidly attached to one or more fluid transport conduits 211.

In some embodiments, console 200 is constructed and arranged to maintain a minimum volume (e.g. a minimum level of fluid) of one or more reservoirs 220. In some embodiments, console 200 is constructed and arranged to disable a pump 225 if an undesired condition is detected, such as by a signal recorded by a functional element 229a and/or 229b that comprises a sensor configured to monitor one or more system parameters (e.g. temperature, pressure, flow rate, and the like).

In some embodiments, console 200 is constructed and arranged to limit a treatment time or to limit another treatment parameter. In these embodiments, the treatment parameter can be limited by software, such as software of algorithm 251 and/or controller 250. Alternatively, the treatment parameter can be limited by hardware (e.g. a hardware-based algorithm 251), such as hardware of controller 250 such as a temperature controlled functional element which turns off a pumping assembly 225 and/or otherwise prevents or reverses energy being delivered by a functional assembly 130 of catheter 100.

In some embodiments, system 10 is constructed and arranged (e.g. via algorithm 251) to adjust one or more treatment parameters, such as an adjustment based on the expanded size of a functional assembly 130, such as when system 10 comprises multiple catheters 100, each comprising a different expanded size of its functional assembly 130. In these embodiments, system 10 can be constructed and arranged to adjust one or more treatment parameters selected from the group consisting of: temperature of ablative fluid; volume of ablative fluid; pressure of ablative fluid; amount of energy delivered such as peak amount of energy delivered and/or cumulative amount of energy delivered; duration of treatment; amount of fluid delivered into tissue (e.g. during a tissue expansion procedure or a tissue ablation procedure); and combinations of two or more of these.

In some embodiments, console 200 is constructed and arranged to provide a first fluid at an ablative temperature, and a second fluid at a neutralizing temperature. For example, a first fluid can be provided by a first reservoir 220 such that the first fluid enters functional assembly 130 at a sufficiently high temperature to ablate tissue, such as at a temperature above 44° C. or above 60° C. A second fluid can be provided by a second reservoir 220 such that the second fluid enters functional assembly 130 at a neutralizing temperature below body temperature, such as a temperature between room temperature and body temperature, or a temperature below room temperature. Alternatively, an ablative fluid can comprise a fluid of sufficiently low temperature to ablate tissue (e.g. below 5° C.), and an associated neutralizing fluid can comprise a warmer fluid configured to reduce the tissue damaging effects of the ablative fluid, as described herein. In some embodiments, a neutralizing fluid is provided to functional assembly 130 prior to and/or after delivery of ablative fluid to functional assembly 130, as described in detail herebelow.

An ablative fluid and a neutralizing fluid can be transported to functional assembly 130 via the same or different conduits 111. Fluid can be extracted from functional assembly 130 via the same or different conduits used to deliver the first fluid and/or the second fluid. In some embodiments, conduits 111 used to deliver and/or extract an ablative fluid or a neutralizing fluid are configured to be translated (e.g. advanced and/or retracted), such that their distal end position within or otherwise relative to functional assembly 130 can be varied. In some embodiments, one or more conduits 111 and/or functional assembly 130 can be thermally primed prior to treating target tissue. In some embodiments, ablative fluid and/or neutralizing fluid is provided to functional assembly 130 in a recirculating manner. Alternatively, ablative fluid and/or neutralizing fluid can be provided to functional assembly 130 as a bolus (non-circulating volume of fluid). In some embodiments, functional element 119 comprises one or more valves constructed and arranged to control the flow of fluid through one or more conduits 111. In recirculating fluid embodiments, a conduit 111 supplying fluid can be manually or automatically changed to a fluid extraction conduit, such as when a separate conduit 111 is configured to normally extract fluid from functional assembly 130 becomes occluded, when a conduit 111 or functional assembly 130 begins to leak, or otherwise when it is desired to radially compact functional assembly 130 at an accelerated rate.

In some embodiments, at least a first conduit 111a provides ablative fluid to functional assembly 130 while at least a separate conduit 111b simultaneously withdraws ablative fluid from functional assembly 130, such as to recirculate ablative fluid within functional assembly 130. In these embodiments, functional assembly 130 can be radially expanded (e.g. initially or after a radial compacting step), by filling functional assembly 130 (e.g. with ablative fluid, neutralizing fluid and/or other fluid) by using both first conduit 111a and second conduit 111b.

In some embodiments, balloon 136 comprises at least a porous portion or a portion otherwise constructed and arranged to allow material contained within balloon 136 to pass through at least a portion of balloon 136. In these embodiments, injectate 221 can comprise a material configured to pass through at least a portion of balloon 136, such as a conductive gel material configured to modify energy delivery, such as when treatment element 139a comprises one or more electrodes configured to delivery RF energy to target tissue. In other embodiments, agent 420 comprises one or more agents configured to be delivered into balloon 136 and to pass through at least a portion of balloon 136 and into the intestine.

In some embodiments, system 10 is constructed and arranged to deliver fluid into functional assembly 130 at a flow rate of at least 500 ml/min, at least 1000 ml/min, at least 2000 ml/min, or at least 2500 ml/min. In some embodiments, system 10 is constructed and arranged to extract fluid from functional assembly 130 at a flow rate of at least 500 ml/min, at least 750 ml/min, or at least 1000 ml/min. In some embodiments, system 10 is constructed and arranged to remove and extract fluids at approximately the same flow rate. In some embodiments, fluid in console 200 is provided to catheter 100 at a temperature of at least 60° C., 70° C. or 80° C. In some embodiments, system 10 is configured to treat at least three axial segments of intestinal tissue, such as at least three axial segments of tissue treated with a heat ablation and at least one cooling step (e.g. a cooling step performed prior to and/or after the heat ablation step).

In some embodiments, functional assembly 130 is positioned in an axial segment of intestine, expanded to a diameter less than the average diameter of the axial segment, and activated (e.g. to deliver energy to tissue and/or fluids to tissue) during a contraction of the intestine. In these embodiments, the contraction of the intestine can be one or more of: a (natural) peristaltic contraction; a contraction caused by stimulation (e.g. electrical or chemical stimulation by catheter 100 and/or tool 500); a contraction caused during a desufflation procedure; and combinations of two or more of these. Contraction of the intestine can comprise a desufflation procedure performed by a device selected from the group consisting of: catheter 100; an endoscope or other body introduction device 50; a second catheter inserted into the intestine; and combinations of two or more of these.

Functional assembly 130 can be configured to perform a medical procedure (e.g. a tissue expansion procedure and/or a tissue ablation or other tissue treatment procedure) on multiple axial segments of intestinal tissue. Two or more of the multiple axial segments can be treated sequentially and/or simultaneously. The two or more of the multiple axial segments can be relatively proximate each other, such as to share common boundaries or avoid significant gaps in untreated tissue. The multiple axial segments can comprise partial or full circumferential segments of intestinal tissue. The multiple axial segments can cumulatively comprise at least 3 cm in length or at least 6 cm in length, such as when between one and six treatments (e.g. between two and six treatments) are performed (e.g. functional assembly 130 is repositioned between one and five times). The multiple axial segments can cumulatively comprise a length of at least 9 cm, such as when between two and nine treatments are performed (e.g. functional assembly 130 is repositioned between one and eight times). In these embodiments, system 10 can be configured to treat diabetes, such as Type 2 diabetes. In some embodiments, system 10 is constructed and arranged to treat diabetes as described in applicant's co-pending U.S. patent application Ser. No. 15/406,572 (Attorney Docket No. 41714-713.301; Client Docket No. MCT-029-US), entitled “Methods and Systems for Treating Diabetes and Related Diseases and Disorders”, filed Jan. 13, 2017, the content of which is incorporated herein by reference in its entirety for all purposes.

In some embodiments, system 10 is configured to initially expand functional assembly 130, with a fluid at a non-ablative temperature (e.g. a fluid configured to cool tissue without ablating it), after which a fluid at an ablative temperature can be introduced into functional assembly 130 (e.g. a fluid at sufficiently high temperature to ablate tissue).

In some embodiments, catheter 100 and/or another device of system 10 comprises an anchoring element, such as when port 137 is configured to fixedly engage tissue when a vacuum is applied to port 137 (e.g. via one or more conduits 111). Alternatively or additionally, inflation of balloon 136 can be used to anchor functional assembly 130 at a particular intestinal location. One or more functional elements 139 can comprise an anchor element, such as a high friction coating or surface treatment, or an extendable barb.

In some embodiments, system 10 is constructed and arranged to allow an operator to position the functional assembly within an axial segment of the intestine and perform a first procedure on intestinal tissue with functional assembly 130. System 10 is further constructed and arranged to anchor functional assembly 130 (prior to, during and/or after the first procedure). Subsequent to the performance of the first procedure and the anchoring of functional assembly 130, a second procedure is performed. The first procedure can comprise a tissue expansion procedure. The second procedure can comprise a tissue ablation procedure, such as a tissue ablation procedure which ablates mucosal tissue within or otherwise proximate previously expanded submucosal tissue. Repeating of the three steps (i.e. the first procedure, the anchoring of functional assembly 130, and the second procedure) can be performed at additional locations within the intestine.

As described herein, in some embodiments, catheter 100 or another device of system 10 such as catheter 30 of system 10 of FIG. 2, is constructed and arranged to perform a luminal sizing measurement (e.g. a measurement in which diameter and/or other cross sectional geometry is quantified), and produce luminal size information. In these embodiments, system 10 can include multiple catheters 100, one of which is selected and/or adjusted based on the luminal size information. Alternatively or additionally, system 10 can be configured to adjust one or more system parameters based on the luminal size information, such as a console setting 201 selected from the group consisting of: volume of fluid delivered into functional assembly 130; flow rate of fluid delivered into functional assembly 130; temperature of fluid delivered into functional assembly 130; pressure of functional assembly 130; and combinations of two or more of these.

In some embodiments, console 200 and system 10 are constructed and arranged to maintain functional assembly 130 of catheter 100 at or below a target level of a functional assembly 130 parameter, such as at or below a target diameter, pressure and/or volume for functional assembly 130. In some embodiments, functional assembly 130 is maintained below a target pressure of 0.9 psi (e.g. during a tissue expansion, tissue ablation and/or other tissue treatment step).

In some embodiments, catheter 100 and system 10 are constructed and arranged to compensate for muscle contraction of the intestine (e.g. peristalsis within the intestine). For example, algorithm 251 can be configured to actively regulate a functional assembly 130 parameter (e.g. diameter, pressure within and/or flowrate to and/or from), such as when algorithm 251 anticipates, recognizes and/or compensates for muscular contraction of the intestine. In some embodiments, expansion of functional assembly 130 can be timed to occur during the bottom (lower range) of a muscular contraction (e.g. peristalsis) cycle.

In some embodiments, catheter 100 and system 10 are constructed and arranged to perform a medical procedure in the intestine that is synchronized with one or more muscular contractions of the intestine, such as one or more peristaltic contractions used to contact intestinal wall tissue with an expanded or partially expanded functional assembly 130.

In some embodiments, system 10 is constructed and arranged to size a lumen of a first axial segment of the intestine. System 10 can be further constructed an arranged to subsequently perform a tissue expansion of a portion of the first axial segment (e.g. a full or partial circumferential segment of the submucosa of the axial segment), by injecting fluid (e.g. a fixed volume of fluid) into tissue within or proximate the first axial segment. System 10 can be further constructed and arranged to subsequently perform a luminal sizing measurement of the first axial segment. System 10 can be further constructed and arranged to subsequently perform a target tissue treatment of the first axial segment (e.g. a treatment of a full or partial circumferential segment of the mucosal tissue of the first axial segment). The treatment performed by system 10 can comprise one or more treatment parameters (e.g. one or more ablation parameters) that are based on the luminal sizing measurement performed after tissue expansion and determined via algorithm 251.

As described hereabove, system 10 can be constructed and arranged to ablate or otherwise treat tissue with an expanded functional assembly 130 that is smaller than the native lumen diameter of an axial segment of intestine. The amount of fluid injected to expand tissue (e.g. submucosal tissue) can be determined in a closed-loop manner to achieve a post-expansion lumen size with a specific diameter along one or more axial segments of the intestine (e.g. the duodenum). System 10 can comprise a single functional assembly 130 configured to treat (e.g. ablate) multiple axial segments of intestine, each with a pre-expanded tissue layer (e.g. submucosal tissue layer expanded to a diameter approximating or otherwise related to the diameter of the expanded functional assembly 130). System 10 can comprise a functional assembly 130 configured to treat (e.g. ablate) multiple axial segments of intestine that are selected prior to the performance of tissue layer expansion, such as to reduce overall procedure time and/or time between tissue expansion and tissue treatment. System 10 can be constructed and arranged such that the difference between the native intestinal lumen diameter and the post-tissue expansion lumen diameter is known, such as to confirm acceptability of the tissue expansion step(s) prior to an ablation step being performed. System 10 can be constructed and arranged to eliminate one or more sizing steps, as described hereabove.

In some embodiments, system 10 is constructed and arranged to perform a medical procedure comprising a tissue treatment procedure for treating a patient disease or disorder, and the amount of tissue treated is based on the severity of the patient's disease or disorder (e.g. amount of tissue treated is proportional to the severity). In some embodiments, the disease treated is diabetes, and the severity is determined by measuring one or more of: HbA1c level; fasting glucose level; and combinations of two or more of these. In some embodiments, algorithm 251 is configured to determine the amount of tissue to be treated based on the severity of the patient's disease or disorder.

In some embodiments, system 10 is constructed and arranged to (e.g. via algorithm 251) introduce fluid into functional assembly 130 (e.g. into a balloon 136 of functional assembly 130) until sufficient apposition against an intestinal wall is achieved (e.g. as determined by a pressure measurement and/or image analysis provided by a sensor of the present inventive concepts). Subsequently, fluid is extracted from functional assembly 130 (e.g. until a second, lesser volume of fluid resides within functional assembly 130), after which the intestinal wall is contracted (e.g. via desufflation as described herein) such that the intestinal wall again contacts functional assembly 130.

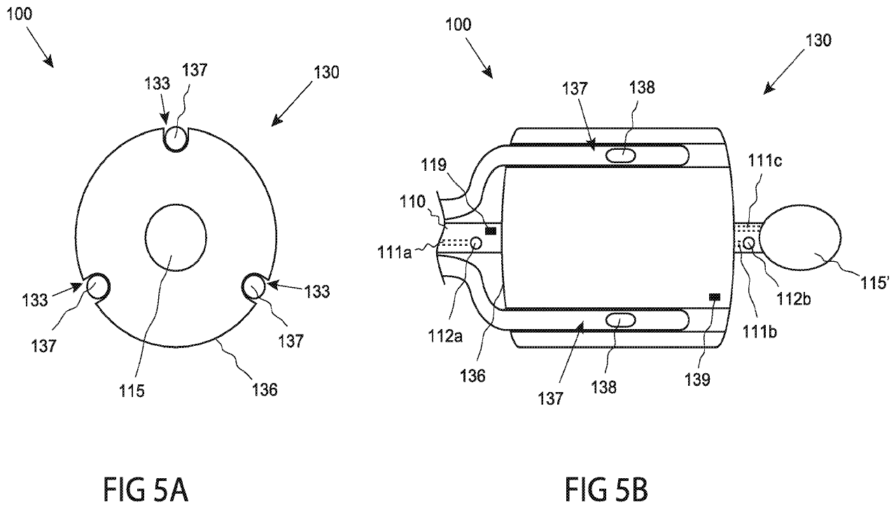

In some embodiments, desufflation is accomplished by applying vacuum to a port (e.g. one or more ports configured to remove fluid from the intestine, such as port 137), one or more ports of shaft 110 proximal or distal to functional assembly 130 (e.g. port 112a and/or 112b described herebelow in reference to FIG. 5B) and/or a lumen of an endoscope or other introduction device 50.

In some embodiments, system 10 is constructed and arranged to expand tissue by delivering injectate 221 into tissue (e.g. submucosal tissue of the intestine) with fluid delivery element 139c. System 10 can be constructed and arranged to deliver injectate 221 at a constant or varied rate, in open loop or closed loop delivery configurations. In some embodiments, system 10 is configured to deliver fluid at an elevated flow rate and/or at an elevated pressure, such as with a flow rate and/or pressure which decreases over time. System 10 can be constructed and arranged to monitor one or more pressures achieved during delivery of injectate 221 into tissue. System 10 can be configured to measure a pressure using a pressure sensor-based functional element 109, 119, 139, 209, 229 and/or 309. Alternatively or additionally, system 10 can comprise a sensor positioned in tissue proximate the tissue to be expanded. In some embodiments, catheter 100 comprises multiple fluid delivery elements 139c, such as an array of three fluid delivery elements 139c equally spaced about functional assembly 130. In these embodiments, injectate 221 can be delivered into tissue by the multiple fluid delivery elements 139c simultaneously or sequentially. Pressure measured by system 10 can correlate to the quality of tissue expansion, or other tissue expansion parameter. In some embodiments, system 10 regulates delivery of injectate 221 (e.g. by regulation of one or more pumps 225 delivering injectate 221), and/or detects an undesired state in the delivery of injectate 221, based on pressure measured by system 10. System 10 can be configured to confirm that during delivery of injectate 221, a proper pressure increase occurs in the expanded tissue, within functional assembly 130 and/or at another system 10 location. The pressure at a first location can be measured directly (e.g. via a pressure sensor-based functional element located proximate the first location, or indirectly such as via a pressure sensor-based functional element located at a second location whose pressure can be correlated to the pressure at the first location, as described herein for measurement of pressure, temperature and/or any system 10 parameter). System 10 can prevent a pressure threshold from being surpassed at one or more locations, such as to prevent an undesired event such as an amount and/or location of expansion of tissue that can have a deleterious effect, such as expansion of serosal tissue of the intestine. In some embodiments, pressure information is processed (e.g. via algorithm 251), such that cumulative pressure information (e.g. time at pressure, pressure change rates, and the like) can be compared to one or more thresholds. In these embodiments, pressure information and/or processed pressure information (herein “pressure information”) can be used to confirm size or geometric shape of expanded tissue, such as to confirm full circumferentiality of a tissue expansion. In some embodiments, system 10 correlates one or more pressure readings below a threshold to an adverse event selected from the group consisting of: fluid delivery element 139c not delivering fluid into the appropriate tissue (e.g. fluid delivery element 139c has not properly penetrated tissue); failure of a functional element such as failure of a functional element comprising a valve; leak in a conduit such as a leak in a conduit 111, 211, 212 and/or 311; and combinations of two or more of these.

In some embodiments, functional assembly 130 is expanded with fluid at a first pressure (e.g. a pressure of approximately 0.5 psi, 0.7, psi or 0.9 psi), and fluid is delivered into tissue by one or more fluid delivery elements 139c (e.g. three fluid delivery elements 139c). During fluid injection, system 10 can monitor pressure (e.g. a sensor of the present inventive concepts monitors pressure within functional assembly 130 and/or within a conduit in fluid communication with functional assembly 130), and if the pressure exceeds a second pressure (e.g. a pressure of at least 0.7 psi, 0.9 psi 1.1 psi, or other pressure greater than the first pressure), system 10 can reduce the pressure within the functional assembly 130 (e.g. reduce the pressure to the first pressure).

In some embodiments, system 10, console 200 and/or catheter 100 are constructed and arranged to perform partial circumferential tissue expansion of one or more axial segments of the GI tract (e.g. less than 360° expansion of submucosal tissue of one or more axial segments of the intestine). In some embodiments, injectate 221 comprises a relatively viscous material and catheter 100 delivers injectate 221 to create focal (i.e. partial circumferential) or multi-focal expansions of tissue (e.g. multiple partial circumferential expansions of submucosal tissue). In some embodiments, a therapeutic restriction or other tissue expansion of the present inventive concepts can comprise two or more focal restrictions created around the circumference of an axial segment of tubular tissue that block more than 50% or more than 75% of the luminal diameter. In some embodiments, a full or near-full circumferential expansion of tissue is created by first expanding (e.g. inflating) a functional assembly 130 and creating one or more focal expansions, subsequently compacting (e.g. deflating) the functional assembly 130, re-expanding (e.g. re-inflating) the functional assembly 130 and creating additional focal expansions between the previously expanded areas to create a substantially circumferential expansion. Prior to re-expanding, functional assembly 130 can be repositioned (e.g. rotated). The compacting and re-expanding can be configured to allow multiple fluid delivery elements 139c to self-reposition during contact with the peaks of the focal expansions (e.g. reposition into valleys in between the focal expansions). Alternatively, the functional assembly 130 can be rotated and/or otherwise repositioned (e.g. via automatic and/or manual repositioning of shaft 110) after the initial focal expansions.

In some embodiments, functional assembly 130 comprises an expanded diameter of a magnitude (e.g. a small enough diameter) configured to accommodate a range of luminal diameters of the small intestine. Desufflation of the duodenum (e.g. using body introduction device 50 and desufflation techniques known to those of skill in the art) can be performed to collapse the inner wall of the intestine onto a fully expanded functional assembly 130. Functional assembly 130 can comprise one or more ports 137 configured to desufflate to collapse the inner wall of the intestine onto functional assembly 130. In some embodiments, shaft 110 or another component of catheter 100 comprises one or more ports configured to perform desufflation, such as ports 112a and/or 112b described herebelow in reference to FIG. 5B. In some embodiments, system 10 comprises a separate desufflation tool (e.g. aspiration tool), such as tool 500 constructed and arranged to extract fluid from a segment of intestine, such as a segment comprising functional assembly 130. In these embodiments, tool 500 can comprise one or more holes, slots, slits or other openings (e.g. positioned in a distal portion of tool 500) that are configured to aspirate fluids from the intestine, such as to collapse the inner wall of the intestine onto a fully expanded functional assembly 130.

In some embodiments, system 10 is configured to work in combination with a patient care practice, such as a patient diet that is maintained prior to and/or after performance of a medical device or diagnostic procedure performed using system 10. For example, a patient diet or other patient practice can be included prior to and/or after a tissue treatment procedure performed by system 10 to slow down healing (e.g. mucosal healing) and/or provide another enhancement to the therapy achieved. In some embodiments, mucosal healing is slowed down by a functional element 139, tool 500 and/or other component of system 10. In some embodiments, regrowth of treated mucosal tissue is enhanced by a pre-procedural and/or post-procedural patient diet. The diet can include: a liquid diet for at least one day; a low sugar diet and/or a low-fat diet for at least one week; a standardized diabetic diet for at least one week; and/or nutritional counseling for at least one week.

In some embodiments, system 10 comprises one or more materials or devices configured to modify tissue healing, such as when catheter 100 is constructed and arranged to treat intestinal mucosa (e.g. duodenal mucosa). For example, injectate 221, or implant 192 can be delivered in and/or proximate target tissue, such as at a time prior to, during and/or after target tissue treatment. In these embodiments, for example, injectate 221, agent 420 and/or implant 192 that is delivered (e.g. by fluid delivery element 139c or another component of catheter 100) can be configured to delay healing of treated tissue in the intestine, such as to provide enhanced therapeutic benefit to the patient and/or prolong the benefit (e.g. enhance or prolong HbA1c reduction). In some embodiments, injectate 221, agent 420 and/or implant 192 comprises a material selected from the group consisting of: a chemotherapeutic agent; a cytotoxic agent; 5Fluorouracil; Mitomycin-c; Tretinoin topical (Retin-A, Retin-A Micro, Renova); Bleomycin; Doxorubicin (Adriamycin); Tamoxifen; Tacrolimus; Verapamil (Isoptin, Calan, Verelan PM); Interferon alfa-2b; Interferon beta 1a (Avonex, Rebif); Interferon alfa-n3 (Alferon N); Triamcinolone (Aristospan, Kenalog-10); Imiquimod (Aldara, Zyclara); and combinations of two or more of these.

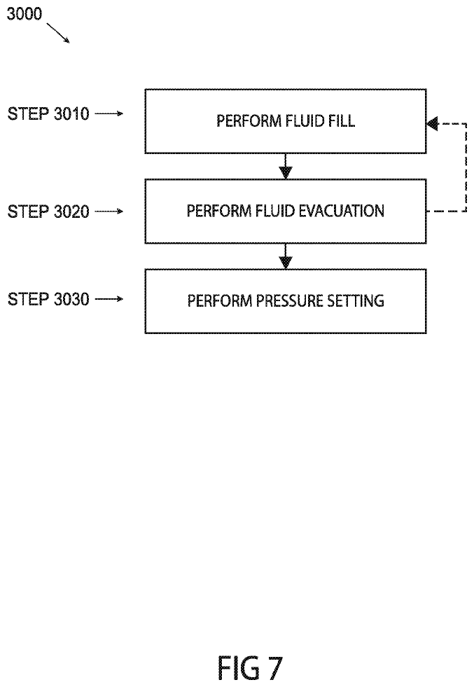

In some embodiments, system 10 of FIG. 1 is configured to perform a medical procedure on a patient as described herebelow in reference to FIG. 7. In some embodiments, system 10 is configured to treat a patient that is taking insulin, such as when catheter 100 is used to treat duodenal mucosa and agent 420 comprises a GLP-1 receptor agonist, and the patient stops taking insulin, as described herein. In these embodiments, the metabolic conditions of these patients can be improved or at least maintained (e.g. HbA1c level or other metabolic condition marker is not made significantly worse by the removal of insulin therapy).

Referring now to FIG. 2, a schematic view of a system and device for performing a medical procedure on the small intestine of a patient is illustrated, consistent with the present inventive concepts. System 10 can comprise one or more components of similar construction and arrangement to similar components of system 10 of FIG. 1 described hereabove. System 10 comprises catheter 100 and console 200. Catheter 100 is constructed and arranged to treat target tissue, such as via the delivery of energy and/or an ablating agent to target tissue. Catheter 100 includes connector 103 which operably attaches to connector 203 of console 200. In some embodiments, system 10 further comprises a tissue expansion device, catheter 20 shown, which is constructed and arranged to expand one or more layers of tissue, such as one or more layers of target tissue and/or one or more layers of tissue proximate target tissue (e.g. one or more layers of safety-margin tissue as described herein). In some embodiments, system 10 further comprises one or more lumen diameter sizing devices, catheter 30, which is constructed and arranged to collect information correlated to the diameter of a portion of tubular tissue (e.g. one, two or more diameters of a GI lumen within and/or proximate target tissue). In some embodiments, system 10 comprises one or more multi-function devices, catheter 40, which is constructed and arranged to perform two or more functions selected from the group consisting of: tissue treatment (e.g. tissue ablation); tissue expansion; luminal diameter sizing; and combinations of two or more of these. In some embodiments, system 10 comprises multi-function catheter 40, and does not include one or more of: catheter 100, tissue expansion catheter 20 and/or sizing catheter 30.

System 10 can further comprise a body introduction device, such as a vascular introducer, laparoscopic port, and/or endoscope, such as endoscope 50a shown. System 10 can further comprise one or more guidewires, such as guidewires 60a and 60b (singly or collectively guidewire 60). In some embodiments, one or more guidewires 60 comprise a guidewire selected from the group consisting of: a Savary-Gilliard® 400 cm guidewire; a Dreamwirem guidewire; a super stiff Jagwirem guidewire; and/or a similar guidewire. In some embodiments, system 10 includes scope attached sheath, sheath 80 shown. Sheath 80 can comprise an elongate hollow tube which attaches (e.g. in a side-by-side manner) at one or more points along endoscope 50a. Sheath 80 can attach to endoscope 50a along a majority of its length. In some embodiments, sheath 80 comprises the Reach® overtube manufactured by U.S. Endoscopy, or similar.

Catheter 100, tissue expansion catheter 20, lumen diameter sizing catheter 30 and multi-function catheter 40 comprise handles 102, 22, 32 and 42, respectively. Handles 102, 22, 32 and 42 each comprise one or more controls, controls 104, 24, 34 and 44, respectively. Controls 104, 24, 34 and 44 are configured to allow an operator to control one or more functions of the associated device, such as a function selected from the group consisting of: inflate or otherwise expand a functional assembly (e.g. functional assembly 130); deliver energy; modify energy delivery; deliver an insufflation fluid; insufflate a portion of the GI tract; desufflate a portion of the GI tract; deliver an injectate (e.g. into tissue and/or onto the surface of tissue); deliver a tissue expanding fluid (e.g. into tissue); steer the distal portion of a shaft; translate a control cable or control rod (hereinafter “control rod”); activate a sensor (e.g. record a signal); activate a transducer; and combinations of two or more of these. In some embodiments, handles 102, 22, 32 and/or 42 comprise a user interface configured to control one or more components of system 10, such as controls 104, 24, 34 and/or 44, respectively, each of which can be constructed and arranged to control operation of one or more of: catheter 100, catheter 20, catheter 30, catheter 40 and/or console 200. In some embodiments, controls 104, 24, 34 and/or 44 comprise one or more user input and/or user output components, such as a component selected from the group consisting of: screen; touchscreen; light; audible transducer such as a beeper or speaker; tactical transducer such as a vibratory motor assembly; a keyboard; a membrane keypad; a switch; a safety-switch 206 such as a foot-activated switch; a mouse; a microphone; and combinations of two or more of these.

Handles 102, 22, 32 and 42 each attach to the proximal end of shafts 110, 21, 31 and 41, respectively. Shafts 110, 21, 31 and 41 each typically comprise a relatively flexible shaft comprising one or more internal lumens or other passageways. Shafts 110, 21, 31 and/or 41 can comprise a lumen, such as lumen 116 of shaft 110 shown, that is sized and configured to perform a function selected from the group consisting of: provide for the delivery or extraction of one or more fluids such as ablation fluids, cooling fluids, insufflation fluids, pneumatic fluids, hydraulic fluids and/or balloon expanding fluids; allow over the guidewire delivery of the associated device; surround an electrical wire providing electrical energy and/or signals; slidingly receive a control shaft or other control filament such as a control filament used to expand or contract a functional assembly (e.g. functional assembly 130) or otherwise modify the shape of a portion of the device; and combinations of two or more of these. Shafts 110, 21, 31 and/or 41 can comprise a braided or otherwise reinforced shaft or they can include one or more portions which are reinforced. Shafts 110, 21, 31 and/or 41 can comprise a multi-layer construction, such as a construction including a braid, a friction-reduced (e.g. PTFE) liner, a thermally insulating layer and/or an electrically insulating layer. Shafts 110, 21, 31 and/or 41 can include a bulbous distal end, such as tip 115 of shaft 110 shown, a circular or elliptical shaped enlarged end configured to improve traversing the innermost tissue of the duodenum or other luminal tissue of the GI tract (e.g. to smoothly advance within a lumen whose walls include villi and/or one or more folds). As described hereabove, shafts 110, 21, 31 and/or 41 can include a guidewire lumen, such as lumen 116 of shaft 110.

Positioned on the distal end or on a distal portion of shafts 110, 21, 31 and 41 is an expandable functional assembly, functional assemblies 130, 25, 35 and 45, respectively. Functional assemblies 130, 25, 35 and 45 are each constructed and arranged to be radially expanded and subsequently radially compacted (each shown in their radially expanded state in FIG. 2), one or more times during use. Each of functional assemblies 130, 25, 35 and 45 can include an expandable element selected from the group consisting of: an inflatable balloon; a radially expandable cage or stent; one or more radially deployable arms; an expandable helix; an unfurlable compacted coiled structure; an unfurlable sheet; an unfoldable compacted structure; and combinations of two or more of these. Each functional assembly 130, 25, 35, and 45 can comprise a balloon, balloons 136, 26, 36, and 46, respectively, as shown. Functional assemblies 130 and/or 45 can each comprise one or more treatment elements, treatment elements 135 and/or 135′ shown, respectively, each an element which can be configured to treat target tissue. Treatment element 135 and/or 135′ (singly or collectively treatment element 135) can be similar to one or more functional elements 139 described hereabove in reference to catheter 100 of FIG. 1.

In some embodiments, catheter 100, tissue expansion catheter 20, lumen diameter sizing catheter 30 and/or multi-function catheter 40, with their functional assemblies 130, 25, 35 and 45 (respectively) in their radially compacted state, are sized and configured to be inserted through a working channel of endoscope 50a and/or sheath 80, after endoscope 50a and/or sheath 80 have been inserted into a patient (e.g. through the mouth and advanced such that their distal end resides in the duodenum or other GI tract location). In some embodiments, catheter 100, tissue expansion catheter 20, sizing catheter 30 and/or multi-function catheter 40 are sized and configured to be inserted through the mouth and into a patient's GI tract alongside endoscope 50a. In some embodiments, catheter 100, tissue expansion catheter 20, lumen diameter sizing catheter 30 and/or multi-function catheter 40 are sized and configured to be inserted into a patient over one or more guidewires 60. For insertion over a guidewire, the shafts 110, 21, 31 and/or 41 and the distal portions of the associated catheter 100, 20, 30 and/or 40 can comprise a distal portion with sufficient length and flexibility to traverse the pylorus and enter the duodenum, while having sufficient column strength, torsional strength, and length to be advanced through the duodenum. In some embodiments, one or more portions of the shafts 110, 21, 31 and/or 41 have variable stiffness (e.g. stiffer in a proximal portion of the shaft) and/or include a lumen configured to accept a stiffening wire or other stiffening mandrel (e.g. a tapered mandrel), such as stiffening wire 67. Alternatively or additionally, stiffening wire 67 can be inserted into endoscope 50a and/or sheath 80, such as to facilitate their advancement through the stomach and into the duodenum. In some embodiments, shaft 110, 21, 31, and/or 41 comprises at least a braided portion. In some embodiments, shaft 110, 21, 31, and/or 41 comprises a tapered portion.

Console 200 can be constructed and arranged in a similar fashion to console 200 of FIG. 1 described hereabove. Console 200 can comprise an operator (e.g. clinician) accessible user interface 205. User interface 205 can comprise one or more user output and/or user input components, such as a component selected from the group consisting of: screen; touchscreen; light; audible transducer such as a beeper or speaker; tactical transducer such as a vibratory motor assembly; a keyboard; a membrane keypad; a switch; a safety-switch, such as switch 206 shown (e.g. a foot-activated switch); a mouse; a microphone; and combinations of two or more of these.

Console 200 can comprise a controller, such as controller 250. Controller 250 can comprise one or more components or assemblies selected from the group consisting of: an electronics module; a power supply; memory (e.g. volatile or non-volatile memory circuitry); a microcontroller; a microprocessor; a signal analyzer; an analog to digital converter; a digital to analog converter; a sensor interface; transducer drive circuitry; software; and combinations of two or more of these. Controller 250 can comprise one or more algorithms 251, which can be constructed and arranged to automatically and/or manually control and/or monitor one or more devices, assemblies and/or components of system 10. Algorithm 251 of controller 250 can be configured to determine one or more tissue expansion, tissue ablation, and/or other tissue treatment parameters. In some embodiments, algorithm 251 processes one or more sensor signals (e.g. signals from functional elements 139, 29, 39 and/or 49 described herein) to modify one or more of: volume of tissue expansion fluid delivered; rate of tissue expansion fluid delivery; temperature of tissue expansion fluid delivery; amount of ablative fluid delivered; rate of ablative fluid delivery; energy delivered; power of energy delivered; voltage of energy delivered; current of energy delivered; temperature of ablative fluid or energy delivered; device and/or treatment element location within the GI tract; functional assembly pressure (e.g. balloon pressure); and combinations of two or more of these. Treatment elements 135 and/or 135′ can deliver energy to a surface of tissue, such as to a delivery zone as described hereabove, which comprises a subset of the target tissue treated by that energy delivery (e.g. due to the conduction of heat or other energy to neighboring tissue). Algorithm 251 can comprise an algorithm configured to determine a delivery zone parameter such as a delivery zone parameter selected from the group consisting of: anatomical location of a delivery zone; size of delivery zone; percentage of delivery zone to receive energy; type of energy to be delivered to a delivery zone; amount of energy to be delivered to a delivery zone; and combinations of two or more of these. Information regarding the delivery zone parameter can be provided to an operator of system 10 (e.g. a clinician), such as via user interface 205. This information can be employed to set a delivery zone parameter, assist the operator in determining the completion status of the procedure (e.g. determining when the procedure is sufficiently complete) and/or to advise the operator to continue to complete a pre-specified area or volume of target tissue. The total area of treatment or number of delivery zones or number of treatments during a particular procedure (any of which can be employed in algorithm 251) can be defined by clinical and/or demographic data of the patient.

Console 200 can comprise one or more reservoirs or other sources of fluid, such as reservoir 220. Reservoir 220 can be configured to provide one or more of: fluid at an ablative temperature (e.g. sufficiently hot or cold to ablate tissue); a treatment neutralizing (e.g. cooling or warming) fluid configured to reduce and/or limit ablative effects; an insufflation fluid, injectate 221 (e.g. similar to injectate 221 described hereabove in reference to FIG. 1); an agent (e.g. agent 420 described hereabove in reference to FIG. 1); and/or another fluid. Console 200 can comprise an energy delivery unit, such as EDU 260, configured to deliver energy to treatment element 135, treatment element 135′, and/or one or more other components of system 10, such as one or more components of catheters 100, 20, 30 and/or 40 (e.g. to functional assemblies 130, 25, 35, and/or 45, respectively). Controller 250, reservoir 220 and/or EDU 260 can be of similar construction and arrangement as controller 250, reservoir 220 and/or EDU 260, respectively, of FIG. 1 described hereabove.

Console 200 can comprise a pressure or other fluid pumping assembly, such as pumping assembly 225 constructed and arranged to deliver positive pressure or vacuum pressure (e.g. any pressure below another pressure) to one or more fluid pathways (e.g. lumens), fluid delivery elements, and/or balloons of system 10. Pumping assembly 225 can be constructed and arranged to provide and/or extract fluid to radially expand and/or radially compact, respectively, one or more expandable assemblies, such as functional assemblies 130, 25, 35 and/or 45 comprising a balloon or other fluid expandable structure (“balloon” herein). Pumping assembly 225 can comprise one or more pumps or other fluid delivery mechanisms, and/or other pressure or vacuum generators. In some embodiments, pumping assembly 225 is constructed and arranged to provide a recirculating ablative fluid (e.g. hot or cold) to catheter 100 and/or catheter 40 (e.g. to balloon 136 and/or 46, respectively). In these embodiments, pumping assembly 225 can be constructed and arranged to further provide a recirculating “neutralizing fluid” (e.g. a cooling or warming fluid, respectively, to counteract the ablative effects of the previously circulated ablative fluid) to balloon 136 and/or 46, respectively. Pumping assembly 225 can be of similar construction and arrangement as pumping assembly 225 of FIG. 1 described hereabove. In some embodiments, pumping assembly 225 is constructed and arranged to deliver injectate 221 to a functional assembly 130, 25, 35 and/or 45, such as an injectate configured to expand tissue and/or to create a therapeutic restriction, as described herein, such as an injectate similar to injectate 221 described hereabove in reference to FIG. 1.

Console 200 includes connector 203, which is operably attached to one or more of: user interface 205 (e.g. safety-switch 206 or another component of user interface 205), controller 250, reservoir 220 and/or pumping assembly 225. Connector 203 is constructed and arranged to operably attach (e.g. fluidly, electrically, optically, acoustically, mechanically and/or otherwise operably attach) to one or more of connectors 103, 23, 33 and 43 of catheters 100, 20, 30 and 40, respectively. Console 200 can be constructed and arranged to deliver fluids and/or energy via connector 203 to one or more of catheters 100, 20, 30 and 40. In some embodiments, an inflation fluid and/or a fluid at an ablative temperature is provided and/or recovered by console 200, such as a fluid at an ablative temperature delivered to functional assembly 130 of catheter 100 and/or functional assembly 45 of catheter 40. In some embodiments, insufflation, pneumatic and/or hydraulic fluids are delivered and/or recovered by console 200 via connector 203. In some embodiments, an injectate 221 is delivered by console 200, such as is described herebelow in reference to tissue expansion catheter 20 and multi-function catheter 40. In some embodiments, one or more control rods (not shown) are translated (e.g. advanced and/or retracted) within one or more lumens or other openings of catheter 100, 20, 30 and/or 40, such as to expand a cage, deploy a radially deployable arm, change the shape of an assembly, translate an assembly, rotate an assembly and/or otherwise control the position, shape and/or configuration of an assembly of system 10.

Console 200 can provide energy to, send information to and/or record and/or receive a signal from one or more other elements of catheter 100, such as functional elements 139, 29, 39 and/or 49 described herebelow.

Catheter 100 and/or catheter 40 can be constructed and arranged to treat target tissue of a patient. In some embodiments, catheter 100 and/or catheter 40 is of similar construction and arrangement as catheter 100 of FIG. 1 described hereabove. Catheter 100 comprises handle 102 which attaches to a proximal end of shaft 110 and includes connector 103 for operable attachment to console 200. Positioned on the distal end or on a distal portion of shaft 110 is functional assembly 130. Catheter 40 comprises handle 42 which attaches to a proximal end of shaft 41 and includes connector 43 for operable attachment to console 200. Positioned on the distal end or on a distal portion of shaft 41 is functional assembly 45. Functional assembly 130 or 45 can comprise an expandable element selected from the group consisting of: an inflatable balloon such as balloons 136 and 46 shown; a radially expandable cage or stent; one or more radially deployable arms; an expandable helix; an unfurlable compacted coiled structure; an unfurlable sheet; an unfoldable compacted structure; and combinations of two or more of these. Functional assembly 130 or 45 can comprise an energy delivery element or other tissue treatment element, elements 135 and 135′, respectively, such as an energy delivery element configured to deliver thermal, electrical, light, sound and/or ablative chemical energy to target tissue. In some embodiments, treatment element 135 or 135′ comprises a mechanical abrader configured to treat tissue through abrasion. In some embodiments, functional assembly 130 or 45 comprises a balloon, balloon 136 and 46, respectively, which can be configured to receive one or more expansion and/or ablative fluids. Balloon 136 or 46 can comprise a compliant balloon, a non-compliant balloon, a pressure-thresholded balloon and/or otherwise be constructed and arranged as described in detail hereabove. Functional assembly 130 or 45 can be configured to both ablate (e.g. via a hot or cold ablative fluid) and neutralize the ablation (e.g. via a cooling or warming fluid, respectively), prior to and/or after the ablation, as described herein.

Via connectors 103 or 43, console 200 can provide and/or extract one or more fluids to and/or from one or more lumens or other flow pathways of catheters 100 or 40, such as fluid provided by reservoir 220 and/or propelled by (i.e. delivered and/or extracted by) pumping assembly 225. Console 200, via EDU 260, can be configured to provide energy to one or more treatment elements 135 or 135′ of catheters 100 or 40, respectively, such as energy contained in fluid at an ablative temperature (hot and/or cold), electrical energy (e.g. RF or microwave energy), light energy (e.g. laser light energy), or sound energy (e.g. subsonic or ultrasonic sound energy). In some embodiments, console 200 provides a fluid configured to treat target tissue with direct contact, such as an ablating agent (e.g. a sclerosant or other chemically ablative agent) and/or a fluid at an ablative temperature, either or both delivered directly to a target tissue surface.

In some embodiments, treatment elements 135 or 135′ comprises a fluid at an ablative temperature provided by console 200. In these embodiments, treatment elements 135 or 135′ can comprise a sufficiently hot fluid that is introduced into balloon 136 or 46, respectively, for a first time period to ablate target tissue, after which a cooling fluid is introduced into the balloon for a second time period, to extract heat from tissue (e.g. extract heat from target tissue and/or non-target tissue to reduce the ablation effect). Alternatively or additionally, a cooling fluid can be introduced into balloon 136 or 46 prior to the delivery of the hot fluid (e.g. for a third time period). In some embodiments, treatment element 135 or 135′ comprises a sufficiently cold fluid that is introduced into balloon 136 or 46, respectively, for a first time period to ablate target tissue, after which a higher temperature fluid is introduced into the balloon for a second time period, to warm tissue (e.g. warm target tissue and/or non-target tissue to reduce the ablation effect). Alternatively or additionally, a warming fluid can be introduced into balloon 136 or 46 prior to the delivery of the cold fluid (e.g. for a third time period). Both the ablative and ablation-reducing fluids can be provided by console 200. These fluids can be provided in a recirculating manner as described in applicant's co-pending U.S. patent application Ser. No. 16/438,362 (Attorney Docket No. 41714-704.302; Client Docket No. MCT-002-US-CON1), entitled “Heat Ablation Systems, Devices and Methods for the Treatment of Tissue”, filed Jun. 11, 2019, the content of which is incorporated herein by reference in its entirety for all purposes. Alternatively or additionally, these fluids can be provided in a single bolus manner as described in applicant's co-pending U.S. patent application Ser. No. 14/917,243 (Attorney Docket No. 41714-710.301; Client Docket No. MCT-023-US), entitled “Systems, Methods and Devices for Treatment of Target Tissue”, filed Mar. 7, 2016, the content of which is incorporated herein by reference in its entirety for all purposes. In some embodiments, thermal ablation is performed using system 10 as described herein.

In some embodiments, target tissue and/or tissue proximate the target tissue is cooled, heated and subsequently cooled again, such as via a procedure using catheter 100 and/or 40 of FIG. 2. In these embodiments, target tissue and/or tissue proximate the target tissue can be cooled during at least a portion of a first step, such as a first step including supplying a first fluid (e.g. a recirculating fluid) to functional assembly 130 or 45 for a first time period (e.g. a duration of at least 10 seconds or approximately between 15-30 seconds), wherein the first fluid is supplied at a cooling temperature (e.g. continuously supplied by reservoir 220 at a temperature of approximately 10° C.-25° C.). In a subsequent second step, target tissue and/or tissue proximate the target tissue can be heated (e.g. ablated) during at least a portion of the second step, such as a second step including supplying a second fluid (e.g. a recirculating fluid) to functional assembly 130 or 45 for a second time period (e.g. a duration of at least 5 seconds or approximately between 8-15 seconds), wherein the second fluid is supplied at a heat ablating temperature (e.g. continuously supplied by reservoir 220 at a temperature of approximately 85° C.-95° C.). In a subsequent third step, target tissue and/or tissue proximate the target tissue can be cooled during at least a portion of the third step, such as a third step including supplying a third fluid (e.g. a recirculating fluid) to functional assembly 130 or 45 for a third time period (e.g. a duration of at least 10 seconds or approximately between 15-30 seconds), wherein the second fluid is supplied at a cooling temperature (e.g. continuously supplied by reservoir 220 at a temperature of approximately 10° C.-25° C.). In some embodiments, other temperatures and/or durations for each heating or cooling cycle are used. In some embodiments, the second time period in which a hot fluid is supplied to functional assembly 130 or 45 comprises a time less than the first time period and/or the third time period. In some embodiments, the temperature of the fluid supplied to functional assembly 130 or 45 during the first time period and/or the third time period is at least 18° C. less and/or at least 60° C. less than the temperature of the fluid supplied to functional assembly 130 or 45 during the second time period. In some embodiments, the first temperature and the third temperature comprise a similar temperature. In some embodiments, a cooling fluid at approximately 10° C. is delivered to functional assembly 130 or 45 for approximately 30 seconds, after which an ablative fluid at approximately 95° C. is delivered to functional assembly 130 or 45 for approximately 12 seconds, after which a cooling fluid at approximately 10° C. is delivered to functional assembly 130 or 45 for approximately 30 seconds. Alternatively, a warming fluid can be delivered to functional assembly 130 or 45 prior to and/or after the delivery of a cryogenically ablative fluid (e.g. for the similar time periods as described herein in reference to heat ablation). In some embodiments, the volume, temperature and/or duration of fluid delivered to functional assembly 130 or 45 is automatically and/or dynamically adjusted, such as an adjustment performed based on a signal provided by one or more sensors as described herein. For example, a temperature and/or duration can be adjusted during a first ablation of an axial segment of intestine and/or during a subsequent second ablation of the same or different axial segment of intestine. In some embodiments, a pre-cooling and/or post-cooling step is used to avoid the need for a tissue expansion step (e.g. tissue expansion proximate tissue to be ablated in a heat ablation step). In other embodiments, a tissue expansion step is included.

In some embodiments, a first axial segment of tubular tissue is cooled (e.g. non-ablatively cooled), via functional assembly 130 or 45, for a first time period TP₁, and subsequently heat ablated for a second time period TP₂. A first reservoir 220_{A} includes the cooling fluid at a temperature T_{A}, (e.g. fluid continuously maintained or at least initially provided at temperature T_{A}) and a second reservoir 220_{B} includes the (heat) ablative fluid at a temperature T_{B} (e.g. fluid continuously maintained or at least initially provided at temperature T_{B}). In some embodiments, after the heat ablation during time period TP₂, an additional tissue cooling step is performed via functional assembly 130 or 45, for a third time period TP₃. Additionally, axial segments of tubular tissue can subsequently be treated (e.g. additional axial segments treated via tissue cooling and subsequent heat ablation, with or without a subsequent tissue cooling step). T_{A} can comprise a temperature at or below approximately 25° C., such as a temperature at or below approximately 20° C. and/or 15° C., and T_{B} can comprise a temperature at or above approximately 65° C., such as a temperature at or above approximately 75° C., 85° C. and/or 95° C. TP₁ can comprise a time duration of between 3 seconds and 60 seconds (e.g. between 20 seconds and 40 seconds); TP₂ can comprise a time duration of between 1 seconds and 30 seconds (e.g. between 5 seconds and 15 seconds); and TP₃ can comprise a time duration of between 3 seconds and 60 seconds (e.g. between 20 seconds and 40 seconds). In these embodiments, T_{A}, T_{B}, TP₁, TP₂ and/or TP₃ can be varied (e.g. automatically by system 10), based on information recorded by a sensor of the present inventive concepts (e.g. a sensor measuring temperature, pressure, flow rate and/or other parameter at one or more locations of catheter 100 and/or 40, console 200 or other component of system 10). One or more of T_{A}, T_{B}, TP₁, TP₂ and/or TP₃ can be held relatively constant or unchanged, during one or more axial tissue segment ablations. However, one or more of T_{A}, T_{B}, TP₁, TP₂ and/or TP₃ can vary (e.g. be allowed to vary), such as when T_{A} increases during an extraction of cooling fluid from catheter 100 (e.g. the recovered fluid warms the cooling fluid in the first reservoir 220_{A}). These variations (e.g. as measured by one or more sensors of system 10) can result in an adjustment (e.g. an automatic adjustment) to another parameter (e.g. T_{A}, T_{B}, TP₁, TP₂ and/or TP₃), such as an adjustment made by algorithm 251 (e.g. an algorithm comprising a lookup table including reservoir temperatures and corresponding treatment durations) based on a signal produced by one or more functional elements 109, 119, 139, 209, 229 and/or 309 described hereabove in reference to FIG. 1, that have been configured as a sensor (e.g. configured to provide a signal used to adjust one or more console settings 201). In some embodiments, T_{A}, T_{B} TP₁, TP₂ and/or TP₃ are varied based on the value of T_{A} and/or T_{B}. For example, if the temperature T_{A} of the cooling fluid were to increase during a multi-ablation procedure, the time period TP₂ and/or temperature T_{B} could be compensatingly adjusted (e.g. decreased). In some embodiments, time period TP₂ is decreased by up to 2 seconds (e.g. from an initial time period of approximately 11 to 13 seconds, in one or more decrements), as the temperature T_{A} increases by up to 16° C. (e.g. from a starting temperature of approximately 9° C.), such as during a clinical procedure comprising ablation of two or more axial segments (e.g. ablation of between two and six axial segments). While the previous embodiments have been described in reference to a cooling of tissue followed by a heat ablation of tissue (which may also include a subsequent tissue cooling step), alternatively, system 10 can be configured to (non-ablatively) warm tissue, followed by cryogenic ablation of tissue (which can also include a subsequent tissue warming step).

In some embodiments, treatment element 135 or 135′ comprises one or more energy or other tissue treatment elements positioned in, on and/or within functional assembly 130 or 45, respectively. Treatment element 135 or 135′ can comprise one or more energy delivery elements configured to deliver energy to target tissue, such as an energy delivery element selected from the group consisting of: a fixed or recirculating volume of fluid at a high enough temperature to ablate tissue; a fixed or recirculating volume of fluid at a low enough temperature to ablate tissue; one or more thermal energy delivery elements such as one or more elements configured to deliver heat energy or cryogenic energy; an array of electrodes such as an array of electrodes configured to deliver radiofrequency (RF) energy; one or more electromagnetic energy delivery elements such as one or more elements configured to deliver microwave energy; one or more optical elements configured to deliver light energy such as laser light energy; one or more sound energy delivery elements such as one or more elements configured to deliver subsonic and/or ultrasonic sound energy; one or more chemical or other agent delivery elements; and combinations of two or more of these. In some embodiments, catheter 100 or 40 is constructed and arranged to deliver RF energy, such as is described in applicant's co-pending U.S. patent application Ser. No. 16/711,236 (Attorney Docket No. 41714-706.302; Client Docket No. MCT-004-US-CON1), entitled “Electrical Energy Ablation Systems, Devices and Methods for the Treatment of Tissue”, filed Dec. 11, 2019; and/or to deliver ablative fluid directly to tissue, such as is described in applicant's co-pending U.S. patent application Ser. No. 14/609,334 (Attorney Docket No. 41714-707.301; Client Docket No. MCT-005-US), entitled “Ablation Systems, Devices and Methods for the Treatment of Tissue”, filed Jan. 29, 2015; the content of each of which is incorporated herein by reference in its entirety for all purposes.

In some embodiments, catheter 100 or 40 is further constructed and arranged to provide geometric information (e.g. diameter information) of a luminal structure such as the duodenum. In these embodiments, catheter 100 or 40, and associated functional assembly 130 or 45, respectively, can be of similar construction and arrangement as lumen diameter sizing catheter 30 and its functional assembly 35, described herein.

In some embodiments, system 10 comprises one or more devices for expanding target tissue or tissue proximate target tissue, such as tissue expansion catheter 20 or multi-function catheter 40. In some embodiments, target tissue to be treated comprises mucosal tissue and the tissue to be expanded comprises submucosal tissue proximate the mucosal tissue to be treated. In some embodiments, tissue expansion catheter 20 or multi-function catheter 40 is of similar construction and arrangement as catheter 100 described hereabove in reference to FIG. 1. In some embodiments, tissue expansion catheter 20 or multi-function catheter 40 is of similar construction and arrangement as a tissue expansion device described in applicant's co-pending U.S. patent application Ser. No. 15/274,948 (Attorney Docket No. 41714-712.301; Client Docket No. MCT-027-US), entitled “Injectate Delivery Devices, Systems and Methods”, filed Sep. 23, 2016, the content of which is incorporated herein by reference in its entirety for all purposes. Catheter 20 or 40 can be configured to expand a full or partial circumferential segment of luminal wall tissue, such as to expand one or more layers of submucosal tissue in one or more axial segments of the duodenum or other portion of the GI tract. Catheter 20 or 40 can be configured to expand multiple segments of GI tract tissue, such as multiple relatively contiguous segments of submucosal tissue expanded as described in detail herein.

Tissue expansion catheter 20 comprises handle 22 which attaches to a proximal end of shaft 21 and includes connector 23 for operable attachment to console 200. Positioned on the distal end of shaft 21 or on a distal portion of catheter 20 is functional assembly 25. Functional assembly 25 can comprise an expandable element selected from the group consisting of: an inflatable balloon such as balloon 26 shown; a radially expandable cage or stent; one or more radially deployable arms; an expandable helix; an unfurlable compacted coiled structure; an unfurlable sheet; an unfoldable compacted structure; and combinations of two or more of these.

Balloon 26 or 46 can comprise a compliant balloon, a non-compliant balloon, a pressure-thresholded balloon and/or otherwise it can be constructed and arranged as described in detail hereabove. Balloon 26 or 46 can comprise a tissue-contacting length of between 20 mm and 26 mm, such as a tissue-contacting length of approximately 23 mm. Balloon 26 can comprise a wall thickness of between 0.0002″ and 0.0010″, such as a wall thickness of approximately 0.0005″. Functional assembly 25 or 45 can be configured to expand to a diameter between 27.5 mm and 37.5 mm, such as a diameter of approximately 32.5 mm. Functional assembly 25 or 45 can be configured to be expanded via control 24 or 44, respectively, and/or via user interface 205 of console 200 (e.g. inflated and deflated by delivery and extraction, respectively, of air, water and/or other fluids by console 200).

Functional assembly 25 or 45 comprises one or more fluid delivery elements 28 or 48, respectively. The one or more fluid delivery elements 28 or 48 can each comprise an element selected from the group consisting of: needle such as a straight needle or a curved needle; nozzle; fluid jet; iontophoretic fluid delivery element; and combinations of two or more of these. The one or more fluid delivery elements 28 or 48 are configured to deliver injectate 221 and/or another fluid to tissue when functional assembly 25 or 45, respectively, is expanded (e.g. at least partially expanded with inflation fluid provided by console 200), positioning the fluid delivery elements 28 or 48 proximate (e.g. in contact with or close to) tissue to be expanded, such as luminal wall tissue of the GI tract.

The one or more fluid delivery elements 28 or 48 can be configured to be advanced (e.g. advanced into tissue) and retracted via control 24 of catheter 20 or control 44 of catheter 40, respectively. The one or more fluid delivery elements 28 or 48 can be positioned in one or more ports 27 or 47, respectively, as shown in FIG. 2. In some embodiments, a vacuum provided by console 200 causes tissue to tend toward and/or enter each port 27 or 47, such that each fluid delivery element 28 or 48, respectively, can inject fluid (e.g. injectate 221) into the engaged and/or captured tissue without having to extend significantly beyond the associated port 27 or 47 (e.g. each fluid delivery element can be configured to remain within the associated port during delivery of fluid into tissue captured within the port). By limiting excursion of fluid delivery element 28 or 48 out of port 27 or 47, respectively, risk of the fluid delivery element and/or injectate 221 penetrating through the outer surface of the GI tract is prevented or at least significantly reduced. In some embodiments, fluid can be delivered into tissue by fluid delivery element 28 or 48 with or without advancement of the fluid delivery element into the captured tissue (e.g. tissue is drawn into a port via an applied vacuum such that fluid delivery element penetrates or otherwise engages the tissue for fluid delivery without advancement of the fluid delivery element). In some embodiments, fluid delivery elements 28 or 48, ports 27 or 47, and/or other portions of tissue expansion catheter 20 or multi-function catheter 40, are of similar construction and arrangement as a tissue expansion device described in applicant's co-pending U.S. patent application Ser. No. 15/274,948 (Attorney Docket No. 41714-712.301; Client Docket No. MCT-027-US), entitled “Injectate Delivery Devices, Systems and Methods”, filed Sep. 23, 2016, the content of which is incorporated herein by reference in its entirety for all purposes.

In some embodiments, functional assembly 25 or 45 comprises three or more fluid delivery elements 28 or 48, respectively, which can be arranged in a circumferential pattern, such as three fluid delivery elements 28 or 48 arranged along a circumference and separated by approximately 120°. The multiple fluid delivery elements 28 or 48 can be configured to be advanced individually (e.g. via multiple controls 24 or 44 respectively), or simultaneously (e.g. via a single control 24 or 44). In some embodiments, two fluid delivery elements 28 or 48 are separated by approximately 180°. In some embodiments, four fluid delivery elements 28 or 48 are separated by approximately 90°.

In some embodiments, system 10 includes injectate 221 which can be provided by console 200 to catheter 20, and injectate 221 can be delivered into tissue by the one or more fluid delivery elements 28 or 48. Injectate 221 can comprise one or more materials as described hereabove in reference to injectate 221 of FIG. 1.

In some embodiments, catheter 20 or 40, and/or console 200 are configured to reduce a volume of fluid (e.g. liquid or gas) within functional assembly 25 or 45 (e.g. within balloon 26 or 46) as injectate 221 is delivered into tissue (e.g. submucosal tissue), such as to prevent excessive force being applied by functional assembly 25 or 45, respectively, to tissue proximate the expanding tissue (i.e. due to the decreasing luminal diameter proximate the expanding tissue in contact with functional assembly 25 or 45). In some embodiments, system 10 is constructed and arranged to inflate or otherwise expand functional assembly 25 or 45 (e.g. balloon 26 or 46) to a first target pressure, such as a pressure of approximately 0.7 psi. Injectate 221 is delivered via one or more fluid delivery elements 28 or 48 into submucosal tissue (e.g. simultaneously or sequentially). Fluid contained within functional assembly 25 or 45 (e.g. within balloon 26 or 46) can be reduced or increased to maintain the pressure at a second target pressure, for example a pressure higher than the first target pressure such as a pressure between 0.8 psi and 0.9 psi. Fluid of up to 10 ml can be injected while maintaining the second target pressure in functional assembly 25 or 45 (e.g. by decreasing the amount of fluid in the functional assembly to cause 1 mm steps of diameter decrease of the functional assembly).

In some embodiments, tissue expansion catheter 20 or multi-function catheter 40 is further constructed and arranged to provide geometric information (e.g. diameter information) of a luminal structure such as the duodenum or other intestinal location. In these embodiments, catheter 20 and functional assembly 25, or catheter 40 and functional assembly 45, can be of similar construction and arrangement as lumen diameter sizing catheter 30 and functional assembly 35, respectively, described herebelow.

In some embodiments, system 10 comprises one or more separate devices for estimating or otherwise measuring (e.g. “sizing”) the diameter, average diameter, equivalent diameter, minimum diameter, cross sectional area and/or other geometric measure (herein “diameter”) of luminal tissue, such as lumen diameter sizing catheter 30 or multi-function catheter 40. Sizing catheter 30 or multi-function catheter 40 can be constructed and arranged to be placed into one or more locations of the GI tract or other internal location of the patient and measure the diameter or other geometric parameter of tissue. In some embodiments, sizing catheter 30 or multi-function catheter 40 is of similar construction and arrangement as catheter 100 described hereabove in reference to FIG. 1. Catheter 30 or 40 can be configured to measure the diameter of multiple segments of intestinal or other GI tract tissue, such as to measure multiple diameters along the length of the duodenum.

Catheter 30 comprises handle 32 which attaches to a proximal end of shaft 31 and includes connector 33 for operable attachment to console 200. Positioned on the distal end of shaft 31 or on a distal portion of catheter 30 is functional assembly 35. Functional assembly 35 can comprise an expandable cage, balloon 36, or other expandable element as described herein, constructed and arranged to measure the inner surface diameter of tubular tissue (e.g. average diameter, equivalent diameter, minimum diameter, cross sectional area and/or other geometric measure of the inner surface of tubular tissue), such as a diameter of the duodenum or jejunum.

Balloon 36 or 46 can comprise a compliant balloon, a non-compliant balloon, a pressure-thresholded balloon and/or otherwise be constructed and arranged as described in detail hereabove. Functional assembly 35 or 45 can be configured to be expanded via control 34 or 44, respectively, and/or via user interface 205 of console 200 (e.g. inflated and deflated by delivery and extraction, respectively, of fluids by console 200).

Fluids delivered by console 200 to functional assembly 35 or 45 (e.g. fluids supplied by reservoir 220) can be provided at one or more predetermined pressures, or pressure profiles. Diameter measurements can be accomplished by performing a visualization procedure (manual or automated) that assesses functional assembly 35 or 45 diameter. Alternatively or additionally, functional assembly 35 or 45 can be controllably filled with a fluid, and controller 250 can include an algorithm (e.g. algorithm 251 described hereabove in reference to FIG. 1) that correlates the fluid volume and/or fluid pressure to the diameter of tubular tissue in contact with functional assembly 35 or 45. In some embodiments, subsequent selection (e.g. device model or size selection) and/or expansion diameter (e.g. inflated diameter chosen for sufficient apposition) of functional assemblies 130, 25 and/or 45 of catheters 100, 20 and/or 40, respectively, can be determined using the information provided by sizing catheter 30 and/or console 200. In some embodiments, catheter 30 or 40 performs one or more sizing procedures as described herein.

In some embodiments, functional assembly 35 or 45 comprises a balloon, expandable cage and/or other expandable element that includes two or more electrodes configured to provide a tissue impedance measurement whose value can be correlated to a level of apposition of functional assembly 35 or 45, respectively, and whose expanded diameter (e.g. visually or otherwise measured) correlates to a diameter of tubular tissue in contact with the expandable element. Alternatively or additionally, functional assembly 130 of catheter 100, functional assembly 25 of catheter 20 and/or functional assembly 45 of catheter 40 can be used to measure a diameter of the inner surface of tubular tissue, such as has been described hereabove in reference to functional assembly 35 and catheter 30.

In some embodiments, system 10 comprises one or more devices, such as multi-function catheter 40 shown, that are constructed and arranged to perform two or more functions selected from the group consisting of: treat target tissue such as to deliver energy or otherwise ablate target tissue; expand tissue such as to expand one or more layers of submucosal tissue (e.g. proximate to and/or including target tissue); and determine or estimate a diameter (e.g. an average diameter, equivalent diameter, minimum diameter, cross sectional area and/or other geometric measure) of a lumen of tubular tissue; and combinations of two or more of these. Multi-function catheter 40 is constructed and arranged to be placed into one or more locations of the GI tract or other internal location of the patient and perform two or more of the functions listed above. In some embodiments, multi-function catheter 40 is of similar construction and arrangement as catheter 100 described hereabove in reference to FIG. 1. Multi-function catheter 40 can be configured to perform the multiple functions at multiple segments of GI tract, such as multiple relatively contiguous axial segments of the duodenum or other intestinal location as is described herein.

Catheter 40 comprises handle 42 which attaches to a proximal end of shaft 41 and includes connector 43 for operable attachment to console 200. Positioned on the distal end of shaft 41 or on a distal portion of catheter 40 is functional assembly 45. Functional assembly 45 can comprise an expandable cage, a balloon (e.g. balloon 46 shown), and/or other expandable element constructed and arranged to be positioned in apposition with and/or in close proximity to the inner wall of tubular tissue, such as tissue of the duodenum, jejunum and/or other intestinal location. Balloon 46 can comprise a compliant balloon, a non-compliant balloon, a pressure-thresholded balloon and/or otherwise be constructed and arranged as described in detail hereabove. Functional assembly 45 can be configured to be expanded via control 44 and/or via user interface 205 of console 200 (e.g. inflated and deflated by delivery and extraction, respectively, of fluids by console 200).

Functional assembly 45 can comprise treatment element 135′, which can comprise a fluid at an ablative temperature delivered into functional assembly 45 by console 200 and/or an energy delivery element permanently positioned on, in and/or within functional assembly 45 (e.g. an energy delivery element configured to deliver thermal energy, electrical energy, light energy, sound energy and/or chemical energy as described herein). In some embodiments, treatment element 135′ comprises a mechanical abrader configured to treat tissue through abrasion. In some embodiments, treatment element 135′ is of similar construction and arrangement as functional element 139a of catheter 100 of FIG. 1 and/or treatment element 135 of catheter 100 of FIG. 2. Functional assembly 45 can be configured to both ablate (e.g. via a hot or cold ablative fluid) and neutralize (e.g. via a cooling or warming fluid, respectively), prior to and/or after the ablation, as described herein.

Alternatively or additionally, functional assembly 45 can comprise one or more elements configured to expand tissue, such as fluid delivery elements 48. Fluid delivery elements 48 can each be positioned within one or more ports 47 as shown. Fluid delivery elements 48 and ports 47 can be constructed and arranged as described hereabove in reference to fluid delivery element 139c and ports 137, respectively, of catheter 100 of FIG. 1.

Catheters 100, 20, 30 and/or 40 can comprise one or more functional elements, such as functional elements 139, 29, 39 and/or 49, respectively, shown positioned in, on and/or within functional assemblies 130, 25, 35 and 45, respectively. Alternatively or additionally, one or more functional elements 139, 29, 39 and/or 49 can be located at a different location of the associated device, such as in, on and/or within the associated shaft and/or handle of the device. In some embodiments, one or more functional elements 139, 29, 39 and/or 49 comprise a sensor, such as a sensor selected from the group consisting of: physiologic sensor; blood glucose sensor; blood gas sensor; blood sensor; respiration sensor; EKG sensor; EEG sensor; neuronal activity sensor; blood pressure sensor; flow sensor such as a flow rate sensor; volume sensor; pressure sensor; force sensor; sound sensor such as an ultrasound sensor; electromagnetic sensor such as an electromagnetic field sensor or an electrode; gas bubble detector such as an ultrasonic gas bubble detector; strain gauge; magnetic sensor; ultrasonic sensor; optical sensor such as a light sensor; chemical sensor; visual sensor such as a camera; temperature sensor such as a thermocouple, thermistor, resistance temperature detector or optical temperature sensor; impedance sensor such as a tissue impedance sensor; and combinations of two or more of these. Alternatively or additionally, one or more functional elements 139, 29, 39 and/or 49 comprise a transducer, such as a transducer selected from the group consisting of: an energy converting transducer; a heating element; a cooling element such as a Peltier cooling element; a drug delivery element such as an iontophoretic drug delivery element; a magnetic transducer; a magnetic field generator; an ultrasound wave generator such as a piezo crystal; a light producing element such as a visible and/or infrared light emitting diode; a motor; a pressure transducer; a vibrational transducer; a solenoid; a fluid agitating element; and combinations of two or more of these. Functional elements 139, 29, 39 and/or 49 can be electrically connected to EDU 260 (e.g. to receive power, send signals and/or receive signals), such as via an electrical connection provided by connector 203. Functional elements 139, 29, 39 and/or 49 can send or receive signals from controller 250 of console 200, such as one or more sensor signals used to control ablation energy provided by console 200. Functional elements 139, 29, 39 and/or 49 can be activated and/or otherwise controlled via controls 104, 24, 34 and/or 44, respectively. Alternatively or additionally, user interface 205 of console 200 can be configured to allow operator control of functional elements 139, 29, 39 and/or 49.

In some embodiments, console 200 comprises one or more functional elements 209, comprising a sensor or transducer as described hereabove. Functional element 209 can comprise one or more pressure sensors, such as one or more pressure sensors configured to provide a signal used to regulate fluid delivery provided to one or more of catheters 100, 20, 30 and/or 40. Functional element 209 can comprise one or more temperature sensors, such as one or more temperature sensors that provide a signal used to regulate temperature of one or more fluids of console 200. Functional element 209 can be positioned to measure a parameter (e.g. temperature or pressure) of fluid within reservoir 220, within pumping assembly 225 and/or within a fluid conduit of console 200.

In some embodiments, system 10 comprises one or more agents configured to be delivered to the patient, such as agent 420. Agent 420 can be delivered by one or more of catheters 100, 20, 30, 40 and/or 50, or by a separate device such as a syringe or other medication delivery device. In some embodiments, injectate 221 comprises agent 420, such as when agent 420 is delivered by one or more fluid delivery elements 139c as described herein. In some embodiments, agent 420 comprises an anti-peristaltic agent, such as L-menthol (i.e. oil of peppermint). Alternatively or additionally, agent 420 can comprise glucagon, buscopan, hyoscine, somatostatin, an opioid agent, and/or any anti-peristaltic agent. Agent 420 can be delivered into the GI tract, such as via endoscope 50a, sheath 80 and/or catheters 100, 20, 30 and/or 40. Agent 420 can be delivered systemically, such as via an intravenous or intra-arterial access line, or injected directly into tissue. Agent 420 can comprise a drug or other agent as described hereabove in reference to agent 420 of FIG. 1.

As described above, user interface 205 can comprise safety-switch 206 such as a foot-activated switch. Safety-switch 206 can be configured to allow a clinician to activate or modify one or more processes of system 10 without having to use his or her hands (e.g. without having to use a digit of the hand). In some embodiments, system 10 is constructed and arranged to perform a function selected from the group consisting of: automatic contraction (e.g. deflation) of a functional assembly if safety-switch 206 is not activated (e.g. continuously or semi-continuously pushed, pressed or otherwise activated, such as by a foot or digit of an operator); automatic replacement of ablative fluid (e.g. hot fluid) with neutralizing fluid (e.g. cold fluid) if safety-switch 206 is not activated; initiate introduction of ablative fluid (e.g. hot fluid) into a functional assembly by activation of safety-switch 206 (e.g. after a functional assembly has been pre-expanded with cold fluid and an operator has confirmed proper position for treatment); allow hands-free activation (e.g. initiation) of a treatment step such that one or more operators can maintain their hands on one or more of endoscope 50a and/or catheters 100, 20, 30 and/or 40; allow hands-free activation (e.g. initiation) of a treatment step such that the required number of operators is reduced; and combinations of two or more of these.

Each of catheters 100, 20, 30 and/or 40 can be provided in one or more sizes, such as one or more lengths of the associated shaft 110, 21, 31 and/or 41, respectively, and/or one or more diameters (e.g. expanded diameter) of the associated functional assembly 130, 25, 35 and/or 45, respectively. Luminal sizing as described herein, and/or other anatomical information, can be used to select the appropriately sized device to treat the patient.

In some embodiments, system 10 of FIG. 2 is configured to perform a medical procedure on a patient as described herebelow in reference to FIG. 7.

Applicant has conducted human studies with the systems, methods and devices of the present inventive concepts. Included below are results of early human clinical studies conducted by the applicant, and associated data collected.

Some patients received treatment of approximately 9 cm of relatively full-circumferential axial length of duodenal mucosa (via three approximately 3 cm hot fluid balloon-based ablations), and some patients received treatment of less than or equal to 6 cm of relatively full-circumferential axial length of duodenal mucosa (via two or less approximately 3 cm hot fluid balloon-based ablations).

Early results showed: baseline HbA1c was 9.2% and FPG was 187 mg/dl. One month post-procedure, HbA1c was reduced by 1.1% in LS-DMR patients (patients receiving duodenal mucosa treatments of approximately 9 cm (e.g. 9.3 cm) of duodenal tissue) but only 0.1% in SS-DMR patients (patients receiving duodenal mucosa treatment of approximately 3 cm (e.g. 3.4 cm) of duodenal tissue, the data representing 12 LS-DMR patients vs 7 SS-DMR patients, each group at one month (p=0.058). By three months, HbA1c was reduced by approximately 2% in LS-DMR patients but was unchanged in SS-DMR patients (N=5 in each group at three months). FPG reductions in LS-DMR patients were −64 mg/dl and −67 mg/dl at one and three months.

Table A below shows a breakdown of a number of patients who received various quantities of duodenal axial segment treatments comprising delivery of heat from an ablative fluid delivered to a balloon-based treatment assembly. In this study, 35 patients were treated in a dosimetric evaluation of the systems, methods and devices described herein. An ablation is defined as an axial length of circumferentially ablated tissue, ablated with a single positioning of the balloon and subsequent hot fluid delivery to the balloon. Ablation dose is defined as the total length of circumferentially ablated tissue on a single procedural day. A single patient received 5 ablations (the highest dose administered), and duodenal stenosis presented as food intolerance and epigastric discomfort. After endoscopic balloon dilation, the patient recovered without further issue. This patient with the duodenal stenosis lost a substantial amount of weight in the two weeks after the development of stenosis (nearly 10 kilograms). Controlled duodenal stenosis may be an effective means of achieving substantial weight loss with its attendant benefits on metabolic or obesity-related ailments. Creation of a therapeutic restriction can be performed as described in applicant's co-pending U.S. patent application Ser. No. 16/267,771 (Attorney Docket No. 41714-711.302; Client Docket No. MCT-024-US-CON1) entitled “Systems, Devices and Methods for the Creation of a Therapeutic Restriction in the Gastrointestinal Tract”, filed Feb. 5, 2019, the content of which is incorporated herein by reference in its entirety for all purposes.

TABLE A

| Number of Duodenal | Number |
|---|---|
| Ablations | of Patients |
| 0 | 2 |
| 1 | 6 |
| 2 | 4 |
| 3 | 22 |
| 4 | 0 |
| 5 | 1 |

In some embodiments, the systems, devices and methods of the present inventive concepts can be configured to deliver at least two ablations to target tissue (e.g. at least two sequential deliveries of energy or other treatments to different axial segments of GI mucosa), such as to deliver at least three ablations to target tissue. In some embodiments, a minimum and/or maximum amount of duodenal mucosa is treated, such as has been described hereabove.

Table B is a table of cumulative demographic information for the first 21 patients of the applicant's studies. These baseline characteristics are generalizable and relevant to the Type 2 diabetes population.

TABLE B

| Characteristic | Value (N = 32) | N in calc |
|---|---|---|
| Duration diabetes-yr | 5.1 +/− 2.9 | 27 |
| Age-yr | 52.9 +/− 7.6 | 26 |
| Female sex-N (%) | 12 (46.2) | 26 |
| Weight-kg | 86.7 +/− 13.2 | 26 |
| Height-cm | 165.7 +/− 10.2 | 26 |
| BMI-kg/m² | 31.6 +/− 4.0 | 26 |
| BP Systolic-mmHg | 122.5 +/− 16.2 | 26 |
| BP Diastolic-mmHg | 77.2 +/− 8.0 | 26 |
| Medications-N | 1.7 +/− 0.6 | 19 |

In some embodiments, the systems, devices and methods of the present inventive concepts can be configured to treat patients with a characteristic selected from the group consisting of: duration of diabetes less than 10 years; age between 18 yrs and 75 yrs; BMI between 20 and 60, such as a BMI between 24 and 40; and combinations thereof.

Table C is a table of results of applicant's studies, detailing recorded dose dependent improvements in glycemic control. Applicant measured three validated measures of glycemic control: Hemoglobin A1c (HbA1c), fasting plasma glucose (FPG), and two hour post-prandial glucose (2hPG).

TABLE C

| | | Baseline | | 1 month | | | 3 months | | |
|---|---|---|---|---|---|---|---|---|---|
| | | N | Value | N | Value | Delta | N | Value | Delta |
| HbA1c | All subjects | 26 | 9.22 | 23 | 8.25 | −0.97 | 14 | 7.99 | −1.23 |
| | ≥3 ablations | 16 | 9.42 | 15 | 7.91 | −1.51 | 8 | 7.08 | −2.34 |
| | <3 ablations | 10 | 8.91 | 8 | 8.90 | −0.01 | 6 | 9.22 | 0.31 |
| FPG | All subjects | 26 | 187.6 | 23 | 141.7 | −45.8 | 14 | 160.1 | −27.4 |
| | ≥3 ablations | 16 | 186.7 | 15 | 123.1 | −63.6 | 8 | 129.8 | −56.9 |
| | <3 ablations | 10 | 189.0 | 8 | 176.6 | −12.4 | 6 | 200.7 | 11.7 |
| 2hPG | All subjects | 26 | 263.1 | 20 | 199.3 | −63.9 | 14 | 207.1 | −56.0 |
| | ≥3 ablations | 16 | 268.9 | 13 | 183.6 | −85.3 | 8 | 163.8 | −105.1 |
| | <3 ablations | 10 | 253.9 | 7 | 228.3 | −25.6 | 6 | 264.8 | 10.9 |

In some embodiments, the systems, devices and methods of the present inventive concepts can be configured to provide a therapeutic benefit selected from the group consisting of: a reduction in HbA1c of at least 0.7%, 1.0% or 1.5% at three months, such as a reduction of approximately 2.18% at three months; an FPG of no more than 150 mg/dl, 126 mg/dl or 100 mg/dl, such as an FPG that can result with a reduction of approximately 63.5 mg/dl; a 2hPG of no more than 250, 200 or 175, such as an 2hPG that can result with a reduction of approximately 103.7; and combinations thereof.

In some embodiments, an absolute change of at least 0.7%, 1.0%, 1.5% and/or 2.0% in HbA1c is expected. In some embodiments, a relative change above an HbA1c target is expected, such as a relative change of at least 50%, 75% or 100%, such as when the target HbA1c is an HbA1c of approximately 6.5%, 7.0% or 7.5%. It has been reported that a 1% absolute change in HbA1c correlates to a 40% reduction in risk of microvascular complication due to diabetes. A 0.5% change is considered clinically significant and likely to result in therapeutic benefit.

Applicant's human clinical studies have shown various therapeutic benefits of the systems and methods of the present inventive concepts, as is described in applicant's co-pending U.S. patent application Ser. No. 16/400,491 (Attorney Docket No. 41714-716.301; Client Docket No. MCT-035-US), entitled “Systems, Device, and Methods for Performing Medical Procedures in the Intestine”, filed May 1, 2019, the content of which is incorporated herein by reference in its entirety for all purposes.

Studies have shown that an approximately 2% HbA1c reduction is achieved in patients receiving three or more ablations (via a balloon-based treatment element with a treatment length of at least 20 mm, correlating to at least 60 mm of cumulating duodenal length treated) compared with no change in those receiving fewer than 3 ablations (e.g. when less than 40 mm of cumulative duodenal length treated). These studies have also demonstrated: a reduction in FPG levels (which remain stable between one and three months post procedure); an improvement in 2hPG measurements; a durable reduction in HbA1c percentages at 120 days post-treatment in four out of five patients; fasting insulin change data, over three months, showing an improvement in the health of the beta cell; SF-36 Mental value changes, showing improved patient satisfaction through better glycemic control; weight change in study patients, showing that weight loss was also noticed in a dose dependent manner; data suggesting that weight loss and HbA1c are not well correlated based on 30 day post treatment data;

In some embodiments, the systems, devices and methods of the present inventive concepts can be configured to achieve an HbA1c level at or below 7.5%, or 7.0% or 6.5%, such as at a time period of three months or more, such as by ablating a cumulative length of duodenal mucosa greater than 6 cm, greater than 7 cm, greater than 8 cm or greater than 9 cm (e.g. via 2, 3 or more ablations as described herein). In some embodiments, the systems, devices and methods of the present inventive concepts can be configured to maintain HbA1c below 7.5% at 150 days. Note that three out of four patients are also on lower levels of medications than were being administered prior to the tissue treatment procedure.

In some embodiments, the systems, devices and methods of the present inventive concepts can be configured to cause an improvement in a patient condition as measured by the clinical standard SF-36 Health Survey, such as an improvement in the SF-36 Mental Change score of at least 3 points, at least 5 points or at least 10 points.

In some embodiments, the systems, devices and methods of the present inventive concepts can be configured to achieve at least 3 kg or at least 4 kg of weight loss.

Table D is a table presenting the large effect size of high dose cohort being statistically significantly better than low dose cohort.

TABLE D

| 1 MONTH | | | |
|---|---|---|---|
| Characteristic | 3 or more | less than 3 | p-value |
| Number subjects | 15 | 8 | |
| Baseline HbA1c-% | 9.39 +/− 1.42 | 9.08 +/− 1.03 | 0.58 |
| HbA1c Change-% | −1.49 +/− 0.92 | −0.18 +/− 1.00 | 0.0047 |
| Baseline FPG-mg/dL | 187 +/− 68 | 202 +/− 45 | 0.61 |
| FPG Change-mg/dL | −64 +/− 74 | −25 +/− 44 | 0.19 |
| 3 MONTH | | | |
| Characteristic | 3 or more | less than 3 | p-value |
| Number subjects | 8 | 6 | |
| Baseline HbA1c-% | 9.36 +/− 1.48 | 9.30 +/− 1.11 | 0.93 |
| HbA1c Change-% | −2.29 +/− 1.24 | −0.08 +/− 1.61 | 0.013 |
| Baseline FPG-mg/dL | 187 +/− 55 | 218 +/− 33 | 0.25 |
| FPG Change-mg/dL | −57 +/− 46 | −18 +/− 64 | 0.20 |

Human studies using the systems, devices and methods of the present inventive concepts have demonstrated significant effectiveness, such as at least a 2% HbA1c reduction in numerous patients at three months, a strong indication of clinical value for patients with poorly controlled glucose levels. The studies demonstrated excellent concordance between HbA1c and other surrogate markers such as fasting glucose and post-prandial glucose. The studies also demonstrated clinically meaningful weight loss. In some embodiments, the systems, devices and methods of the present inventive concepts can be used to treat naïve patients with an HbA1c of more than 6%, 6.5%, or 7%. The treatment could further include the administration of metformin (e.g. an agent 420 comprises metformin). The treatment of the present inventive concepts (with or without the administration of metformin or other single drug) could provide a therapeutic benefit to the patient better than a treatment comprising drug therapy alone (e.g. metformin and/or another single drug therapy). In some embodiments, agent 420 can comprise metformin and a second-line drug that are included in the treatment of the present inventive concepts. Treatment outcomes would include improvement in HbA1c, such as patients who achieve an improvement (i.e. reduction) of at least 1% in HbA1c and/or patients who achieve a target HbA1c of less than or equal to 6.0%, 6.5%, 7.0%, or 7.5%. Treatment can also include reduction in hypoglycemic events, improved quality of life, weight loss, and combinations of the above.

Included below are results of continued studies and associated data collected through Jul. 8, 2015.

Applicant's continued studies included the recording of various patient parameters affected by the treatment of the present inventive concepts, these parameters including but not limited to: HbA1c, fasting blood glucose and post prandial glucose. Patients received between one and five ablations (e.g. two to five sequential ablations performed along two to five axial segments of the duodenum distal to the ampulla of Vater) on a single procedural day. The ablations were delivered by an expandable balloon filled with hot fluid at an ablative temperature, as described in detail herein. The data below in Table E were collected from 39 patients with the following patient demographics:

TABLE E

| Characteristic | Value (N = 39) |
|---|---|
| Duration diabetes-yr | 5.9 +/− 2.2 |
| Age-yr | 53.7 +/− 7.3 |
| Female sex-N (%) | 14 (35.9) |
| Weight-kg | 85.1 +/− 12.0 |
| Height-cm | 165.5 +/− 8.8 |
| BMI-kg/m² | 31.0 +/− 3.4 |

Procedures were completed using general anesthesia. All patients were discharged on either the day of procedure (19/39) or after an overnight stay (20/39).

The treatment of the present inventive concepts may offer an even more significant and durable clinical effect when coupled with intensive medical management. The treatment effect does not appear to be weight dependent. Patients did not report any food intolerance or change in food preference that might explain the HbA1c reduction. While patients lost a small amount of weight, the magnitude of weight loss is likely not enough to explain the degree of HbA1c improvement. Furthermore, there did not appear to be any correlation between the magnitude of HbA1c reduction and weight loss.

In some embodiments, the systems, devices and methods of the present inventive concepts can reduce the need for insulin therapy in a larger proportion of patients, such as to provide durable glycemic control with or without the therapies administered to the patient prior to the treatment of the present inventive concepts, or with a decrease in dosage of one or more previously administered medications.

The systems, devices and methods of the present inventive concepts can be configured to treat patients with microvascular disease or patients with a high risk of microvascular disease, such as to improve patient health and/or eliminate or otherwise reduce the need for one or more medications (e.g. one or more insulin medications). The treatment can be configured to reduce diabetic retinopathy (e.g. as shown in a reduction in diabetic retinopathy score), proteinuria and/or peripheral neuropathy severity. Additionally or alternatively, the treatment can be configured to reduce the effects of macrovascular disease such as myocardial infarction, stroke, peripheral vascular disease, CV death, and combinations of two or more of these.

The systems, devices and methods of the present inventive concepts can be configured to treat patients with a disease or disorder of the liver, such as non-alcoholic fatty liver disease (NAFLD) and/or non-alcoholic steatohepatitis (NASH). For example, treatment element 135 of catheter 100 and/or treatment element 135′ of catheter 40 can be configured to modify one or more axial segments of the intestine (e.g. ablate a full circumferential or partial circumferential axial segment of duodenal mucosal and/or submucosal tissue). In some embodiments, intestinal submucosal tissue of an axial segment of intestine is expanded (e.g. by catheter 20 or catheter 40 as described hereabove in reference to FIG. 2), prior to ablation of at least the mucosal layer relatively within the expanded submucosal tissue. In some embodiments, the mucosal tissue is ablated by introducing hot fluid into balloon 136 of catheter 100 or balloon 46 of catheter 40. In some embodiments, tissue treatment element 135 of catheter 100 or tissue treatment element 135′ of catheter 40 comprises an element selected from the group consisting of: an ablative fluid delivered to a balloon or other expandable fluid reservoir; a tissue treatment element comprising an energy delivery element mounted to an expandable assembly such as an electrode or other energy delivery element configured to deliver radiofrequency (RF) energy and/or microwave energy; a light delivery element configured to deliver laser or other light energy; a fluid delivery element (e.g. a sponge or a nozzle) configured to deliver ablative fluid directly onto tissue; a sound delivery element such as an ultrasonic and/or subsonic sound delivery element; and combinations thereof, as described in detail herein. In some embodiments, a patient with NAFLD/NASH is selected and treated as described herein.

Applicant's clinical studies described hereabove have demonstrated potential benefit to patients with a liver disease or disorder such as NAFLD/NASH, such as a NAFLD/NASH patient that is also afflicted with Type 2 diabetes. Studies have shown an improvement (reduction) in the level of liver transaminases found in the treated patients.

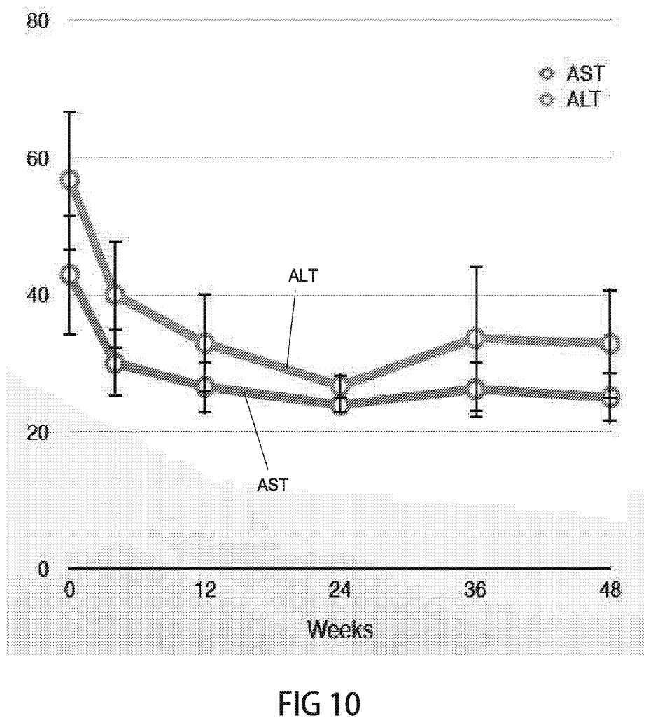

FIG. 10 represents an improvement (reduction) in the level (expressed in mg/dL) of two liver transaminases, aspartate transaminase (AST) and alanine transaminase (ALT), that resulted after a mucosal treatment of the present inventive concepts. The data presented in FIG. 10 represents 13 patients through week 24, and 8 (of the 13) patients through week 48. The data presented is representative of patients that had at least 3 cm of duodenal mucosa treated, such as when two or more axial segments of duodenal mucosa were treated to achieve a cumulative treated length of at least 6 cm or at least 9 cm. Pre-procedure, each patient had elevated baseline levels of AST and ALT as shown, which is indicative of inflammation of the liver. The AST and ALT levels were sustainably reduced after treatment of multiple segments of duodenal mucosa using the systems, devices and methods of the present inventive concepts. These reductions correlate to one or more of: improvement in steatosis, reduced inflammation of the liver and/or reduced fibrosis of the liver. In some embodiments, the methods of the present inventive concepts are configured to improve steatosis, reduce cirrhosis, reduce inflammation of the liver, reduce fibrosis of the liver and/or reduce liver failure.

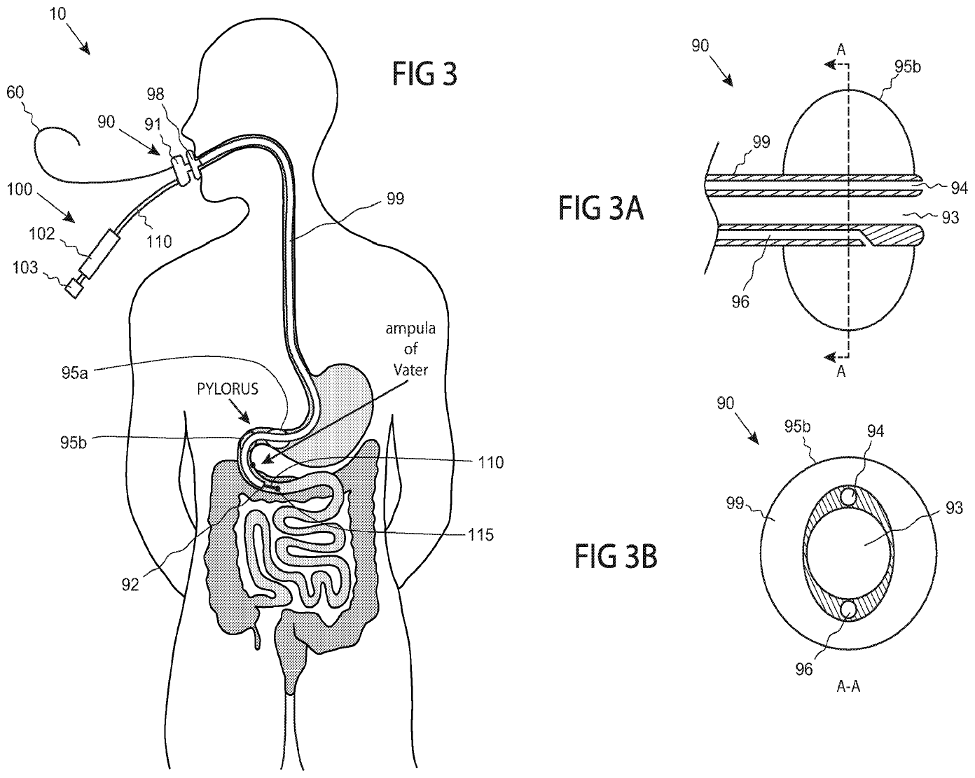

Referring now to FIG. 3, an anatomic view of a system for performing a medical procedure comprising a catheter and a sheath for inserting the catheter into the intestine of the patient is illustrated, consistent with the present inventive concepts. System 10 comprises catheter 100 which has been inserted through the mouth of the patient and advanced through the stomach to a location distal to the patient's pylorus. System 10 can further comprise introducer 90 (e.g. an introducer sheath), through which catheter 100 can be inserted as shown. System 10 can further comprise guidewire 60. System 10 can comprise one or more other components, such as console 200 and other components not shown, but of similar construction and arrangement to those described hereabove in reference to system 10 of FIG. 1 or system 10 of FIG. 2. Catheter 100 comprises connector 103, handle 102, shaft 110, tip 115, and other components, such as those described hereabove in reference to catheter 100 of FIG. 1, or catheters 100, 20, 30 and/or 40 of FIG. 2.

Introducer 90 comprises an elongate, flexible tube, shaft 99, and an input port 91 on the proximal end of shaft 99. Input port 91 can include a funnel-shaped or other opening configured to assist in the introduction of catheter 100 or other devices into a lumen of introducer 90. Input port 91, or another proximal portion of introducer 90, can be configured to attach introducer 90 to an endoscope or other body introduction device (e.g. device 50 described herein). In some embodiments, input port 91 comprises a strain relief configured to attach introducer 90 to a body introduction device. Bite block 98 can be positioned about shaft 99 at a location relatively proximate to input port 91. Positioned along a distal portion of shaft 99 are one or more anchor elements, such as anchor elements 95a and 95b shown. Anchor elements 95a and 95b can comprise a radially expandable structure, such as an expandable structure selected from the group consisting of: an inflatable balloon; a radially expandable cage or stent; one or more radially deployable arms; an expandable helix; an unfurlable compacted coiled structure; an unfurlable sheet; an unfoldable compacted structure; and combinations of two or more of these. Anchor elements 95a and 95b have been positioned at locations proximal and distal, respectively, to the pylorus, and subsequently radially expanded, such as to anchor distal end 92 of shaft 99 at a location distal to the ampulla of Vater (e.g. to avoid inadvertently treating or otherwise adversely affecting the ampulla of Vater and/or tissue proximate the ampulla of Vater). In some embodiments, anchor element 95a and/or 95b can be configured to be inflated within the duodenal bulb of the patient.

In some embodiments, shaft 99 comprises a variable stiffness along its length, such as a more flexible distal portion constructed and arranged to be positioned distal to the pylorus, than a portion that would be positioned proximal to the pylorus (e.g. to avoid a “slack” segment in the stomach when advancing catheter 100 through shaft 99). In some embodiments, shaft 99 comprises a shaft including a braided portion. In some embodiments, introducer 90 comprises a non-circular cross-section, such as to efficiently couple with an endoscope or other body introduction device (e.g. not shown but such as device 50 described herein), such as a non-circular cross-section selected from the group consisting of: oval; kidney shape; and combinations thereof.

FIGS. 3A and 3B illustrate side sectional and end sectional views, respectively, of the distal portion of introducer 90, without an inserted catheter 100 nor an inserted guidewire 60. FIG. 3B is a section along line A-A of FIG. 3A. Shaft 99 includes a lumen 94, such as a lumen constructed and arranged to slidingly receive a guidewire, such as guidewire 60, to permit over-the-wire advancement and retraction of introducer 90. Shaft 99 further includes working channel 93, such as a lumen constructed and arranged to slidingly receive a treatment or diagnostic device, such as catheter 100 as described herein. In some embodiments, working channel 93 comprises a diameter greater than or equal to 10 mm, or 20 mm. In some embodiments, introducer 90 is advanced to a desired location (e.g. with or without catheter 100 residing within working channel 93), and subsequently tip 115 of catheter 100 is advanced out of distal end 92 of introducer 90. Shaft 99 can further comprise a lumen 96, which can be configured as an inflation lumen when one or more of anchor elements 95a or 95b comprise a balloon or other inflatable structure. Alternatively, lumen 96 can be constructed and arranged to receive a translatable rod or other filament, such as when anchor element 95a and/or 95b comprise an expandable scaffold, radially deployable arm or other structure whose expansion and contraction is controlled by the translation of the filament. Working channel 93 and/or lumen 94 can be configured as a port for delivering and/or extracting fluids from the intestine (e.g. to insufflate and/or desufflate, respectively, a segment of the intestine).

In some embodiments, system 10 of FIG. 3 comprises one or more sensors, such as one or more functional elements 109, 119, 139, 209, 229 and/or 309 described hereabove in reference to FIG. 1, that have been configured as a sensor. These one or more sensors can be configured to provide a signal, such as a signal used to adjust one or more console 200 settings (e.g. console settings 201) of the present inventive concepts. In some embodiments, functional assembly 130 comprises one or more functional elements, such as functional element 139a, 139b and/or 139c described hereabove in reference to FIG. 1, such as a functional element constructed and arranged to perform a therapeutic and/or diagnostic medical procedure, as described herein.

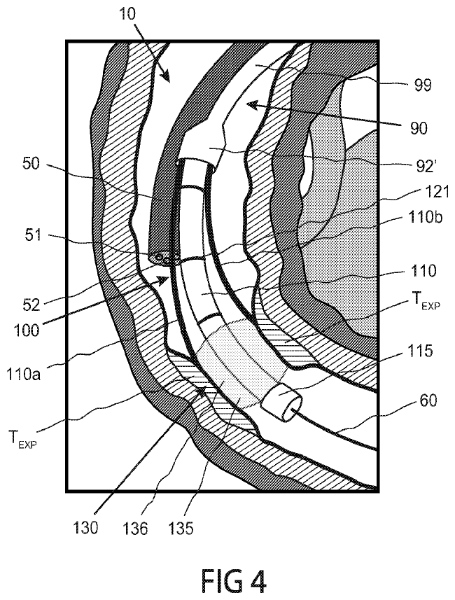

Referring now to FIG. 4, a sectional view of the distal portion of a system including an endoscope and a treatment device inserted into a duodenum of a patient is illustrated, consistent with the present inventive concepts. System 10 includes catheter 100, such as a catheter configured to both expand tissue (e.g. circumferentially expanded tissue T_{EXP} shown), as well as treat (e.g. ablate) target tissue. Catheter 100 and other components of system 10 can be of similar construction and arrangement to the similar components described hereabove in reference to FIGS. 1 and/or 2. Catheter 100 is shown positioned in a side-by-side arrangement with endoscope 50, which can include one or more working channels, lumen 51 shown, and a visible light and/or infrared camera, camera 52. Catheter 100 has been advanced over a guidewire 60 and through introducer 90 (e.g. to a location in the small intestine of the patient). Introducer 90 includes shaft 99 with expanded distal end 92′. Distal end 92′ can be sized to surround a bulbous distal end of catheter 100, such as tip 115 shown. In some embodiments, catheter 100 is advanced over guidewire 60 but not through introducer 90.

Catheter 100 includes a treatment assembly, functional assembly 130, which is shown in its expanded state, and positioned on a central shaft, shaft 110. Functional assembly 130 can include one or more fluid delivery elements, not shown but such as one or more (e.g. three) needles or other fluid delivery elements such as fluid delivery elements 139c described hereabove. The fluid delivery elements can be positioned in a circumferential arrangement (e.g. three needles positioned approximately 120° apart along functional assembly 130), each fluid delivery element fluidly attached to a fluid delivery tube, such as shafts 110a and 110b shown. The fluid delivery elements may be each be positioned in a port, such as port 137, also not shown but described hereabove, such that a vacuum can be applied to tissue to cause the tissue to be drawn into the port 137, after which fluid can be injected into the tissue via the associated fluid delivery element. Functional assembly 130 can comprise a radially expandable assembly, such as balloon 136, into which an ablative element 135 can be positioned (e.g. an electrode configured to deliver RF or other electromagnetic energy) and/or introduced (e.g. hot or cold ablative fluid introduced into balloon 136). Catheter 100 can comprise one or more visualizable markers, such as radiopaque or visible marker bands, circumferential marker 121 (3 shown). In some embodiments, a neutralizing fluid (e.g. a cooling or warming fluid) is introduced into balloon 136 prior to and/or after ablation of tissue.

In the embodiment shown in FIG. 4, tissue surrounding and proximate functional assembly 130 has been expanded (circumferentially expanded tissue T_{EXP} shown), such that ablation or other treatment can be performed by functional assembly 130 on the mucosal layer of the axial segment of the small intestine (e.g. the duodenum) proximate functional assembly 130 (e.g. proximate balloon 136), such as is described herein. After the tissue treatment is performed, functional assembly 130 can be radially compacted (e.g. balloon 136 at least partially deflated), translated (e.g. advanced or retracted to a neighboring or distant axial segment), after which similar tissue expansion (e.g. submucosal tissue expansion) and tissue treatment (e.g. mucosal tissue ablation) can be performed, such as to treat a patient medical condition (e.g. a disease and/or disorder) as described herein.

Referring now to FIGS. 5A and 5B, end and side views of the distal portion of a catheter including recessed ports and shaft-located vacuum port are illustrated, consistent with the present inventive concepts. Catheter 100 comprises shaft 110, functional assembly 130 (shown in its expanded state), and other components, such as one or more components of similar construction and arrangement to those described hereabove in reference to catheter 100 of FIG. 1 or FIG. 2, such as one or more conduits 111, some of which have been removed for illustrative clarity (three conduits 111 shown in FIG. 5B). In some embodiments, tip 115 comprises a bulbous tip positioned on the distal end of catheter 100 as shown. Functional assembly 130 is configured to radially expand and contract, and it can comprise an expandable element selected from the group consisting of: an inflatable balloon such as balloon 136 shown; a radially expandable cage or stent; one or more radially deployable arms; an expandable helix; an unfurlable compacted coiled structure; an unfurlable sheet; an unfoldable compacted structure; and combinations of two or more of these as described herein. Functional assembly 130 is shown in a radially expanded state in FIGS. 5A and 5B.

In some embodiments, functional assembly 130 includes one or more recesses, such as the three recesses 133 (e.g. a recess of balloon 136) shown in FIG. 5A. Positioned within each recess 133 is a port 137, configured to capture or at least engage tissue when a vacuum is applied to each port 137, such as via one or more conduits such as conduits 111 described hereabove. Recesses 133 can be sized such that port 137 is relatively flush with the surface of an expanded functional assembly 130 or is otherwise constructed and arranged to limit the radial extension of each port 137 from the outer surface of an expanded functional assembly 130, such as to allow the surface of functional assembly 130 proximate each port 137 to sufficiently contact intestinal wall tissue (e.g. to avoid “tenting” of the tissue around each port 137), and/or to avoid trauma to intestinal wall tissue proximate each port 137.

In some embodiments, catheter 100 comprises one or more ports, port 112, configured to deliver and/or extract fluids, such as to perform an insufflation or desufflation step, such as to change the level of contact between functional assembly 130 and the intestinal wall (e.g. desufflation to achieve sufficient apposition between functional assembly 130 and the intestinal wall to ablate target tissue), as described herein. Catheter 100 of FIG. 5B comprises port 112a positioned on shaft 110 proximal to functional assembly 130 and port 112b positioned distal to functional assembly 130. Ports 112a and 112b are fluidly connected to conduits 111a and 111b, respectively, such that fluid can be extracted (e.g. liquids or gases extracted by console 200 described hereabove) from within the intestine by ports 112a and/or 112b, such as to desufflate the intestine proximal and/or distal to functional assembly 130. Alternatively or additionally, fluid can be delivered to the intestine by ports 112a and/or 112b, such as to insufflate the associated segment of the intestine. Catheter 100 can comprise one or more ports positioned along functional assembly 130, such as ports 137 which include openings 138 shown in FIG. 5B. Fluid can be delivered or extracted, such as to insufflate or desufflate, respectively, as described hereabove in reference to ports 112a and 112b. Alternatively or additionally, ports 137 including openings 138 can be configured to capture or at least frictionally engage tissue (e.g. wall tissue of the intestine), such as to complete a tissue expansion procedure and/or to anchor the distal portion of catheter 100, each as described herein. In some embodiments, functional assembly 130 of FIGS. 5A-B is configured to both expand one or more tissue portions and ablate one or more tissue portions. In some embodiments, ports 112a, 112b or another component of catheter 100 or system 10 (e.g. a working channel of introduction device 50) is configured to automatically insufflate and/or desufflate, such as an insufflation and/or desufflation triggered by a recording by a sensor of system 10 (e.g. a sensor as described herein, and whose signal is processed by algorithm 251 to automatically initiate the delivery and/or extraction of fluids from the intestine).

In some embodiments, catheter 100 comprises a bulbous distal tip, such as a tip configured to be inflated or otherwise expanded, such as inflatable tip 115′ shown in FIG. 5A-B, which can comprise a balloon or other expandable structure. Inflatable tip 115′ can be fluidly attached to conduit 111c which can travel proximally to be attached to an inflation source, such as a pumping assembly 225 and reservoir 220 of console 200 described hereabove in reference to FIG. 1. Inflatable tip 115′ can be configured to expand to a diameter of at least 4 mm and/or a diameter of no more than 15 mm, such as an inflation that occurs after inflatable tip 115′ exits a lumen (e.g. a lumen of an introduction device such as endoscope 50a or sheath 90 described hereabove in reference to FIG. 1).

In some embodiments, catheter 100 comprises functional element 119 positioned in, on and/or within shaft 110. Functional element 119 can comprise a heating or cooling element configured to modify and/or control the temperature of fluid entering balloon 136.

In some embodiments, catheter 100 of FIGS. 5A-B comprises one or more sensors, such as one or more functional elements 109, 119 and/or 139 described hereabove in reference to FIG. 1, that have been configured as a sensor. These one or more sensors can be configured to provide a signal, such as a signal used to adjust one or more console 200 settings (e.g. console settings 201) of the present inventive concepts. In some embodiments, functional assembly 130 comprises one or more functional elements, such as functional element 139a, 139b and/or 139c described hereabove in reference to FIG. 1, such as a functional element constructed and arranged to perform a therapeutic and/or diagnostic medical procedure, as described herein.

Referring now to FIG. 6, a flow chart of a method of treating a patient is illustrated, consistent with the present inventive concepts. Method 2000 of FIG. 6 can be performed using system 10 of the present inventive concepts, such as by using catheters 100, 20, 30, and/or 40 as described hereabove. Method 2000 will be described using system 10 and catheter 100 as described hereabove in reference to FIGS. 1, 2, 3, 4, and/or 5A-B.

Throughout method 2000, system 10 (e.g. via user interface 205 of console 200) can provide (e.g. to an operator) information related to the fluids in the various reservoirs 220, such as volume, pressure, and/or temperature information. Based on the provided information, the procedure may be aborted, modified, or proceeded as intended (e.g. manually by the operator and/or automatically by system 10). The provided information can relate to ablative fluid (e.g. hot or cold ablative fluid), neutralizing fluid (e.g. cold or warm, respectively, neutralizing fluid), injectate 221, and/or other fluid.

In STEP 2010, a patient is selected for treatment. The patient can be selected to treat one or more of the medical conditions described herein. In some embodiments, the selected patient is inflicted with Type 2 diabetes and another medical condition, such as NAFLD/NASH. One or more patient diagnostic tests can be performed such as to include or exclude a potential patient.

In STEP 2020, a visualization device is inserted into the patient by an operator of system 10 (e.g. a clinician of the patient). For example, the visualization device can comprise an endoscope (e.g. endoscope 50a described hereabove). Alternatively or additionally, the visualization device inserted in STEP 2020 can be a treatment device of the present inventive concepts, such as catheter 100 described hereabove, such as a treatment device that includes a camera or other visualization assembly. In these embodiments, the inserted catheter 100 has already been prepared for insertion via performance of STEP 2050 described herebelow.

In some embodiments, guidewire 60 is inserted into the patient (e.g. via a working channel of an endoscope and/or a guidewire lumen of a treatment device). Guidewire 60 can be used to introduce catheter 100 (in STEP 2020 or otherwise).

In some embodiments, the visualization device comprises an endoscope with a scope cap, such as cap 53 described hereabove in reference to FIG. 1. Scope cap 53 can prevent tissue (e.g. duodenal or other luminal wall tissue) from collapsing in front of a camera of the endoscope, such tissue collapse undesirably limiting the view provided by the visualization device.

The visualization device and other devices inserted in the various steps below, can be inserted into the patient via the mouth, such as to enter the small intestine by passing through the stomach. Alternatively, the device can be inserted via a surgical incision through the skin, and/or via minimally invasive access tools (e.g. one or more laparoscopic ports).

In STEP 2030, an optional step of marking non-target tissue is performed. Using the visualization device inserted in STEP 2020, the operator can identify the ampulla of Vater, such as to mark the ampulla of Vater to allow rapid, simplified visualization of the ampulla of Vater in later steps (e.g. to avoid adversely affecting the ampulla of Vater and its neighboring tissue). In some embodiments, the ampulla of Vater is visualized using a side-viewing visualization device (e.g. an endoscope with side-viewing capability). In some embodiments, the ampulla of Vater is marked through implantation of a marker, such as marker 430 described hereabove, such as a temporarily implantable marker, such as a hemostasis clip. Marker 430 can comprise a radiopaque marker (e.g. to be visualized by a fluoroscope), an ultrasonically visible marker (e.g. to be visualized by an ultrasound imaging device), and/or a magnetic marker. Marker 430 can comprise biocompatible ink.

In some embodiments, one or more patient screening procedures are performed in STEP 2030, such as to confirm that the target tissue to be treated, and/or tissue proximate the target tissue, is free of disease or other undesired conditions. If an undesired condition is identified, the procedure can be aborted (e.g. via step 2140 described herebelow).

In STEP 2040, the visualization device inserted in STEP 2020 can be removed from the patient, such as when the visualization device comprises endoscope 50a or other body introduction device 50. Removal of this type of visualization device can be performed leaving a guidewire (e.g. guidewire 60) in place. Alternatively, the visualization device inserted in STEP 2020 comprises a treatment device of the present inventive concepts, and the treatment device, such as a catheter 100 remains in the patient.

In STEP 2050, a treatment device, such as catheter 100, is prepared for insertion into the patient. In some embodiments, catheter 100 comprises the visualization device of STEP 2020, and STEP 2050 is performed prior to STEP 2020.

Catheter 100 is attached to console 200, such as via connecting assembly 300, and one or more procedures are performed such as to remove air from one or more lumens, balloons, and/or other spaces within catheter 100. In some embodiments, catheter 100 is prepared using method 3000 described herebelow in reference to FIG. 7.

In some embodiments, functional assembly 130 comprises balloon 136, and after the final procedure of STEP 2050 is performed, balloon 136 is filled with a small, but non-zero volume of fluid, at a pressure less than full vacuum, such that functional assembly 130 is in a preferred “translation state”, as described herebelow in reference to FIG. 7. This translation state provides numerous advantages for safe and effective translation of catheter 100 in the duodenum and other segments of the GI tract of the patient, also as described herebelow in reference to FIG. 7.

In STEP 2060, catheter 100 is inserted into the patient (e.g. if not already inserted in STEP 2020). In some embodiments, catheter 100 is inserted with the corresponding functional assembly 130 in the translation state described hereabove in STEP 2050 and herebelow in reference to method 3000 of FIG. 7. In some embodiments, catheter 100 is inserted over a guidewire (e.g. using fluoroscopic guidance), such as guidewire 60 which can be already in place as described hereabove.

In some embodiments, such as when duodenal mucosal tissue is to be treated (i.e. the target tissue comprises duodenal mucosa), functional assembly 130 of catheter 100 is positioned proximate the duodenal bulb or segment D1 of the duodenum.

In some embodiments, catheter 100 is inserted after a body introduction device, such as endoscope 50a, has been recently removed (e.g. in STEP 2040).

In some embodiments, after catheter 100 is introduced into the patient in STEP 2060, endoscope 50a is introduced (e.g. reintroduced) into the patient as well. Subsequent translations of catheter 100 can be performed with simultaneous translation of endoscope 50a.

In STEP 2070, a treatment assembly configured to perform a submucosal tissue expansion, such as functional assembly 130 of catheter 100, is positioned at a first location in the patient's small intestine, such as a location in the duodenum distal to the pylorus and proximal to the Ligament of Treitz. Alternatively or additionally, other GI locations can be selected for tissue expansion (e.g. submucosal tissue expansion). During positioning, catheter 100 (e.g. functional assembly 130) can be in a translation state as described herein.

In STEP 2080, a submucosal tissue expansion is performed, such as via functional assembly 130 of catheter 100, the expansion performed at the location established in STEP 2070.

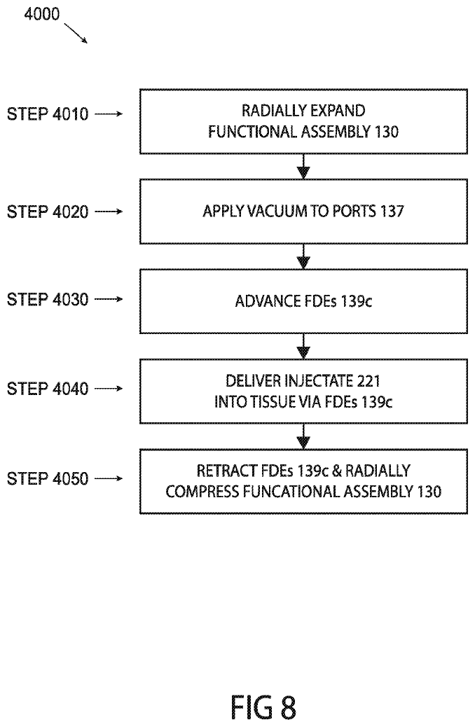

The tissue expansion performed in STEP 2080 can be performed using method 4000 described herebelow in reference to FIG. 8.

In STEP 2090, the treatment catheter is translated, such as a translation of catheter 100. In some embodiments, catheter 100 is translated such as to cause a corresponding translation of functional assembly 130 that is approximately one-half of the length of functional assembly 130 (e.g. approximately lcm when functional assembly 130 comprises a length of approximately 2 cm). In some embodiments, catheter 100 is translated by the length of the previously expanded submucosal tissue (e.g. approximately 1 cm), such that an additional length of submucosal tissue can be expanded. In some embodiments, functional assembly 130 is translated distally (e.g. more distal in the duodenum, further away from the ampulla of Vater toward but not passing the ligament of Treitz). Alternatively, functional assembly 130 is translated proximally. During translation, catheter 100 (e.g. functional assembly 130) can be in a translation state as described herein.

In STEP 2100, an optional step of another submucosal tissue expansion is performed, such as via functional assembly 130 of catheter 100. The tissue expansion is performed at the location established in STEP 2090. Catheter 100 (e.g. functional assembly 130) can be in a translation state as described herein.

The tissue expansion performed in STEP 2100 can be performed using method 4000 described herebelow in reference to FIG. 8.

The tissue expansion performed in STEP 2100 can be performed at a duodenal or other GI location that is proximate, yet distal to the location of tissue expansion performed in step 2080. Alternatively, the tissue expansion performed in STEP 2100 can be proximal to the location of STEP 2080.

In STEP 2110, a tissue treatment procedure is performed, such as via functional assembly 130 of catheter 100. The tissue treatment procedure can be performed in the same location of the tissue expansion performed in STEP 2100 (e.g. without translation of functional assembly 130).

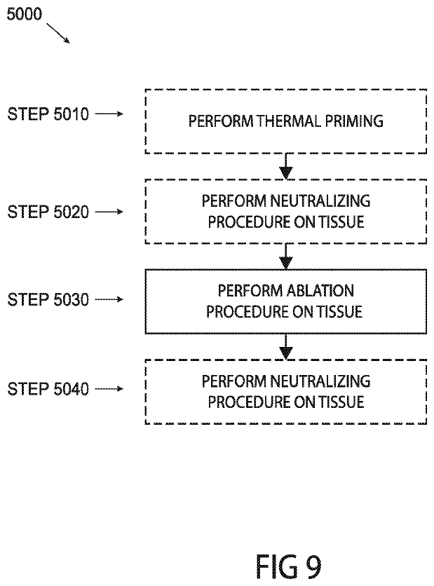

The tissue treatment performed in STEP 2110 can be performed using method 5000 described herebelow in reference to FIG. 9. In some embodiments, prior to performing method 5000, catheter 100 and functional assembly 130 are established in the translation state described herein.

The tissue treatment performed in STEP 2110 can include a neutralizing procedure and an ablation procedure, such as is described hereabove in reference to FIGS. 1 and/or 2. In some embodiments, a neutralizing procedure (e.g. a cooling or warming procedure) is performed prior to and/or after an ablation procedure (e.g. a heat or cryogenic ablation procedure, respectively) at a single axial location of the GI tract (e.g. and repeated for multiple axial locations). In some embodiments, a neutralizing procedure (e.g. a cooling or warming procedure) is performed only after (i.e. not prior to) an ablation procedure (e.g. a heat or cryogenic ablation procedure, respectively) at a single axial location of the GI tract (e.g. and repeated for multiple axial locations). In other embodiments, a neutralizing procedure (e.g. a cooling or warming procedure) is performed both prior to and after an ablation procedure (e.g. a heat or cryogenic ablation procedure, respectively) at a single axial location of the GI tract (e.g. and repeated for multiple axial locations).

In STEP 2120, a decision is made related to performing additional tissue treatments. If additional tissue treatments are desired, STEP 2130 is performed. If the procedure is complete, STEP 2140 is performed. In some embodiments, at least two, three, four, five, or six tissue treatments are performed. In some embodiments, at least 60 mm of cumulative axial length of duodenum is treated, such as to achieve a desired therapeutic benefit as described herein. The at least 60 mm of cumulative axial length can be treated via a single treatment step (e.g. a single ablation using functional assembly 130), or via multiple treatment steps (e.g. at least 3 ablations, at least 4 ablations, and/or at least 5 ablations using functional assembly 130). In these embodiments, functional assembly 130 can comprise a treatment length of at least 10 mm, such as a treatment length of no more than 100 mm.

In STEP 2130, catheter 100, including functional assembly 130, is translated to a new location within the GI tract, such as a location approximately lcm distal to the current location. Alternatively, functional assembly 130 can be translated proximally (e.g. 1 cm proximally). Subsequently, STEP 2080 is repeated.

In STEP 2140, the treatment device, and any other device (e.g. endoscope 50a and/or guidewire 60) is removed from the patient, and the procedure is complete.

In some embodiments, the tissue expansion procedures (STEPS 2080 and 2100) and the tissue treatment procedures (STEP 2110) are performed with the same catheter, such as catheter 100 and/or 40 described hereabove. In other embodiments, the tissue expansion procedures (STEPS 2080 and 2100) are performed with a first catheter, such as catheter 20 described hereabove, and the tissue treatment procedures (STEP 2110) are performed with a second, different catheter, such as catheter 100 of FIG. 2.

Referring now to FIG. 7, a flow chart of a method of preparing a treatment device is illustrated, consistent with the present inventive concepts. Method 3000 of FIG. 7 can be performed using system 10 of the present inventive concepts, such as by using catheters 100, 20, 30, and/or 40 as described hereabove. Method 3000 is described using system 10 and catheter 100 as described hereabove in reference to FIGS. 1, 2, 3, 4, and/or 5A-B. Method 3000 is performed on a catheter that has been attached to console 200, such as via connecting assembly 300 as described hereabove in reference to FIGS. 1 and 2.

Throughout method 3000, system 10 (e.g. via user interface 205 of console 200) can provide (e.g. to an operator) information related to the fluids in the various reservoirs 220, such as volume, pressure, and/or temperature information. Based on the provided information, the procedure may be aborted, modified, or proceeded as intended (e.g. manually by the operator and/or automatically by system 10). The provided information can relate to ablative fluid (e.g. hot or cold ablative fluid), neutralizing fluid (e.g. cold or warm, respectively, neutralizing fluid), and/or other fluid.

In STEP 3010, a fluid fill procedure is performed, such as to fully or partially fill functional assembly 130 (e.g. balloon 136) with fluid. The fluid fill procedure can be performed: for a pre-determined period of time; until a particular volume of fluid is delivered into functional assembly 130; and/or until a pre-determined pressure is achieved within functional assembly 130. The delivery of fluid can be performed at a particular pressure or range of pressures, and/or at a particular flow rate or range of flow rates. In some embodiments, as fluid is delivered into functional assembly 130 (e.g. via one or more lumens or other conduits of catheter 100), fluid is simultaneously evacuated (e.g. slowly removed) from functional assembly 130 (e.g. via one or more lumens or other conduits of catheter 100), such that functional assembly 130 does significantly expand during the process. The fluid delivered to functional assembly 130 in STEP 3010 can be relatively cold fluid, such as fluid that is less than body temperature and/or less than room temperature. For example, the fluid provided by a reservoir 220 of console 200 can contain a fluid that is also a neutralizing fluid configured to perform a pre-cool and/or post-cool of an ablation treatment, such as is described herebelow in reference to method 5000 of FIG. 9.

The delivery and removal of various fluids to and/or from catheter 100 can be performed by one or more pumping assemblies 225 of console 200.

In STEP 3020, a fluid evacuation procedure is performed, such as to fully or partially evacuate functional assembly 130 (e.g. balloon 136) of fluid. The fluid evacuation procedure can be performed: for a pre-determined period of time (e.g. for less than 15 seconds, for less than 10 seconds, and/or for approximately 6 seconds); until a particular volume of fluid is removed from and/or remains within functional assembly 130; and/or until a pre-determined pressure is achieved within functional assembly 130. The evacuation can be performed at a particular pressure or range of pressures, and/or at a particular flow rate or range of flow rates. In some embodiments, as fluid is evacuated from functional assembly 130 (e.g. via one or more lumens or other conduits of catheter 100) until a particular volume remains within functional assembly 130 (e.g. within balloon 136). Removal of fluids can be performed by one or more pumping assemblies 225 of console 200. In some embodiments, the pressure within functional assembly 130 is near full vacuum at the end of STEP 3020.

In some embodiments, STEPS 3010 and 3020 are repeated one or more times, prior to performing STEP 3030, such as when catheter 100 is initially prepared for insertion into the patient and STEPS 3010 and 3020 are performed at least two times each.

In STEP 3030, a pressure setting procedure is performed, which establishes functional assembly 130 in a preferred “translation state” (e.g. a state in which translation of functional assembly 130 within the GI tract is safe, effective, and relatively easy). In STEP 3030, the pressure within functional assembly 130 (e.g. within balloon 136) is brought to a particular level. Alternatively or additionally, a particular volume (e.g. a minimal volume) of fluid is caused to remain within functional assembly 130.

In some embodiments, prior to performing STEP 3030, the pressure within functional assembly 130 is at or near full vacuum (e.g. as caused in STEP 3020). In STEP 3030, fluid can be delivered (and/or evacuated) such as to cause the pressure within functional assembly 130 to reach a target level related to the desired translation state. In some embodiments, the target level is below room pressure, such as at least 1 psi below room pressure (−1 psi), at least 2 psi below room pressure, or approximately −2.7 psi. Establishing a slightly negative pressure causes functional assembly 130 to be partially compacted, but not to the extent that significant rigidity occurs. In some embodiments, the translation state target level for the pressure within functional assembly 130 is no more than 5 psi below room pressure, or no more than 4 psi below room pressure. In other embodiments, the target pressure level for functional assembly 130 is less than 1 psi (i.e. 1 psi above room pressure), or less than 0.5 psi, and/or the translation state is established via a maximum volume contained within functional assembly 130, such as a volume less than 5%, or less than 10% of the “full volume” of balloon 136 (e.g. the volume to rigidly inflate a relatively non-compliant balloon 136, or the volume to inflate a compliant balloon without significantly stretching the balloon), the maximum pressure and/or volume establishing a limited (e.g. small) expansion of functional assembly 130. In some embodiments, the volume of fluid in balloon 136 during the transition state is less than 3 ml, 2 ml, or 1 ml.

Advantages of the translation state established for catheter 100 in STEP 3030 are that functional assembly 130 (e.g. including balloon 136 and ports 137) is established with a relatively low profile (e.g. a relatively minimal diameter surrounds shaft 110), and its components in a relatively flexible condition (e.g. not fully compacted via a complete vacuum, such that the components of functional assembly 130 are able to move with relatively low force applied). In these low profile, non-rigid states, ease of translation of functional assembly 130 is maximized or at least improved.

In some embodiments, establishing of the translation state of a treatment device (e.g. catheter 100) via method 3000 is performed between each tissue treatment (e.g. ablation) step and a subsequent submucosal tissue expansion step. For example, method 3000 can be performed after completion of STEP 2110 and prior to a (repeated) STEP 2080, each of method 2000 of FIG. 6 described hereabove.

Referring now to FIG. 8, a flow chart of a method of expanding tissue with a treatment device is illustrated, consistent with the present inventive concepts. Method 4000 of FIG. 8 can be performed using system 10 of the present inventive concepts, such as by using catheters 100, 20, 30, and/or 40 as described hereabove. Method 4000 is described using system 10 and catheter 100 as described hereabove in reference to FIGS. 1, 2, 3, 4, and/or 5A-B. Method 4000 is described using a catheter 100 that has been attached to console 200, such as via connecting assembly 300 as described hereabove in reference to FIGS. 1 and 2. Delivery and removal of fluids of method 4000 can be performed by one or more pumping assemblies 225 of console 200.

Throughout method 4000, system 10 (e.g. via user interface 205 of console 200) can provide (e.g. to an operator) information related to injectate 221 in one or more reservoirs 220, such as volume, pressure, and/or temperature information. Based on the provided information, the procedure may be aborted, modified, or proceeded as intended (e.g. manually by the operator and/or automatically by system 10).

In STEP 4010, functional assembly 130 of catheter 100 is radially expanded, such as by the delivery of fluid into balloon 136. For example, fluid can be delivered from one or more reservoirs 220 by a pumping assembly 225. The fluid delivered in STEP 4010 can be performed: for a pre-determined period of time; until a particular volume of fluid is delivered into functional assembly 130; and/or until a pre-determined pressure is achieved within functional assembly 130. The delivery of fluid can be performed at a particular pressure or range of pressures, and/or at a particular flow rate or range of flow rates. In some embodiments, as fluid is delivered into functional assembly 130 (e.g. via one or more lumens or other conduits of catheter 100), fluid is simultaneously evacuated (e.g. slowly removed) from functional assembly 130 (e.g. via one or more lumens or other conduits of catheter 100), such that functional assembly 130 does significantly expand during the process. The fluid delivered to functional assembly 130 in STEP 4010 can be relatively cold fluid, such as fluid that is less than body temperature and/or less than room temperature (e.g. fluid provided by a reservoir 220 of console 200) that contains a neutralizing fluid configured to perform a pre-cool and/or post-cool of an ablation treatment that includes and/or generates heat (e.g. a hot fluid ablation, an RF ablation, a light ablation, and/or an ultrasound ablation). Delivery and removal of fluids can be performed by one or more pumping assemblies 225 of console 200.

In some embodiments, a fixed volume of fluid is delivered to functional assembly 130, such as a volume of at least 4 ml, at least 6 ml, or approximately 8 ml. In some embodiments, fluid is delivered until functional assembly 130 is in relatively close apposition to the wall of the GI tract within which functional assembly 130 is positioned (e.g. automatically by system 10 or manually by an operator).

In STEP 4020, vacuum is applied to one or more (e.g. all) ports 137 of functional assembly 130, such that tissue proximate each port 137 is drawn into a cavity of port 137. In some embodiments, the pressure applied to port 137 is monitored (e.g. via a location within console 200, connecting assembly 300, and/or catheter 100). The monitoring of the pressure can be used to confirm that the pressure maintains a minimum vacuum (e.g. at least 2 psi, at least 4 psi, or at least 6 psi below room pressure). Alternatively or additionally, the pressure can be monitored to confirm that the vacuum level is relatively stable, such as a stability correlating to a pressure that does not vary more than 0.3 psi, 0.2 psi, and/or 0.1 psi within a time window of at least 2 seconds, at least 3 seconds, and/or at least 5 seconds. If the minimum vacuum level, or stability level is not maintained, system 10 can be configured to enter an alert state (e.g. a state in which the operator is notified and/or further treatment steps are prevented until resolution is achieved).

In STEP 4030, one or more fluid delivery elements 139c are advanced (e.g. multiple fluid delivery elements 139c that are simultaneously or sequentially advanced) into the tissue captured within each corresponding port 137. In some embodiments, multiple fluid delivery elements 139c are advanced by a single control (e.g. a control 104 on handle 102 of catheter 100, as described herein). In some embodiments, two or more fluid delivery elements 139 are advanced by separate, individual controls (e.g. two or more controls 104).

In STEP 4040, injectate 221 is delivered into the submucosal tissue by one or more needles or other fluid delivery elements 139c (e.g. into the tissue captured within each port 137). Injectate 221 is provided via one or more reservoirs 220 and delivered by one more pumping assembly 225, such as is described hereabove in reference to FIGS. 1 and/or 2. In some embodiments, a fixed volume of fluid is introduced through each fluid delivery element 139c, such as at least 3 ml, at least 5 ml, at least 7 ml, or approximately 10 ml injected into tissue via at least two, at least three, or at least four fluid delivery elements 139c.

In some embodiments, pressure within the fluid pathway containing injectate 221 (e.g. within each associated reservoir 220 such as a syringe or other reservoir) is monitored during the delivery of injectate 221 to tissue. In some embodiments, injectate 221 is delivered at a flow rate than prevents the pressure within the fluid pathway from exceeding a maximum level, such as a level of no more than 150 psi, or no more than 100 psi at a fluid pathway location proximate console 200. In some embodiments, multiple fluid delivery elements 139c (e.g. needles) are each fluidly attached to individual, separate reservoirs 220, via separate fluid pathways, and if the associated fluid pathway pressure for a single fluid delivery element 139c exceeds the maximum level, the flow rate of injectate 221 delivery is reduced (e.g. reduced for all fluid delivery elements 139c). Pressure measurements above the maximum could relate to an occlusion or other restriction in the fluid pathway between console 200 and fluid delivery elements 139c and exceeding the pressure can result in system 10 entering an alert state. Configuration of system 10 to prevent exceeding the maximum pressure provides a safety measure (avoiding excessive pressure of injectate 221 delivery into the patient). In some embodiments, the pressure within each flow pathway containing injectate 221 is confirmed to be above a minimum pressure (e.g. such as a pressure of at least 20 psi). Pressure below the minimum can indicate air in the fluid pathway, or a leak, and system 10 can be configured to enter an alert state if the minimum threshold is exceeded.

In some embodiments, system 10 (via console 200) is configured to maintain a constant volume within functional assembly 130 (e.g. within balloon 136) throughout the injection of injectate 221 into tissue. For example, the volume within balloon 136 can be at a level less that the volume of balloon 136 when it is fully expanded. In some embodiments, the volume is no more than 90% of the full volume of balloon 136, such as no more than 80% of the full volume, or no more than 70% of the full volume (e.g. balloon 136 is filled with 8 ml when the full volume is 12 ml). In some embodiments, system 10 is configured to enter an alert state if the volume within functional assembly 130 is below a minimum and/or above a maximum.

In some embodiments, system 10 (via console 200) is configured to regulate the pressure (e.g. ensure the pressure is above a minimum and/or below a maximum) within functional assembly 130 (e.g. within balloon 136) during injection of injectate 221 into tissue. In some embodiments, system 10 is configured to enter an alert state if the pressure within functional assembly 130 is below a minimum and/or above a maximum.

In STEP 4050, all fluid delivery elements 139c are retracted, and functional assembly 130 is radially compressed. Retraction of fluid delivery elements 139c can be performed in a similar, typically opposite direction, to the method used to deploy them in STEP 4030 (e.g. via one or more controls 104 of handle 102 of catheter 100). Functional assembly 130 can be radially compressed via evacuation of the fluid within functional assembly 130, via one or more pumping assemblies 225 as described herein. In some embodiments, functional assembly 130 is radially compressed by evacuating a fixed volume of fluid (e.g. from balloon 136), such as the same or at least a similar volume to that introduced into functional assembly 130 in STEP 4010 (e.g. a volume of at least 4 ml, at least 6 ml, or approximately 8 ml).

Referring now to FIG. 9, a flow chart of a method of ablating or otherwise treating tissue with a treatment device is illustrated, consistent with the present inventive concepts. Method 5000 of FIG. 9 can be performed using system 10 of the present inventive concepts, such as by using catheters 100, 20, 30, and/or 40 as described hereabove. Method 5000 is described using system 10 and catheter 100 as described hereabove in reference to FIGS. 1, 2, 3, 4, and/or 5A-B. Method 5000 is described using a catheter 100 that has been attached to console 200, such as via connecting assembly 300 as described hereabove in reference to FIGS. 1 and 2. Delivery and removal of fluids of method 5000 can be performed by one or more pumping assemblies 225 of console 200.

Throughout method 5000, system 10 (e.g. via user interface 205 of console 200) can provide (e.g. to an operator) information related to the fluids in the various reservoirs 220, such as volume, pressure, and/or temperature information. Based on the provided information, the procedure may be aborted, modified, or proceeded as intended (e.g. manually by the operator and/or automatically by system 10). The provided information can relate to ablative fluid (e.g. hot or cold ablative fluid), and/or neutralizing fluid (e.g. cold or warm, respectively, neutralizing fluid).

In the various steps of method 5000, a reservoir 220 can be filled with an ablative fluid at an elevated temperature, such as a temperature of at least 90° C., at least 93° C., or approximately 96° C. Alternatively or additionally this elevated temperature ablative fluid can be maintained at a temperature of no more than 99° C., such as no more than 98° C., or no more than 97° C. Another reservoir 220 can be filled with a neutralizing fluid that is maintained at a temperature less than body temperature, such as a temperature of approximately room temperature. Alternatively or additionally, a reservoir 220 can be filled with a chilled fluid. The chilled fluid can be maintained at a temperature of no more than 30° C., or no more than 25° C. Alternatively or additionally, this chilled fluid can be maintained at a temperature below room temperature but above 5° C., such as above 7.5° C., or above 9° C.

In STEP 5010, an optional step of a thermal priming procedure is performed on one or more of the fluid pathways of console 200, connecting assembly 300 (if present), and catheter 100. In some embodiments, the fluid pathways of connecting assembly 300 are warmed, such as to a temperature of at least 60° C., 70° C., or 80° C., such as a temperature of approximately 86° C. In these embodiments, fluid pathways of catheter 100 can also be warmed, or not.

In STEP 5020, an optional step of performing a pre-ablation neutralizing procedure on tissue is performed (e.g. to tissue in close proximity to functional assembly 130 and/or tissue proximate and/or somewhat remote from this tissue). For example, a cooling fluid can be delivered to functional assembly 130, such as when the ablation of STEP 5030 includes and/or generates heat, such as when the ablation includes a hot fluid ablation, an electromagnetic energy ablation (e.g. an RF ablation), a light energy ablation (e.g. a laser ablation), and/or a sound energy ablation (e.g. a high intensity or other ultrasound ablation).

Upon activation by an operator via user interface 205, neutralizing fluid is introduced into functional assembly 130 (e.g. into balloon 136).

The neutralizing fluid can be delivered to functional assembly 130: for a pre-determined period of time; until a particular volume of fluid is delivered into functional assembly 130; and/or until a pre-determined pressure is achieved within functional assembly 130. The delivery of fluid can be performed at a particular pressure or range of pressures, and/or at a particular flow rate or range of flow rates. In some embodiments, as neutralizing fluid is delivered into functional assembly 130 (e.g. via one or more lumens or other conduits of catheter 100), fluid is simultaneously evacuated from functional assembly 130 (e.g. via one or more lumens or other conduits of catheter 100), at flow rates such that functional assembly 130 remains expanded (e.g. remains in contact with surrounding mucosal tissue of the duodenum or other GI mucosal tissue), but fluid within functional assembly 130 is recirculated.

Once functional assembly 130 is filled with the neutralizing fluid (e.g. for a time period of no more than 5 seconds, no more than 4 seconds, and/or no more than 3 seconds), that particular volume of neutralizing fluid can remain in place (e.g. without removal or replacement) throughout the remaining portion of STEP 5020, and/or it can be recirculated, as described herein, for the remaining portion of STEP 5020.

Tissue proximate functional assembly 130 is cooled or otherwise neutralized as long as neutralizing fluid is maintained within functional assembly 130 (e.g. in a stagnant or recirculating manner), and functional assembly 130 is in relative contact with the tissue.

In some embodiments, neutralizing fluid is delivered to functional assembly 130 in a recirculating manner, for a pre-determined time period, such as a time period of at least 5 seconds, 10 seconds, or 15 seconds. In these embodiments, for an initial period (e.g. a period of approximately 2 seconds), fluid is not evacuated from functional assembly 130, allowing functional assembly 130 to radially expand to contact tissue. Subsequently (e.g. for at least the next 3 seconds, 8 seconds, or 12 seconds), functional assembly 130 is in contact with mucosal tissue and neutralizing fluid cools the contacted mucosal tissue as well as other tissue in relative proximity to the contacted mucosal tissue (e.g. neighboring mucosal tissue, as well as deeper tissues including the neighboring submucosal tissue, gastrointestinal adventitia, the tunica serosa, and tunica muscularis).

During this tissue neutralizing procedure, one or more fluid pathway temperatures can be monitored, as described herein, such as to change temperature in a closed-loop fashion, and/or to enter an alert state if a temperature threshold is exceeded.

During this tissue neutralizing procedure, the pressure within one or more fluid pathways can be monitored, such as to adjust the pressure in a closed-loop fashion, and/or to enter an alert state if a pressure threshold is exceeded. For example, pressure below a minimum can represent a break of balloon 136 and/or other leak in the fluid pathway. Pressure above a maximum can represent an occlusion or restriction (e.g. a kink in catheter 100) has occurred.

Temperature and/or pressure can be monitored by one or more temperature sensor and/or pressure sensor-based functional elements of console 200, connecting assembly 300, and/or catheter 100, as described in detail hereabove in reference to FIGS. 1 and/or 2.

While STEP 5020 has primarily been described using a cooling fluid, in alternative embodiments, a warming fluid can be delivered to functional assembly 130 (e.g. to neutralize a cryogenic ablation) or an agent configured to neutralize a chemical ablation can be delivered directly to the mucosal tissue surface (e.g. a non-target tissue surface).

In STEP 5030, an ablation or other tissue treatment procedure is performed on target tissue (e.g. to tissue in close proximity to functional assembly 130 and/or tissue proximate this tissue). For example, an elevated temperature ablative fluid can be delivered to functional assembly 130, such as when the neutralizing fluid of STEP 5020 comprised fluid at a temperature below body temperature.

Ablative fluid is introduced into functional assembly 130 (e.g. into balloon 136), via manual activation by an operator or automatically by system 10 (e.g. an automatic initiation when STEP 5020 is completed).

The ablative fluid can be delivered to functional assembly 130: for a pre-determined period of time; until a particular volume of fluid is delivered into functional assembly 130; and/or until a pre-determined pressure is achieved within functional assembly 130. The delivery of fluid can be performed at a particular pressure or range of pressures, and/or at a particular flow rate or range of flow rates. In some embodiments, as neutralizing fluid is delivered into functional assembly 130 (e.g. via one or more lumens or other conduits of catheter 100), fluid is simultaneously evacuated from functional assembly 130 (e.g. via one or more lumens or other conduits of catheter 100), at flow rates such that functional assembly 130 remains expanded (e.g. remains in contact with surrounding mucosal tissue of the duodenum or other GI mucosal tissue), but fluid within functional assembly 130 is recirculated.

Once functional assembly 130 is filled with the ablative fluid (e.g. for a time period of no more than 5 seconds, no more than 4 seconds, and/or no more than 3 seconds), that particular volume of ablative fluid can remain in place (e.g. without removal or replacement) throughout the remaining portion of STEP 5030, and/or it can be recirculated, as described herein, for the remaining portion of STEP 5030.

Tissue proximate functional assembly 130 is ablated or otherwise treated as long as ablative fluid is maintained within functional assembly 130 (e.g. in a stagnant or recirculating manner), and functional assembly 130 is in relative contact with the tissue.

In some embodiments, ablative fluid is delivered to functional assembly 130 in a recirculating manner, for a pre-determined time period, such as a time period of at least 5 seconds, 7 seconds, or 10 seconds.

In some embodiments, ablative fluid is introduced into functional assembly 130 immediately after completion of STEP 5020, without evacuation of the neutralizing fluid introduced in STEP 5020.

In some embodiments, ablative fluid is introduced into a tissue-contacting functional assembly 130 in a recirculating manner, for a calculated time period, the “ablation time”, that is based on the temperature of cold neutralizing fluid delivered to functional assembly 130 in STEP 5020. For example, the colder the temperature of the neutralizing fluid, the longer the ablation time, and vice versa. Alternatively or additionally, the ablation time can be based on the time that the neutralizing fluid cools (e.g. extracts heat from) the tissue, the “neutralizing time”. In some embodiments, the ablation time is also based on the temperature of the ablative fluid (e.g. the hotter the fluid the shorter the ablation time, and vice versa). In some embodiments, the ablation time is based on the information provided in Table F below, such as when the neutralizing time is at least 5 seconds, at least 10 seconds, or approximately 15 seconds:

TABLE F

| Cold Reservoir Temp (° C.) | Ablation Time (seconds) |
|---|---|
| 9.0-10.9 | 10.0 |
| 11.0-12.9 | 9.8 |
| 13.0-14.9 | 9.6 |
| 15.0-16.9 | 9.4 |
| 17.0-18.9 | 9.2 |
| 19.0-20.9 | 9.0 |
| 21.0-22.9 | 8.8 |
| 23.0-25.0 | 8.6 |

During this tissue ablation procedure, one or more fluid pathway temperatures can be monitored, as described herein, such as to change temperature in a closed-loop fashion, and/or to enter an alert state if a temperature threshold is exceeded.

During this tissue ablation procedure, the pressure within one or more fluid pathways can be monitored, such as to adjust the pressure in a closed-loop fashion, and/or to enter an alert state if a pressure threshold is exceeded. For example, pressure below a first minimum can represent a break of balloon 136 and/or other leak in the fluid pathway. Pressure below a second minimum (similar or dissimilar to the first), can represent that functional assembly 130 is not in adequate contact with the mucosal tissue. Pressure above a maximum can represent an occlusion or restriction (e.g. a kink in catheter 100) has occurred.

Temperature and/or pressure can be monitored by one or more temperature sensor and/or pressure sensor-based functional elements of console 200, connecting assembly 300, and/or catheter 100, as described in detail hereabove in reference to FIGS. 1 and/or 2.

While STEP 5020 has primarily been described using a cooling fluid, in alternative embodiments, a warming fluid can be delivered to functional assembly 130 (e.g. to neutralize a cryogenic ablation) or an agent configured to neutralize a chemical ablation can be delivered directly to the mucosal tissue surface (e.g. a non-target tissue surface).

As described in reference to a heat ablation, one or more ablation parameters of STEP 5030 can be based on one or more neutralizing parameters of STEP 5020, and vice versa. For example, a cryogenic ablation time can be based on a warming neutralizing temperature and/or neutralizing time. A chemical ablation concentration (e.g. pH), can be based on the concentration of a neutralizing procedure (e.g. a neutralizing procedure performed prior to and/or after the ablation step). An electromagnetic, light, and/or ultrasound ablation can be configured (e.g. adjustment of energy delivery and/or ablation time), based on a neutralizing procedure parameter.

In STEP 5040, an optional step of performing a post-ablation neutralizing procedure is performed (e.g. to tissue in close proximity to functional assembly 130 and/or tissue proximate and/or somewhat remote from this tissue).

The neutralizing procedure of STEP 5040 can be similar to the neutralizing step of STEP 5020. Similarly, the neutralizing step can be performed for a fixed period of time, such as a time of at least 5 seconds, at least 10 seconds, or at least 15 seconds. The neutralizing procedure of STEP 5040 can comprise one or more parameters that are determined by the parameters of the neutralizing procedure of step 5020 and/or the ablation procedure of STEP 5030. Alternatively or additionally, the neutralizing procedure of STEP 5040 can comprise one or more parameters that are used to determine one or more parameters of the procedures of STEPS 5020 and/or 5030.

After the completion of STEP 5040, or STEP 5030 (if neutralizing procedure of STEP 5040 is not performed), fluid can be withdrawn from functional assembly 130, such as for a fixed time period (e.g. no more than 10 seconds, no more than 8 seconds, and/or approximately 6 seconds), and/or until a particular volume of fluid is evacuated. After the fluid evacuation, functional assembly 130 can be transitioned to the translation state, as described herein.

In some embodiments, the functional assembly 130 of method 2000 of FIG. 6 is configured to deliver one or more different forms of energy to target tissue, such as when functional assembly 130 comprises one or more energy delivery elements configured to deliver an energy form selected from the group consisting of: electromagnetic energy; rf energy; light energy; laser light energy; sound energy; ultrasound energy; chemical energy; and combinations thereof. The functional assembly 130 can comprise a balloon (e.g. balloon 136) and/or is can include an array of energy delivery elements (e.g. an array of balloons and/or an array of electrodes). The functional assembly 130 of method 200 can comprise a treatment length of at least 10 mm long and/or no more than 100 mm long. The functional assembly 130 can comprise an expanded diameter of at least 20 mm and/or no more than 40 mm, or no more than 30 mm.

In some embodiments, the patient identified in STEP 2010 of method 2000 of FIG. 6 has been diagnosed with a medical condition selected from the group consisting of Type 2 diabetes; Type 1 diabetes; “Double diabetes”; gestational diabetes; hyperglycemia; pre-diabetes; impaired glucose tolerance; insulin resistance; non-alcoholic fatty liver disease (NAFLD); non-alcoholic steatohepatitis (NASH); obesity; obesity-related disorder; polycystic ovarian syndrome (PCOS); hypertriglyceridemia; hypercholesterolemia; psoriasis; GERD; coronary artery disease (e.g. as a secondary prevention); stroke; TIA; cognitive decline; dementia; Alzheimer's disease; neuropathy; diabetic nephropathy; retinopathy; heart disease; diabetic heart disease; heart failure; diabetic heart failure; hirsutism; hyperandrogenism; fertility issues; menstrual dysfunction; cancer such as liver cancer, ovarian cancer, breast cancer, endometrial cancer, cholangiocarcinoma, adenocarcinoma, glandular tissue tumor(s), stomach cancer, large bowel cancer, and/or prostate cancer; diastolic dysfunction; hypertension; myocardial infarction; microvascular disease related to diabetes; sleep apnea; arthritis; rheumatoid arthritis; hypogonadism; insufficient total testosterone levels; insufficient free testosterone levels; and combinations of two or more of these. For example, system 10 and methods 2000, 3000, and/or 4000 of FIGS. 6, 7, and/or 8, respectively, can be used to treat patients with NAFLD/NASH and Type 2 diabetes, such as is described herebelow in reference to the NAFLD/NASH Study.

In some embodiments, method 2000 of FIG. 6 is performed on a patient with NAFLD/NASH and Type 2 diabetes, and one or more of the following results are achieved (e.g. at a time period of approximately three months); hepatic fat is lowered, such as by at least 10%, at least 20%, at least 30%, and/or approximately 36%; HbA1C is lowered, such as by at least 0.5%, at least 0.7%, and/or approximately 1%; both hepatic fat and HbA1C is lowered; triglycerides are lowered, such as by at least 10%, at least 20%, and/or approximately 28%; patient weight is lowered, such as by at least 2%, at least 3%, and/or at least 5% of weight present prior to performance of the procedure of the present inventive concepts (e.g. without lifestyle intervention); weight loss of approximately 3.1 kg is achieved (e.g. without lifestyle intervention); and combinations of these. In some embodiments, these and other clinical benefits described herein are achieved within three months of performance of the procedure, and the benefits are present at least six months after the performance of the procedure. In some embodiments, results achieved include both improved HbA1C (e.g. reduced at least 0.5%) and improved liver fat content (e.g. reduced at least 20%, such as determined via MRI-PDFF). In these embodiments, HbA1C can be reduced at least 0.7% and liver fat reduced at least 30%.

In some embodiments, the results achieved immediately hereabove are dependent on a minimum amount of mucosal tissue (e.g. duodenal mucosal tissue) being treated (e.g. ablated, denatured, removed, and/or otherwise treated). For example, a single or cumulative (multiple treatment) axial length of at least 3 cm, at least 6 cm, at least 8 cm, and/or at least 9 cm of duodenal mucosa is treated to achieve these clinical benefits. Alternatively, at least 30% of the post-papillary duodenal mucosa is treated.

The method 2000 of FIG. 7 and other methods of the present inventive concepts can result in (e.g. cause) one or more of the following outcomes (e.g. outcomes related to the clinical benefits described hereabove): a reduction of surface area of mucosal tissue proximate the treated locations; an altering of hormonal signaling of the intestine proximate the treated location; replacement of the treated mucosal tissue with new tissue; a reduction in iron absorption; a reduction or increase in bile acid signaling; an altering of microbiome composition; a reduction in glucose, fat, and/or amino acid signaling and/or absorption; a reduction in GIP levels in the fasting state (e.g. by at least 5%, 10%, and/or 20%); a reduction in GIP levels in the post-prandial state (e.g. by at least 5%, 10%, and/or 20%); an increase in GLP-1 levels in the post-prandial state (e.g. by at least 5%, 10%, and/or 15%); an increase in GLP-1 levels in the post-prandial state (e.g. while not significantly altering GLP-1 levels in the fasting state); and combinations of two, three, or more of these.

Applicant has conducted a particular clinical study, the “NAFLD/NASH Study”, using system 10 of the present inventive concepts. In the NAFLD/NASH study, system 10 was used to treat patients afflicted with both NAFLD/NASH and Type 2 diabetes. The NAFLD/NASH study was performed using method 2000 described hereabove in reference to FIG. 6. The treatments of the present invention were performed in 2017 and 2018, in clinical sites in the UK, Italy, Belgium, and Brazil, and include 24 patients treated. The data described immediately herebelow represent data obtained during a follow-up procedure for each patient that occurred approximately three months after the patient's mucosal tissue treatment procedure.

In the NAFLD/NASH study, system 10 was used to treat mucosal tissue of each patient's duodenum. Previous studies by applicant in patients with Type 2 diabetes have demonstrated sustained improvements in blood glucose levels and insulin resistance measures through one year of follow-up. Results of the NAFLD/NASH study demonstrate improvements in hepatic fat fraction, glycemic profiles, and lipid profiles. Data from these patients show that the metabolic benefits extend to NAFLD/NASH, lowering liver fat by 36%. Nearly 95% of treated patients showed improvement in either glucose or liver fat, with 88% of patients showing improvement in both within three months of treatment. Additional benefits from the system 10 procedure were improvement in cardiovascular risk (lowering triglyceride/HDL ratio by 28%), and in weight, with 3.1 kg weight loss unaided by any lifestyle intervention. As with applicant's previous studies in Type 2 diabetes patients, the procedures performed using system 10 were well-tolerated and proved to be safe in this study of patients with both NAFLD/NASH and Type 2 diabetes.

Results of an applicant-sponsored human clinical study using the systems, devices, and methods of the present inventive concepts are presented immediately herebelow. In the study, 24 patients received 5 ablations via a functional assembly 130 with a treatment length of 20 mm. Procedure time was reduced from 67 minutes to 45 minutes. Successful ablations resulted in 97% of those intended. Results at 3 months for the treated patients were as follows: mean HbA1c level was reduced by 1% (e.g. an HbA1c relative reduction of 12%) with a responder rate of 19/23 (83%); mean c-Peptide level was reduced by 0.5 ng/ml (16% reduction) with a responder rate of 17/24 (70%); mean HOMA-IR level was reduced by 1.9 (31.6% reduction) with a responder rate of 17/21 (81%); mean ALT level was reduced by 10.4 units/liter (29% reduction) with a responder rate of 18/23 (78%); mean PDFF level was reduced by 7% (37.6% reduction) with a responder rate of 16/17 (94%); 10 yr-ASCVD had a responder rate of 11/21 (52%); mean TG/HDL ratio was reduced by 1.53 (33.5% reduction) with a responder rate of 20/23 (87%); mean ferritin level was reduced by 22 ng/ml (24% reduction) with a responder rate of 23/23 (100%); and mean weight level was reduced by 3.1 kg (3% reduction) with a responder rate of 19/24 (80%).

Systems of the present inventive concepts (e.g. system 10 described herein) can be configured to treat Type 2 diabetes patients that are receiving insulin therapy (i.e. taking insulin), such that their insulin therapy can potentially be discontinued while the metabolic conditions of these patients is improved or at least maintained (e.g. HbA1c level or other metabolic condition marker is not made significantly worse by the removal of insulin therapy). For example, a duodenal mucosal treatment can be performed using catheter 100 and console 200, and an agent 420 can be delivered to the patient, such as an agent 420 comprising a GLP-1 receptor agonist. In some embodiments, the GLP-1 receptor agonist delivered can be liraglutide. Agent 420 can be delivered to the patient shortly after the time of the duodenal mucosal treatment (e.g. that day or the next day), or after a duration of time thereafter (e.g. 14 days after the mucosal treatment). In some embodiments, agent 420 comprises liraglutide that is delivered at a rate of at least 0.6 mg/day, or a rate of no more than 3.0 mg/day, such as a rate of approximately 1.8 mg/day (e.g. or equivalent dosages of a similar agent). The patient selected for treatment can have a C-peptide level above a threshold (e.g. indicating sufficient pancreatic function), and an HbA1c level below a threshold, such as when the treated patient has a C-peptide level of at least 0.5 nmol/l and an HbA1C level of no more than 64 mmol/mol. The insulin therapy of the patient (prior to the procedure) can comprise the taking of a daily insulin amount of not more than 1 Unit of insulin per kilogram of patient body weight (e.g. via one or more daily insulin injections). The duodenal mucosal treatment can comprise a heat ablation (e.g. via a hot fluid-filled balloon or direct ablation of steam and/or other hot fluid to tissue), cryogenic energy ablation, radiofrequency energy ablation, other energy-based ablation, and/or other treatment of the duodenal mucosa. A tissue expansion procedure can be performed as part of the treatment, such as a tissue expansion performed prior to energy delivery that creates a safety margin of tissue, as described herein (e.g. to avoid damage to tissue beyond the submucosal tissue of the patient's duodenum during energy delivery). The treatment can include delivery of an agent to mucosal tissue, such as an agent that denatures and/or otherwise alters the duodenal mucosa. The treatment can include treatment of one or more axial segments of duodenum, such as at least two, three, four, and/or five axial segments of duodenum. Each segment of duodenum treated can receive a full circumferential (360°) energy delivery, or a partial circumferential energy delivery, such as a delivery of ablation energy. A full circumferential energy delivery can correlate to the full circumferential portion of duodenal mucosa receiving energy being ablated, or a partial amount of mucosal ablation can occur, such as due to an ablation efficiency less than 100% (e.g. some of the mucosal tissue receiving energy does not achieve a time period at sufficient temperature to achieve necrosis of the tissue). In some embodiments, partial circumferential ablation (via energy delivery of 3600 or less) can be performed to avoid undesired circumferential ablation (e.g. circumferential ablation that may result in an undesired stricture being formed). At least 3 cm of duodenum can receive treatment, such as at least 6 cm or at least 9 cm of cumulative axial length to be treated (e.g. in one or more simultaneous and/or sequential treatments of two or more segments of duodenum). In some embodiments, an amount of duodenal mucosal tissue treated comprises a surface area (e.g. an exposed surface area comprising the inner surface of the duodenum) of at least 6.28 cm² (e.g. corresponding to a cumulative length of at least 2 cm of at least a 2 cm diameter duodenal lumen, and an ablation energy delivery efficiency of at least 50%, where at least 50% of the mucosal tissue corresponding to the surface area is ablated), such as a surface area of at least 9.42 cm², 18.84 cm², and/or at least 28.27 cm² (correlating to a similar minimum diameter of 2 cm, a similar ablation efficiency of 50%, but a minimum 3 cm, 6 cm, and/or 9 cm long segment, respectively). In some embodiments, at least 25% of the post-papillary duodenal mucosa is treated, such as when at least 50% of the post-papillary duodenal mucosa is treated. In the treatment procedures, treatment of the pre-papillary duodenum and papilla is avoided (e.g. sufficient energy to damage tissue in those areas is prevented).

As described hereabove, treatment of a Type 2 diabetes patient receiving insulin therapy via system 10 can allow the patient to stop taking insulin, while improving and/or at least maintaining one or more metabolic conditions after the taking of insulin has stopped. For example, HbA1c level can be maintained or reduced, such as to have an HbA1c level less than or equal to 59 mmol/mol. Alternatively or additionally. the patient can maintain and/or improve the level of one or more of: BMI, FPG, Insulin, HOMA-IR, ALT, MRI-PDFF, and/or peak glucose, such as is demonstrated below in reference to Table G.

Applicant has conducted studies in Type 2 diabetes patients that were each receiving insulin therapy as part of their treatment for Type 2 diabetes. Patients in the study received a duodenal mucosal treatment as well as the administration of a GLP-1 receptor agonist, using system 10 and consistent with the present inventive concepts. This therapeutic approach was shown to improve metabolic physiology of the patient while eliminating the insulin therapy. Each potential patient was screened, confirming an HbA1c level that was less than or equal to 64 mmol/mol, and a C-peptide level of at least 0.5 nmol/l. In the study, 16 patients were selected for treatment and these patients had the following characteristics: age 61±8 years; HbA1c 58.5±5.4 mmol/mol; and BMI 31±6.2 kg/m². Each treated patient was taking insulin, and at a level of no more than 1 Unit per kilogram of body weight per day.

Each patient received a duodenal mucosal ablation using catheter 100 and console 200, and their insulin therapy was discontinued. At 14 days after the ablation procedure, each patient started taking liraglutide (i.e. agent 420 comprising liraglutide), which was titrated to 1.8 mg/day. Liraglutide is a glucagon-like peptide-1 receptor agonist (GLP-1 receptor agonist) also known as incretin mimetics. Liraglutide increases insulin release from the pancreas and decreases excessive glucagon release.

A primary endpoint of the study was to determine what percentage of patients would have an HbA1c less than or equal to 59 mmol/mol while not taking insulin. As of the filing date of this application, 12 of the patients have reached six months, and 10 of these 12 patients (83%) have achieved the HbA1c goal. Table G herebelow shows metabolic information for these patients.

TABLE G

| | A: | B: Six | C: |
|---|---|---|---|
| | Baseline | month | |
| | Data | Data | p-value |
| BMI (kg/m²) | 31 ± 6.1 | 28.6 ± 5.4 | <0.001 |
| HbA1c (mmol/mol) | 58.5 ± 5.4 | 52.3 ± 6.2 | 0.002 |
| FPG (mmol/l) | 10.3 ± 2.1 | 7.8 ± 1.4 | 0.086 |
| Insulin (pmol/l) | 120 ± 60 | 64 ± 49 | 0.011 |
| HOMA-IR | 8.08 ± 4.4 | 3.4 ± 3 | 0.003 |
| ALT (U/l) | 25.9 ± 8.7 | 18 ± 4.8 | 0.008 |
| MRI-PDFF (%) | 8.5 ± 4.6 | 5.2 ± 3.2 | 0.064 |
| MRI-PDFF % | | −44.2 | |
| change vs. baseline | | | |
| Peak glucose | 16.0 ± 1.8 | 12.1 ± 1.7 | <0.001 |
| (mmol/l) | | | |

The baseline data represented by column A was collected from 12 patients that have reached a six month follow-up time period. The six month data represented by column B was collected from 10 of the 12 patients that were positive responders to the therapy provided by system 10 (e.g. remained off of insulin therapy and had an HbA1c level of no more than 59 mmol/mol).

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. (canceled)

2. A method of treating a medical condition of a patient, the method comprising:
selecting a patient diagnosed with type 2 diabetes that is being treated with daily insulin;
performing a tissue treatment procedure comprising treating one or more segments of the selected patient's intestinal tissue; and
subsequent to performing the tissue treatment procedure, administering an agent comprising a GLP-1 receptor agonist to the patient;
wherein the method provides a therapeutic benefit to the patient.

3. The method according to claim 2, wherein the method further comprises the patient stopping the taking of insulin.

4. The method according to claim 2, wherein the therapeutic benefit comprises a reduction in the patient's daily insulin requirements.

5. The method according to claim 2, wherein the therapeutic benefit comprises an elimination of daily insulin requirements.

6. The method according to claim 2, wherein the therapeutic benefit comprises a reduction in the patient's HbA1c levels.

7. The method according to claim 6, wherein the patient's HbA1c levels are reduced by at least 0.7% three months after the tissue treatment procedure.

8. The method according to claim 6, wherein the patient's HbA1c levels are reduced by approximately 2.18% three months after the tissue treatment procedure.

9. The method according to claim 2, wherein the therapeutic benefit comprises a reduction in the patient's hepatic fat content.

10. The method according to claim 9, wherein the patient's hepatic fat content is reduced by at least 10%.

11. The method according to claim 9, wherein the patient's hepatic fat content is reduced by approximately 36%.

12. The method according to claim 2, wherein the intestinal tissue treated comprises duodenal tissue.

13. The method according to claim 12, wherein each segment of duodenal tissue treated comprises an axial length of at least 3 cm.

14. The method according to claim 12, wherein the duodenal tissue treated comprises a cumulative length axial length of at least 3 cm.

15. The method according to claim 12, wherein the duodenal tissue treated comprises a cumulative length axial length of at least 6 cm.

16. The method according to claim 12, wherein the duodenal tissue treated comprises a cumulative length axial length of at least 8 cm.

17. The method according to claim 12, wherein the duodenal tissue treated comprises a cumulative length axial length of at least 9 cm.

18. The method according to claim 2, wherein the tissue treatment procedure comprises ablation.

19. The method according to claim 18, wherein the tissue treatment procedure comprises ablation via hot fluid.

20. The method according to claim 18, wherein the tissue treatment procedure comprises ablation via an energy type selected from the group consisting of: electromagnetic; radiofrequency; light; laser light; sound; ultrasound; chemical; cryogenic; and combinations thereof.

21. The method according to claim 2, wherein the tissue treatment procedure further comprises a tissue expansion procedure.

22. The method according to claim 21, wherein the tissue expansion procedure comprises expansion of submucosal tissue proximate the one or more segments of the selected patient's intestinal tissue.

23. The method according to claim 2, wherein the agent is administered to the patient 14 days after the tissue treatment procedure.

24. The method according to claim 2, wherein the therapeutic benefit is present at least six months after the tissue treatment procedure.

25. The method according to claim 2, wherein the selected patient has not achieved good glycemic control with daily insulin.

26. The method according to claim 25, wherein good glycemic control comprises an HbA1c level less than 7.5%, such as less than 7%.

27. The method according to claim 25, wherein good glycemic control comprises an HbA1c level less than 7%.
